(19) United States
(12) Patent Application Publication
(10) Pub. No.: US 2025/0009780 A1
(43) Pub. Date: Jan. 9, 2025

(54) AGENTS ENCODING CLDN6 AND CD3 BINDING ELEMENTS FOR TREATING CLDN6-POSITIVE CANCERS

(71) Applicant: BIONTECH SE, Mainz (DE)

(72) Inventors: Ugur SAHIN, Mainz (DE); Christiane STADLER, Mainz (DE); Claudia LINDEMANN, Mainz (DE); Ursula ELLINGHAUS, Mainz (DE); Hayat BÄHR-MAHMUD, Mainz (DE); Leyla FISCHER, Mainz (DE); Anuhar CHATURVEDI, Mainz (DE); Gábor BOROS, Mainz (DE); Jonas REINHOLZ, Mainz (DE); Sergey BESSONOV, Mainz (DE); Katalin KARIKÓ, Mainz (DE)

(21) Appl. No.: 18/578,844

(22) PCT Filed: Jul. 13, 2022
(86) PCT No.: PCT/EP2022/069659

§ 371 Date: Jan. 12, 2024

(30) Foreign Application Priority Data
Jul. 15, 2021 (WO) PCT/EP2021/069869

(51) Int. Cl. A61K 31/7115; A61K 9/00; A61K 9/127; A61K 9/51; A61K 45/06; A61P 35/00; C07K 16/28

(52) U.S. Cl. CPC A61K 31/7115; A61K 9/0019; A61K 9/127; A61K 9/5123; A61K 45/06; A61P 35/00; C07K 16/2809; C07K 2317/31; C07K 2317/522; C07K 2317/565

(57) ABSTRACT

The present invention generally relates to binding agents that are at least bispecific for the binding to CD3 and CLDN6, i.e., they are capable of binding to at least CD3 and CLDN6. Specifically, the present invention relates to RNA encoding these binding agents which may be used in the treatment or prevention of cancer in a subject.

### CROSS-REFERENCE TO RELATED APPLICATIONS

This application is a National Stage Entry of International Application Number PCT/EP2022/069659, which was filed on Jul. 13, 2022 and claimed priority to International Application Number PCT/EP2021/069869, which was filed on Jul. 15, 2021. The contents of each of the aforementioned applications are incorporated herein by reference in their entireties.

### SEQUENCE LISTING

The computer-readable Sequence Listing submitted on Jan. 12, 2024 and identified as follows: 56,161 bytes ST.26 XML document file named “026156-8065 Sequence Listing.xml,” created Jan. 11, 2024, is incorporated herein by reference in its entirety.

Cancer is the second leading cause of death globally and in 2020 was estimated to be responsible for 10 million deaths. In general, once a solid tumor has metastasized, with a few exceptions such as germ cell and some carcinoid tumors, 5-year survival rarely exceeds 25%.

Refinements in conventional therapies such as chemotherapy, radiotherapy, surgery, and targeted therapies and recent advances in immunotherapies have improved outcomes in patients with advanced solid tumors. In the last few years, the Food and Drug Administration (FDA) and European Medicines Agency (EMA) have approved several immune checkpoint inhibitors for the treatment of patients with multiple cancer types, mainly solid tumors. These approvals have dramatically changed the landscape of cancer treatment.

The poor prognosis in metastasized or locally advanced cancer highlights the need for additional treatment approaches. One such approach is that of targeted therapies, an ever-evolving field with promising modalities.

CLDN6 belongs to the PMP-22/EMP/MP20/Claudin superfamily of tetraspanin membrane proteins (Pfam database ID: PF00822) that are involved in formation of the apical tight-junction complex in epithelial and endothelial cellular sheets, with an important role in the maintenance of cell polarity (Krause G. et al., Biochim Biophys Acta. 2008; 1778(3):631-645). Importantly, the expression of claudin proteins is restricted to cellular tight junctions, and are accessible only for ion transport under standard physiological conditions (Krause G. et al., Biochim Biophys Acta. 2008; 1778(3):631-645). Otherwise, little else is known about the in vivo function of CLDN6. CLDN6 has four transmembrane helices with its N- and C-terminus extending into the cytoplasm. The short N-terminal sequence of CLDN6 is followed by a large extracellular loop (EL1), a short intracellular loop, a second extracellular loop (EL2), and the C-terminal cytoplasmic tail (Colegio O. R. et al., Am J Physiol Cell Physiol. 2002; 283(1):C142-C147).

No isoforms of CLDN6 have been identified so far (Lal-Nag M. et al., Genome Biol. 2009; 10(8):235). The claudin family members CLDN3, CLDN4 and CLDN9 share sequence homology with CLDN6. CLDN3 and CLDN4 are commonly expressed in normal epithelial cells of the lung, liver, breast, pancreas, kidney and gut (Kwon M. et al., Int J Mol Sci. 2013; 14(9):18148-18180). CLDN9 expression is absent from the vast majority of normal tissues; however, CLDN9 expression in cochlea and vestibule of the mouse inner ear has been reported (Kitajiri S. I. et al., Hear Res. 2004; 187(1-2):25-34; Nakano Y. et al., PLoS Genet. 2009; 5(8):e1000610), as has been a link between CLDN9 gene truncation and auditory impairment in humans (Sineni C. et al., Human genetics 2019; 138(10):1071-1075).

The oncofetal protein CLDN6 is expressed almost exclusively in embryonic stem cells, is then rapidly downregulated during differentiation into the neural or cardiac lineages and is not expressed in normal adult tissues other than placenta (Assou S. et al., Stem Cells. 2007; 25(4):961-973; Ben-David U. et al., Nat Commun. 2013; 4:1992; Reinhard K. et al., Science. 2020; 367(6476):446-453). CLDN6 is expressed in various human cancer types including testicular, ovarian, endometrial and lung cancer. A representative study showed that about 93% of testicular cancer of all histological subtypes stained highly positive for CLDN6 defined by a staining intensity ≥2+. Moreover, 56% of ovarian cancer stained positive for CLDN6, of which 20 to 25% displayed high (≥2+) cell membrane staining in over 50% of tumor cells. Compared to primary ovarian cancer, the frequency of CLDN6-positive samples was significantly increased in metastasis lesions (72%; data not shown), associating CLDN6 expression with disease progression. 23% of endometrial and 11% of lung carcinomas stained positive for CLDN6 of which 10 to 15% and 2 to 5% displayed staining intensities ≥2+, respectively.

It has been an object of the invention to provide novel agents and methods for the therapy of CLDN6-positive cancer diseases.

In some embodiments, the solution of the problem underlying the invention is based on the concept of administering RNA that is expressed by cells of a patient to express polypeptide chains forming a binding agent that comprises two binding domains that are specific for CLDN6 expressed by cancer cells and a binding domain that is specific for CD3 expressed by T cells, thus making it possible to target the cytotoxic effect of the T cells to the cancer cells.

### SUMMARY

The present invention generally relates to binding agents that are at least bispecific for the binding to CD3 and CLDN6, i.e., they are capable of binding to at least CD3 and CLDN6. Specifically, the present invention relates to RNA encoding these binding agents which may be used in the treatment or prevention of cancer in a subject. In particular, RNA encoding a binding agent disclosed herein may be administered to provide (following expression of the RNA by appropriate target cells) binding agent for targeting CD3 and CLDN6.

Thus, a pharmaceutical composition described herein may comprise as the active principle single-stranded RNA that may be translated into the respective encoded polypeptide upon entering cells of a recipient. In addition to sequences encoding the binding agent, the RNA may contain one or more structural elements optimized for maximal efficacy of the RNA with respect to stability and translational efficiency (5′-cap, 5′-UTR, 3′-UTR, poly(A)-tail). In some embodiments, the RNA contains all of these elements.

The RNA described herein may be complexed with proteins and/or lipids, preferably lipids, to generate RNA particles for administration. Different RNAs may be complexed together or complexed separately with proteins and/or lipids to generate RNA-particles for administration.

In one aspect, the present invention provides a composition or medical preparation comprising:
(i) a first RNA encoding a first polypeptide chain comprising a variable region of a heavy chain (VH) derived from an immunoglobulin with specificity for CD3 (VH(CD3)), a variable region of a heavy chain (VH) derived from an immunoglobulin with specificity for CLDN6 (VH(CLDN6)) and a variable region of a light chain (VL) derived from an immunoglobulin with specificity for CLDN6 (VL(CLDN6)); and
(ii) a second RNA encoding a second polypeptide chain comprising a variable region of a light chain (VL) derived from an immunoglobulin with specificity for CD3 (VL(CD3)), a variable region of a heavy chain (VH) derived from an immunoglobulin with specificity for CLDN6 (VH(CLDN6)) and a variable region of a light chain (VL) derived from an immunoglobulin with specificity for CLDN6 (VL(CLDN6)).

In some embodiments, the first polypeptide chain interacts with the second polypeptide chain to form a binding domain with specificity for CD3 and two binding domains with specificity for CLDN6.

In some embodiments:
the VH(CD3) of the first polypeptide chain and the VL(CD3) of the second polypeptide chain interact to form a binding domain with specificity for CD3,
the VH(CLDN6) and the VL(CLDN6) of the first polypeptide chain interact to form a binding domain with specificity for CLDN6, and
the VH(CLDN6) and the VL(CLDN6) of the second polypeptide chain interact to form a binding domain with specificity for CLDN6.

In one aspect, the present invention provides a composition or medical preparation comprising:
(i) a first RNA encoding a first polypeptide chain comprising a variable region VH(CD3), a variable region VH(CLDN6) and a variable region VL(CLDN6); and
(ii) a second RNA encoding a second polypeptide chain comprising a variable region VL(CD3), a variable region VH(CLDN6) and a variable region VL(CLDN6),
wherein the VH(CD3) of the first polypeptide chain and the VL(CD3) of the second polypeptide chain interact to form a binding domain with specificity for CD3,
wherein the VH(CLDN6) and the VL(CLDN6) of the first polypeptide chain interact to form a binding domain with specificity for CLDN6, and
wherein the VH(CLDN6) and the VL(CLDN6) of the second polypeptide chain interact to form a binding domain with specificity for CLDN6.

In some embodiments, the first and the second polypeptide chains comprise a constant region 1 of a heavy chain (CH1) derived from an immunoglobulin or a functional variant thereof and a constant region of a light chain (CL) derived from an immunoglobulin or a functional variant thereof.

In some embodiments, the immunoglobulin is IgG1.

In some embodiments, the IgG1 is human IgG1.

In some embodiments, the VH, the VL, and the CH1 on the first polypeptide chain are arranged, from N-terminus to C-terminus, in the order
VH(CD3)-CH1-VH(CLDN6)-VL(CLDN6), or
VH(CD3)-CH1-VL(CLDN6)-VH(CLDN6).

In some embodiments, the CH1 is connected to the VH(CLDN6) or VL(CLDN6) by a peptide linker. In one embodiment, the peptide linker comprises the amino acid sequence SGPGGGRS(G₄S)₂ or a functional variant thereof.

In some embodiments, the VH, the VL, and the CL on the second polypeptide chain are arranged, from N-terminus to C-terminus, in the order
VL(CD3)-CL-VH(CLDN6)-VL(CLDN6), or
VL(CD3)-CL-VL(CLDN6)-VH(CLDN6).

In some embodiments, the CL is connected to the VH(CLDN6) or VL(CLDN6) by a peptide linker. In some embodiments, the peptide linker comprises the amino acid sequence DVPGGS or a functional variant thereof.

In some embodiments, the VH(CLDN6) and the VL(CLDN6) are connected to one another by a peptide linker. In some embodiments, the peptide linker comprises the amino acid sequence (G₄S)ₓ or a functional variant thereof, wherein x is 2, 3, 4, 5 or 6. In one embodiment, the peptide linker comprises the amino acid sequence (G₄S)₄ or a functional variant thereof.

In some embodiments, the CH1 on the first polypeptide chain interacts with the CL on the second polypeptide chain.

In some embodiments, the VH(CD3) comprises CDR1, CDR2 and CDR3 of the amino acid sequence of amino acids 27 to 145 of SEQ ID NO: 4.

In some embodiments, the VH(CD3) comprises a CDR1 comprising the amino acid sequence GYTFTRYT or a functional variant thereof, a CDR2 comprising the amino acid sequence INPSRGYT or a functional variant thereof, and a CDR3 comprising the amino acid sequence ARYYDDHYSLDY or a functional variant thereof.

In some embodiments, the VH(CD3) comprises a CDR1 comprising the amino acid sequence GYTFTRYT or a functional variant thereof, a CDR2 comprising the amino acid sequence INPSRGYT or a functional variant thereof, and a CDR3 comprising the amino acid sequence ARYYDDHYCLDY or a functional variant thereof.

In some embodiments, the VH(CD3) comprises the amino acid sequence of amino acids 27 to 145 of SEQ ID NO: 4 or a functional variant thereof.

In some embodiments, the VL(CD3) comprises CDR1, CDR2 and CDR3 of the amino acid sequence of amino acids 27 to 132 of SEQ ID NO: 6.

In some embodiments, the VL(CD3) comprises a CDR1 comprising the amino acid sequence SSVSY or a functional variant thereof, a CDR2 comprising the amino acid sequence DTS or a functional variant thereof, and a CDR3 comprising the amino acid sequence QQWSSNPLT or a functional variant thereof.

In some embodiments, the VL(CD3) comprises the amino acid sequence of amino acids 27 to 132 of SEQ ID NO: 6 or a functional variant thereof.

In some embodiments, the VH(CLDN6) comprises CDR1, CDR2 and CDR3 of the amino acid sequence of amino acids 267 to 383 of SEQ ID NO: 4.

In some embodiments, the VH(CLDN6) comprises a CDR1 comprising the amino acid sequence GYSFTGYT or a functional variant thereof, a CDR2 comprising the amino acid sequence INPYNGGT or a functional variant thereof, and a CDR3 comprising the amino acid sequence ARDYGFVLDY or a functional variant thereof.

In some embodiments, the VH(CLDN6) comprises the amino acid sequence of amino acids 267 to 383 of SEQ ID NO: 4 or a functional variant thereof.

In some embodiments, the VL(CLDN6) comprises CDR1, CDR2 and CDR3 of the amino acid sequence of amino acids 404 to 510 of SEQ ID NO: 4.

In some embodiments, the VL(CLDN6) comprises CDR1, CDR2 and CDR3 of the amino acid sequence of amino acids 404 to 510 of SEQ ID NO: 4 and a serine residue in position +15 relative to CDR1 (corresponds to position 449 of SEQ ID NO: 4).

In some embodiments, the VL(CLDN6) comprises CDR1, CDR2 and CDR3 of the amino acid sequence of amino acids 404 to 510 of SEQ ID NO: 4 and a serine residue in position −3 relative to CDR2 (corresponds to position 449 of SEQ ID NO: 4).

In some embodiments, the VL(CLDN6) comprises CDR1, CDR2 and CDR3 of the amino acid sequence of amino acids 404 to 510 of SEQ ID NO: 4, a sequence having at least 99%, 98%, 97%, 96%, 95%, 90%, 85%, or 80% identity to the amino acid sequence of amino acids 404 to 510 of SEQ ID NO: 4, and a serine residue in the position corresponding to position 449 of SEQ ID NO: 4. In some embodiments, the VL(CLDN6) comprises a CDR1 comprising the amino acid sequence SSVSY or a functional variant thereof, a CDR2 comprising the amino acid sequence STS or a functional variant thereof, and a CDR3 comprising the amino acid sequence QQRSNYPPWT or a functional variant thereof.

In some embodiments, the VL(CLDN6) comprises the amino acid sequence of amino acids 404 to 510 of SEQ ID NO: 4 or a functional variant thereof.

In some embodiments, the VH(CD3) comprises CDR1, CDR2 and CDR3 of the amino acid sequence of amino acids 27 to 145 of SEQ ID NO: 4, the VL(CD3) comprises CDR1, CDR2 and CDR3 of the amino acid sequence of amino acids 27 to 132 of SEQ ID NO: 6, the VH(CLDN6) comprises CDR1, CDR2 and CDR3 of the amino acid sequence of amino acids 267 to 383 of SEQ ID NO: 4, and the VL(CLDN6) comprises CDR1, CDR2 and CDR3 of the amino acid sequence of amino acids 404 to 510 of SEQ ID NO: 4, and preferably a serine residue in position +15 relative to CDR1 (corresponds to position 449 of SEQ ID NO: 4) and/or a serine residue in position −3 relative to CDR2 (corresponds to position 449 of SEQ ID NO: 4).

In some embodiments:
the VH(CD3) comprises the amino acid sequence of amino acids 27 to 145 of SEQ ID NO: 4 or a functional variant thereof,
the VL(CD3) comprises the amino acid sequence of amino acids 27 to 132 of SEQ ID NO: 6 or a functional variant thereof,
the VH(CLDN6) comprises the amino acid sequence of amino acids 267 to 383 of SEQ ID NO: 4 or a functional variant thereof, and/or
the VL(CLDN6) comprises the amino acid sequence of amino acids 404 to 510 of SEQ ID NO: 4 or a functional variant thereof.

In some embodiments, the first polypeptide chain comprises the amino acid sequence of SEQ ID NO: 4 or a functional variant thereof.

In some embodiments, the second polypeptide chain comprises the amino acid sequence of SEQ ID NO: 6 or a functional variant thereof.

In some embodiments, the first polypeptide chain comprises the amino acid sequence of SEQ ID NO: 4 or a functional variant thereof and the second polypeptide chain comprises the amino acid sequence of SEQ ID NO: 6 or a functional variant thereof.

In some embodiments, at least one of the first polypeptide and the second polypeptide is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In some embodiments, each of the first polypeptide and the second polypeptide is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In some embodiments, the RNA comprises a modified nucleoside in place of uridine. In such a case there is preferably a modified nucleoside in place of each or essentially each uridine in the RNA.

In some embodiments, the modified nucleoside is selected from pseudouridine (ψ), N1-methyl-pseudouridine (m1ψ), and 5-methyl-uridine (m5U).

In some embodiments, at least one RNA comprises the 5′ cap m₂^{7,3′-O}Gppp(m₁^{2′-O})ApG.

In some embodiments, each RNA comprises the 5′ cap m₂^{7,3′-O}Gppp(m₁^{2′-O})ApG.

In some embodiments, at least one RNA comprises a 5′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.

In some embodiments, each RNA comprises a 5′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.

In some embodiments, at least one RNA comprises a 3′ UTR comprising the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9.

In some embodiments, each RNA comprises a 3′ UTR comprising the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9.

In some embodiments, at least one RNA comprises a poly-A sequence.

In some embodiments, each RNA comprises a poly-A sequence.

In some embodiments, the poly-A sequence comprises at least 100 nucleotides.

In some embodiments, the poly-A sequence comprises or consists of the nucleotide sequence of SEQ ID NO: 10.

In some embodiments:
(i) the first RNA and the second RNA are in a (w/w) ratio of about 1.75:1 to about 1.25:1, or about 1.5:1 to about 1.25:1, or preferably about 1.5:1; and/or
(ii) the first RNA and the second RNA comprise a modified nucleoside in place of each uridine; and/or
(iii) the first RNA and the second RNA comprise a modified nucleoside in place of each uridine, wherein the modified nucleoside is independently selected from pseudouridine (ψ), N1-methyl-pseudouridine (m1ψ), and 5-methyl-uridine (m5U); and/or
(iv) the first RNA and the second RNA comprise the 5′ cap m₂^{7,3′-O}Gppp(m₁^{2′-O})ApG; and/or (v) the first RNA and the second RNA comprise a 5′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8; and/or
(vi) the first RNA and the second RNA comprise a 3′ UTR comprising the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9; and/or
(vii) the first RNA and the second RNA comprise a poly-A tail comprising the nucleotide sequence of SEQ ID NO: 10.

In some embodiments:
(i) the first RNA and the second RNA are in a (w/w) ratio of about 1.75:1 to about 1.25:1, or about 1.5:1 to about 1.25:1, or preferably about 1.5:1;
(ii) the first RNA and the second RNA comprise a modified nucleoside in place of each uridine, wherein the modified nucleoside is N1-methyl-pseudouridine (m1ψ);
(iii) the first RNA and the second RNA comprise the 5′ cap m₂^{7,3′-O}Gppp(m₁^{2′-O})ApG; (iv) the first RNA and the second RNA comprise a 5′ UTR comprising the nucleotide sequence of SEQ ID NO: 8;
(v) the first RNA and the second RNA comprise a 3′ UTR comprising the nucleotide sequence of SEQ ID NO: 9; and
(vi) the first RNA and the second RNA comprise a poly-A tail comprising the nucleotide sequence of SEQ ID NO: 10.

In some embodiments:
(i) the first polypeptide chain comprises the amino acid sequence of SEQ ID NO: 4, or an amino acid sequence having at least 99%, 98%, 97%, 96%, 95%, 90%, 85%, or 80% identity to the amino acid sequence of SEQ ID NO: 4; and/or
(ii) the first RNA comprises the nucleotide sequence of SEQ ID NO: 5, or a nucleotide sequence having at least 99%, 98%, 97%, 96%, 95%, 90%, 85%, or 80% identity to the nucleotide sequence of SEQ ID NO: 5.

In some embodiments:
(i) the second polypeptide chain comprises the amino acid sequence of SEQ ID NO: 6, or an amino acid sequence having at least 99%, 98%, 97%, 96%, 95%, 90%, 85%, or 80% identity to the amino acid sequence of SEQ ID NO: 6; and/or
(ii) the second RNA comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7.

In one aspect, the present invention provides a composition or medical preparation comprising:
(i) a first RNA encoding a first polypeptide chain comprising the amino acid sequence of SEQ ID NO: 4, or an amino acid sequence having at least 99%, 98%, 97%, 96%, 95%, 90%, 85%, or 80% identity to the amino acid sequence of SEQ ID NO: 4; and
(ii) a second RNA encoding a second polypeptide chain comprising the amino acid sequence of SEQ ID NO: 6, or an amino acid sequence having at least 99%, 98%, 97%, 96%, 95%, 90%, 85%, or 80% identity to the amino acid sequence of SEQ ID NO: 6.

In some embodiments of all aspects, the first RNA comprises the nucleotide sequence of SEQ ID NO: 5, or a nucleotide sequence having at least 99%, 98%, 97%, 96%, 95%, 90%, 85%, or 80% identity to the nucleotide sequence of SEQ ID NO: 5.

In some embodiments of all aspects, the second RNA comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7.

In one aspect, the present invention provides a composition or medical preparation comprising: a first RNA comprising the nucleotide sequence of SEQ ID NO: 35 or a nucleotide sequence having at least 99%, 98%, 97%, 96%, 95%, 90%, 85%, or 80% identity to the nucleotide sequence of SEQ ID NO: 35 and/or a second RNA comprising the nucleotide sequence of SEQ ID NO: 36 or a nucleotide sequence having at least 99%, 98%, 97%, 96%, 95%, 90%, 85%, or 80% identity to the nucleotide sequence of SEQ ID NO: 36.

In one aspect, the present invention provides a composition or medical preparation comprising: a first RNA comprising the nucleotide sequence of SEQ ID NO: 5 and a second RNA comprising the nucleotide sequence of SEQ ID NO: 7, wherein the first RNA and the second RNA are present at a (w/w) ratio of first RNA to second RNA of about 1.5:1.

In some embodiments of all aspects, the RNA is mRNA.

In some embodiments of all aspects, the RNA is formulated as a liquid, formulated as a solid, or a combination thereof.

In some embodiments of all aspects, the RNA is formulated or is to be formulated for injection.

In some embodiments of all aspects, the RNA is formulated or is to be formulated for intravenous administration.

In some embodiments of all aspects, the RNA is formulated or is to be formulated as particles.

In some embodiments, the particles are lipid nanoparticles (LNP).

In some embodiments, the LNP particles comprise ((3-hydroxypropyl)azanediyl)bis(nonane-9,1-diyl)bis(2-butyloctanoate), 2-[(polyethylene glycol)-2000]-N,N-ditetradecylacetamide, 1,2-Distearoyl-sn-glycero-3-phosphocholine, and cholesterol.

In some embodiments of all aspects, the composition or medical preparation is a pharmaceutical composition.

In some embodiments, the pharmaceutical composition further comprises one or more pharmaceutically acceptable carriers, diluents and/or excipients.

In some embodiments of all aspects, the composition or medical preparation is a kit.

In some embodiments, the RNA and optionally the particle forming components are in separate vials.

In some embodiments, the composition or medical preparation further comprises instructions for use of the composition or medical preparation for treating or preventing cancer.

In one aspect, the present invention provides a composition or medical preparation described herein for pharmaceutical use.

In some embodiments, the pharmaceutical use comprises a therapeutic or prophylactic treatment of a disease or disorder.

In some embodiments, the therapeutic or prophylactic treatment of a disease or disorder comprises treating or preventing cancer.

In some embodiments, the therapeutic or prophylactic treatment of a disease or disorder further comprises administering a further therapy.

In some embodiments, the further therapy comprises one or more selected from the group consisting of: (i) surgery to excise, resect, or debulk a tumor, (ii) radiotherapy, and (iii) chemotherapy.

In some embodiments, the further therapy comprises administering a further therapeutic agent.

In some embodiments, the further therapeutic agent comprises an anti-cancer therapeutic agent.

In some embodiments, the composition or medical preparation described herein is for administration to a human.

In one aspect, the present invention provides a method of treating cancer in a subject comprising administering to the subject:
(i) a first RNA encoding a first polypeptide chain comprising a variable region of a heavy chain (VH) derived from an immunoglobulin with specificity for CD3 (VH(CD3)), a variable region of a heavy chain (VH) derived from an immunoglobulin with specificity for CLDN6 (VH(CLDN6)) and a variable region of a light chain (VL) derived from an immunoglobulin with specificity for CLDN6 (VL(CLDN6)); and
(ii) a second RNA encoding a second polypeptide chain comprising a variable region of a light chain (VL) derived from an immunoglobulin with specificity for CD3 (VL(CD3)), a variable region of a heavy chain (VH) derived from an immunoglobulin with specificity for CLDN6 (VH(CLDN6)) and a variable region of a light chain (VL) derived from an immunoglobulin with specificity for CLDN6 (VL(CLDN6)).

In some embodiments, the first polypeptide chain interacts with the second polypeptide chain to form a binding domain with specificity for CD3 and two binding domains with specificity for CLDN6.

In some embodiments:
the VH(CD3) of the first polypeptide chain and the VL(CD3) of the second polypeptide chain interact to form a binding domain with specificity for CD3,
the VH(CLDN6) and the VL(CLDN6) of the first polypeptide chain interact to form a binding domain with specificity for CLDN6, and
the VH(CLDN6) and the VL(CLDN6) of the second polypeptide chain interact to form a binding domain with specificity for CLDN6.

In one aspect, the present invention provides a method of treating cancer in a subject comprising administering to the subject:
(i) a first RNA encoding a first polypeptide chain comprising a variable region VH(CD3), a variable region VH(CLDN6) and a variable region VL(CLDN6); and
(ii) a second RNA encoding a second polypeptide chain comprising a variable region VL(CD3), a variable region VH(CLDN6) and a variable region VL(CLDN6),
wherein the VH(CD3) of the first polypeptide chain and the VL(CD3) of the second polypeptide chain interact to form a binding domain with specificity for CD3,
wherein the VH(CLDN6) and the VL(CLDN6) of the first polypeptide chain interact to form a binding domain with specificity for CLDN6, and
wherein the VH(CLDN6) and the VL(CLDN6) of the second polypeptide chain interact to form a binding domain with specificity for CLDN6.

In some embodiments, the first and the second polypeptide chains comprise a constant region 1 of a heavy chain (CH1) derived from an immunoglobulin or a functional variant thereof and a constant region of a light chain (CL) derived from an immunoglobulin or a functional variant thereof.

In some embodiments, the immunoglobulin is IgG1.

In some embodiments, the IgG1 is human IgG1.

In some embodiments, the VH, the VL, and the CH1 on the first polypeptide chain are arranged, from N-terminus to C-terminus, in the order
VH(CD3)-CH1-VH(CLDN6)-VL(CLDN6), or
VH(CD3)-CH1-VL(CLDN6)-VH(CLDN6).

In some embodiments, the CH1 is connected to the VH(CLDN6) or VL(CLDN6) by a peptide linker. In some embodiments, the peptide linker comprises the amino acid sequence SGPGGGRS(G₄S)₂ or a functional variant thereof.

In some embodiments, the VH, the VL, and the CL on the second polypeptide chain are arranged, from N-terminus to C-terminus, in the order
VL(CD3)-CL-VH(CLDN6)-VL(CLDN6), or
VL(CD3)-CL-VL(CLDN6)-VH(CLDN6).

In some embodiments, the CL is connected to the VH(CLDN6) or VL(CLDN6) by a peptide linker. In some embodiments, the peptide linker comprises the amino acid sequence DVPGGS or a functional variant thereof.

In some embodiments, the VH(CLDN6) and the VL(CLDN6) are connected to one another by a peptide linker. In some embodiments, the peptide linker comprises the amino acid sequence (G₄S)ₓ or a functional variant thereof, wherein x is 2, 3, 4, 5 or 6. In some embodiments, the peptide linker comprises the amino acid sequence (G₄S)₄ or a functional variant thereof.

In some embodiments, the CH1 on the first polypeptide chain interacts with the CL on the second polypeptide chain.

In some embodiments, the VH(CD3) comprises CDR1, CDR2 and CDR3 of the amino acid sequence of amino acids 27 to 145 of SEQ ID NO: 4.

In some embodiments, the VH(CD3) comprises a CDR1 comprising the amino acid sequence GYTFTRYT or a functional variant thereof, a CDR2 comprising the amino acid sequence INPSRGYT or a functional variant thereof, and a CDR3 comprising the amino acid sequence ARYYDDHYSLDY or a functional variant thereof.

In some embodiments, the VH(CD3) comprises a CDR1 comprising the amino acid sequence GYTFTRYT or a functional variant thereof, a CDR2 comprising the amino acid sequence INPSRGYT or a functional variant thereof, and a CDR3 comprising the amino acid sequence ARYYDDHYCLDY or a functional variant thereof.

In some embodiments, the VH(CD3) comprises the amino acid sequence of amino acids 27 to 145 of SEQ ID NO: 4 or a functional variant thereof.

In some embodiments, the VL(CD3) comprises CDR1, CDR2 and CDR3 of the amino acid sequence of amino acids 27 to 132 of SEQ ID NO: 6.

In some embodiments, the VL(CD3) comprises a CDR1 comprising the amino acid sequence SSVSY or a functional variant thereof, a CDR2 comprising the amino acid sequence DTS or a functional variant thereof, and a CDR3 comprising the amino acid sequence QQWSSNPLT or a functional variant thereof.

In some embodiments, the VL(CD3) comprises the amino acid sequence of amino acids 27 to 132 of SEQ ID NO: 6 or a functional variant thereof.

In some embodiments, the VH(CLDN6) comprises CDR1, CDR2 and CDR3 of the amino acid sequence of amino acids 267 to 383 of SEQ ID NO: 4.

In some embodiments, the VH(CLDN6) comprises a CDR1 comprising the amino acid sequence GYSFTGYT or a functional variant thereof, a CDR2 comprising the amino acid sequence INPYNGGT or a functional variant thereof, and a CDR3 comprising the amino acid sequence ARDYGFVLDY or a functional variant thereof.

In some embodiments, the VH(CLDN6) comprises the amino acid sequence of amino acids 267 to 383 of SEQ ID NO: 4 or a functional variant thereof.

In some embodiments, the VL(CLDN6) comprises CDR1, CDR2 and CDR3 of the amino acid sequence of amino acids 404 to 510 of SEQ ID NO: 4.

In some embodiments, the VL(CLDN6) comprises CDR1, CDR2 and CDR3 of the amino acid sequence of amino acids 404 to 510 of SEQ ID NO: 4 and a serine residue in position +15 relative to CDR1 (corresponds to position 449 of SEQ ID NO: 4).

In some embodiments, the VL(CLDN6) comprises CDR1, CDR2 and CDR3 of the amino acid sequence of amino acids 404 to 510 of SEQ ID NO: 4 and a serine residue in position −3 relative to CDR2 (corresponds to position 449 of SEQ ID NO: 4).

In some embodiments, the VL(CLDN6) comprises CDR1, CDR2 and CDR3 of the amino acid sequence of amino acids 404 to 510 of SEQ ID NO: 4, a sequence having at least 99%, 98%, 97%, 96%, 95%, 90%, 85%, or 80% identity to the amino acid sequence of amino acids 404 to 510 of SEQ ID NO: 4, and a serine residue in the position corresponding to position 449 of SEQ ID NO: 4.

In some embodiments, the VL(CLDN6) comprises a CDR1 comprising the amino acid sequence SSVSY or a functional variant thereof, a CDR2 comprising the amino acid sequence STS or a functional variant thereof, and a CDR3 comprising the amino acid sequence QQRSNYPPWT or a functional variant thereof.

In some embodiments, the VL(CLDN6) comprises the amino acid sequence of amino acids 404 to 510 of SEQ ID NO: 4 or a functional variant thereof.

In some embodiments, the VH(CD3) comprises CDR1, CDR2 and CDR3 of the amino acid sequence of amino acids 27 to 145 of SEQ ID NO: 4, the VL(CD3) comprises CDR1, CDR2 and CDR3 of the amino acid sequence of amino acids 27 to 132 of SEQ ID NO: 6, the VH(CLDN6) comprises CDR1, CDR2 and CDR3 of the amino acid sequence of amino acids 267 to 383 of SEQ ID NO: 4, and the VL(CLDN6) comprises CDR1, CDR2 and CDR3 of the amino acid sequence of amino acids 404 to 510 of SEQ ID NO: 4 and preferably a serine residue in position +15 relative to CDR1 (corresponds to position 449 of SEQ ID NO: 4) and/or a serine residue in position −3 relative to CDR2 (corresponds to position 449 of SEQ ID NO: 4).

In some embodiments:
the VH(CD3) comprises the amino acid sequence of amino acids 27 to 145 of SEQ ID NO: 4 or a functional variant thereof,
the VL(CD3) comprises the amino acid sequence of amino acids 27 to 132 of SEQ ID NO: 6 or a functional variant thereof,
the VH(CLDN6) comprises the amino acid sequence of amino acids 267 to 383 of SEQ ID NO: 4 or a functional variant thereof, and/or
the VL(CLDN6) comprises the amino acid sequence of amino acids 404 to 510 of SEQ ID NO: 4 or a functional variant thereof.

In some embodiments, the first polypeptide chain comprises the amino acid sequence of SEQ ID NO: 4 or a functional variant thereof.

In some embodiments, the second polypeptide chain comprises the amino acid sequence of SEQ ID NO: 6 or a functional variant thereof.

In some embodiments, the first polypeptide chain comprises the amino acid sequence of SEQ ID NO: 4 or a functional variant thereof and the second polypeptide chain comprises the amino acid sequence of SEQ ID NO: 6 or a functional variant thereof.

In some embodiments, at least one of the first polypeptide and the second polypeptide is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In some embodiments, each of the first polypeptide and the second polypeptide is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In some embodiments, the RNA comprises a modified nucleoside in place of uridine. In such a case there is preferably a modified nucleoside in place of each or essentially each uridine in the RNA.

In some embodiments, the modified nucleoside is selected from pseudouridine (ψ), N1-methyl-pseudouridine (m1ψ), and 5-methyl-uridine (m5U).

In some embodiments, at least one RNA comprises the 5′ cap m₂^{7,3′-O}Gppp(m₁^{2′-O})ApG.

In some embodiments, each RNA comprises the 5′ cap m₂^{7,3′-O}Gppp(m₁^{2′-O})ApG.

In some embodiments, at least one RNA comprises a 5′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.

In some embodiments, each RNA comprises a 5′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.

In some embodiments, at least one RNA comprises a 3′ UTR comprising the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9.

In some embodiments, each RNA comprises a 3′ UTR comprising the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9.

In some embodiments, at least one RNA comprises a poly-A sequence.

In some embodiments, each RNA comprises a poly-A sequence.

In some embodiments, the poly-A sequence comprises at least 100 nucleotides.

In some embodiments, the poly-A sequence comprises or consists of the nucleotide sequence of SEQ ID NO: 10.

In some embodiments:
(i) the first RNA and the second RNA are in a (w/w) ratio of about 1.75:1 to about 1.25:1, or about 1.5:1 to about 1.25:1, or preferably about 1.5:1; and/or
(ii) the first RNA and the second RNA comprise a modified nucleoside in place of each uridine; and/or
(iii) the first RNA and the second RNA comprise a modified nucleoside in place of each uridine, wherein the modified nucleoside is independently selected from pseudouridine (ψ), N1-methyl-pseudouridine (m1ψ), and 5-methyl-uridine (m5U); and/or
(iv) the first RNA and the second RNA comprise the 5′ cap m₂^{7,3-O}Gppp(m₁^{2′-O})ApG; and/or
(v) the first RNA and the second RNA comprise a 5′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8; and/or
(vi) the first RNA and the second RNA comprise a 3′ UTR comprising the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9; and/or
(vii) the first RNA and the second RNA comprise a poly-A tail comprising the nucleotide sequence of SEQ ID NO: 10.

In some embodiments:
(i) the first RNA and the second RNA are in a (w/w) ratio of about 1.75:1 to about 1.25:1, or about 1.5:1 to about 1.25:1, or preferably about 1.5:1;
(ii) the first RNA and the second RNA comprise a modified nucleoside in place of each uridine, wherein the modified nucleoside is N1-methyl-pseudouridine (m1ψ);
(iii) the first RNA and the second RNA comprise the 5′ cap m₂^{7,3′-O}Gppp(m₁^{2′-O})ApG;
(iv) the first RNA and the second RNA comprise a 5′ UTR comprising the nucleotide sequence of SEQ ID NO: 8;
(v) the first RNA and the second RNA comprise a 3′ UTR comprising the nucleotide sequence of SEQ ID NO: 9; and
(vi) the first RNA and the second RNA comprise a poly-A tail comprising the nucleotide sequence of SEQ ID NO: 10.

In some embodiments:
(i) the first polypeptide chain comprises the amino acid sequence of SEQ ID NO: 4, or an amino acid sequence having at least 99%, 98%, 97%, 96%, 95%, 90%, 85%, or 80% identity to the amino acid sequence of SEQ ID NO: 4; and/or
(ii) the first RNA comprises the nucleotide sequence of SEQ ID NO: 5, or a nucleotide sequence having at least 99%, 98%, 97%, 96%, 95%, 90%, 85%, or 80% identity to the nucleotide sequence of SEQ ID NO: 5.

In some embodiments:
(i) the second polypeptide chain comprises the amino acid sequence of SEQ ID NO: 6, or an amino acid sequence having at least 99%, 98%, 97%, 96%, 95%, 90%, 85%, or 80% identity to the amino acid sequence of SEQ ID NO: 6; and/or
(ii) the second RNA comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7.

In one aspect, the present invention provides a method of treating cancer in a subject comprising administering to the subject:
(i) a first RNA encoding a first polypeptide chain comprising the amino acid sequence of SEQ ID NO: 4, or an amino acid sequence having at least 99%, 98%, 97%, 96%, 95%, 90%, 85%, or 80% identity to the amino acid sequence of SEQ ID NO: 4; and
(ii) a second RNA encoding a second polypeptide chain comprising the amino acid sequence of SEQ ID NO: 6, or an amino acid sequence having at least 99%, 98%, 97%, 96%, 95%, 90%, 85%, or 80% identity to the amino acid sequence of SEQ ID NO: 6.

In some embodiments of all aspects, the first RNA comprises the nucleotide sequence of SEQ ID NO: 5, or a nucleotide sequence having at least 99%, 98%, 97%, 96%, 95%, 90%, 85%, or 80% identity to the nucleotide sequence of SEQ ID NO: 5.

In some embodiments of all aspects, the second RNA comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7.

In one aspect, the present invention provides a method of treating cancer in a subject comprising administering to the subject: a first RNA comprising the nucleotide sequence of SEQ ID NO: 35 or a nucleotide sequence having at least 99%, 98%, 97%, 96%, 95%, 90%, 85%, or 80% identity to the nucleotide sequence of SEQ ID NO: 35 and/or a second RNA comprising the nucleotide sequence of SEQ ID NO: 36 or a nucleotide sequence having at least 99%, 98%, 97%, 96%, 95%, 90%, 85%, or 80% identity to the nucleotide sequence of SEQ ID NO: 36.

In one aspect, the present invention provides a method of treating cancer in a subject comprising administering to the subject: a first RNA comprising the nucleotide sequence of SEQ ID NO: 5 and a second RNA comprising the nucleotide sequence of SEQ ID NO: 7, wherein the first RNA and the second RNA are administered at a (w/w) ratio of first RNA to second RNA of about 1.5:1.

In some embodiments of all aspects, the RNA is mRNA.

In some embodiments of all aspects, the RNA is formulated as a liquid, formulated as a solid, or a combination thereof.

In some embodiments of all aspects, the RNA is administered by injection.

In some embodiments of all aspects, the RNA is administered once weekly.

In some embodiments of all aspects, the RNA is administered by intravenous administration.

In some embodiments of all aspects, the RNA is formulated as particles.

In some embodiments, the particles are lipid nanoparticles (LNP).

In some embodiments, the LNP particles comprise ((3-hydroxypropyl)azanediyl)bis(nonane-9,1-diyl) bis(2-butyloctanoate), 2-[(polyethylene glycol)-2000]-N,N-ditetradecylacetamide, 1,2-Distearoyl-sn-glycero-3-phosphocholine, and cholesterol.

In some embodiments of all aspects, the RNA is formulated in a pharmaceutical composition.

In some embodiments, the pharmaceutical composition further comprises one or more pharmaceutically acceptable carriers, diluents and/or excipients.

In some embodiments of all aspects, the method described herein further comprises administering a further therapy.

In some embodiments, the further therapy comprises one or more selected from the group consisting of: (i) surgery to excise, resect, or debulk a tumor, (ii) radiotherapy, and (iii) chemotherapy.

In some embodiments, the further therapy comprises administering a further therapeutic agent.

In some embodiments, the further therapeutic agent comprises an anti-cancer therapeutic agent.

In some embodiments of all aspects, the subject is a human.

In some embodiments of all aspects, the cancer is CLDN6-positive cancer.

In one aspect, the present invention provides a composition or medical preparation described herein for use in a method described herein.

In one aspect, the invention relates to an agent or composition described herein for use in a method described herein.

### BRIEF DESCRIPTION OF THE DRAWINGS

FIG. 1: General structure of the RNA drug substance BNT142

Schematic illustration of the general structure of the RNA drug substance with 5′-cap (here “Cap 1”), 5′- and 3′-UTRs.

C_{H}=constant heavy chain domain; C_{L}=constant light chain domain; DS=drug substance; L=linker; m=messenger; Sec=secretory signal peptide sequence; Poly(A)=poly adenine tail; RNA=ribonucleic acid; UTR=untranslated region; V_{H}=variable heavy chain domain; V_{L}=variable light chain domain.

FIG. 2: RiboMab02.1 binds specifically to human CD3 and CLDN6 and exhibits no off-target binding to the closely related CLDN3, 4 and 9 or non-human primate CD3

Targeted binding of RiboMab02.1 was determined by flow cytometric binding assays using an APC-labeled goat anti-mouse IgG (heavy and light chain [H+L]) secondary antibody. Cells were first gated for singlets followed by gating for viable lymphocytes (PBMCs) or viable HEK-293T-17 cells. RiboMab02.1 in HEK-293T-17 supernatant at a concentration of 10 μg/mL was used. Cynomolgus monkey or human PBMCs and HEK-293T-17 transductants stably expressing luciferase (HEK-293T-17_mock), CLDN3, 4, 6 or 9 served as target cells as indicated. The dotted vertical line marks the peak position of unstained populations.

APC=allophycocyanin; CLDN=claudin; HEK=human embryonic kidney; PBMC=peripheral blood mononuclear cell.

FIG. 3: RiboMab02.1 expressed in mice mediates dose-dependent and target-specific tumor cell lysis in vitro with multiple PBMC donors

Human PBMCs from eight healthy donors were co-cultured with luciferase-expressing tumor cells. CLDN6-positive PA-1 (A) or OV-90 (B) cell lines were used as target cells and the CLDN6-negative MDA-MB-231 (A, B) cell line to control for target specificity. The assays were performed in a 384-well plate format with an effector-to-target-cell ratio of 20:1. Bioluminescence of viable tumor cells was measured as readout. Specific lysis percentages of tumor cell killing are presented. RiboMab02.1-containing mouse serum was serially-diluted (10-fold, 10-point; range: 5.0×10⁻⁷ to 500 ng/mL) and added to the co-cultures, followed by incubation for (A) 24 h with PA-1 cells or (B) 48 h with OV-90 cells. Each line represents tumor cell lysis with an individual donor's PBMC sample. Error bars indicate standard deviation (SD) of the mean (technical triplicates).

CDLN=claudin; EC₅₀=half-maximal effective concentration, PBMC=peripheral blood mononuclear cell.

FIG. 4: RiboMab02.1 induces dose- and target-dependent T-cell proliferation

CFSE-labeled human PBMCs from three healthy donors were co-cultured with CLDN6-positive PA-1 and OV-90 target cells or with CLDN6-negative but control TAA-positive NUGC-4 as well as target-negative MDA-MB-231 control cells in a 12-well culture plate format. An effector-to-target-cell ratio of 10:1 was applied. In addition, PBMCs without (−) target cells were included separately. RiboMab02.1 (1^{st} and 2^{nd} column of each block of five columns)- or control RiboMab (3^{rd} and 4^{th} column of each block of five columns)-containing HEK-293T-17 supernatant at 100 and 1 ng/mL was added to the co-cultures as indicated. OKT3 antibody (anti-human CD3; black bars) served as positive control for target-independent CD3-driven T-cell proliferation. After 72 h of incubation, the percentages of proliferating T cells were analyzed by flow cytometry. Error bars indicate standard deviation (SD) of the mean for all three donors.

CFSE=carboxyfluorescein succinimidyl ester; PBMC=peripheral blood mononuclear cell; w/o=without.

FIG. 5: RiboMab02.1 mediates a dose-dependent T-cell activation with low target-independent effects at high concentrations

Human PBMCs from three healthy donors (effector cells) were cultured in the presence and absence of CLDN6-positive PA-1 cells (target cells) at a 10:1 effector-to-target-cell ratio. RiboMab02.1-containing mouse serum was serially-diluted (10-fold, 10-point; range: 4.0×10⁻⁶ to 4,000 ng/mL) prior to use in the assay. After 48 h of co-incubation, cells were stained with anti-CD5, anti-CD69 and anti-CD25 antibodies for flow cytometric analysis of T cells. Shown here is the total T-cell activation normalized to samples incubated with mock serum from Luc_RNA-LNP-treated mice. Percentages of activated T cells are shown for each individual donor (left) and for all three donors as mean (right). Filled symbols represent values with and black, open symbols without target cells. Error bars indicate standard deviation (SD) of the mean (technical triplicates [per donor, left panel] or biological replicates [across donors, right panel]).

EC₅₀=half-maximal effective concentration; w/o=without.

FIG. 6: BNT142 treatment eliminates advanced xenograft tumors in PBMC-humanized NSG mice by T-cell redirection to the tumor

NSG mice bearing advanced SC tumor xenografts of OV-90 cells (mean tumor volume at treatment start=100 mm³) were engrafted with human PBMCs as effector cells by IP injection. Tumor volume was measured twice per week with a digital caliper. Mice were treated with five IV bolus injections of 0.1 or 1 μg BNT142 or 1 μg of an RNA-LNP encoding a target-irrelevant RiboMab tribody (negative control for target specificity), 1 μg Luc_RNA-LNP, 100 μg of a recombinant purified CD3×(CLDN6)₂ tribody reference protein or DPBS (saline) as vehicle control once weekly. (A) Treatment schedule. (B) Tumor volume of individual mice at designated time points. (C) Overview of the median tumor volume per group, each comprising 13 to 14 mice at the start of the study and a minimum of five mice at the last data points. The vertical dotted lines represent the time points of IV administration of test/control items. Four mice (five mice from the 0.1 μg BNT142 group) from all groups were euthanized on Day 38 to obtain samples for ex vivo assays. (D) Number of CD3-positive cells per mm² of xenograft tissue as determined by immunohistochemical (IHC) staining using an anti-human CD3 antibody. Tumor xenografts were dissected 72 h after the third treatment (Day 38). Horizontal lines represent the mean (n=4 to 5). (E) Percentage of CLDN6-positive cells in tumor xenografts of mice from the respective test and control groups as determined by IHC staining using an anti-human CLDN6 antibody. Tumor xenografts were dissected at different time points over the course of the study. Horizontal lines represent the mean (n=8 to 10). (F) Representative IHC photographs of human CD3 (top panel) and CLDN6 (bottom panel) staining in OV-90 tumor xenografts from BNT142-treated as well as control mice euthanized 72 h after the third treatment (Day 38). Reddish-brown staining (dark in the black and white figure) indicates a positive IHC signal while the bluish-purple areas indicate an absence of positive staining (negative IHC signal). Length of scale bars are as indicated in the panels (BNT142 and reference protein groups: 1,000 μm; negative control/Luc_RNA-LNP and saline groups: 2,000 am).

CD=cluster of differentiation; CLDN=claudin; ctrl=control; IP=intraperitoneal; IV=intravenous; LNP=lipid nanoparticle; Luc=luciferase encoding; neg.=negative; NSG=NOD.Cg-Prkd^{scid} IL2rg^{tm1Wjl}/SzJ; PBMC=peripheral blood mononuclear cell; RNA=ribonucleic acid; SC=subcutaneous.

FIG. 7: RiboMab02.1 induces human cytokines in a dose- and target-dependent manner Cell culture supernatants from the T-cell activation assay (see above, FIG. 5) were used for determining human cytokine (IFN-γ, TNF-α, IL-6, IL-2, IL-10 and IL-1β) production driven by different concentrations of RiboMab02.1 with a customized multiplex ELISA kit. Cytokine concentration values for each donor (means of technical triplicates) are shown. Filled symbols represent values with target cells while open symbols represent values without target cells.

IFN=interferon; IL=interleukin; TNF=tumor necrosis factor; w/o=without.

FIG. 8: BNT142-treatment does not induce human cytokine release in PBMC-humanized NSG mice

Serum from NSG/PBMC mice bearing a subcutaneous xenograft tumor (see above, FIG. 6) was further assessed 6 and 72 h after the third injection with 0.1 or 1 μg BNT142, 1 μg of an RNA-LNP encoding a target-irrelevant RiboMab tribody to control for target specificity (neg. ctrl), 1 μg Luc_RNA-LNP control or 100 μg CD3×(CLDN6)₂ tribody reference protein. An additional non-tumor-bearing (w/o tumor) group administered with 1 μg BNT142 was included. (A) Concentrations of human cytokines (IFN-γ, IL-6, IL-2, IL-10, TNF-α and IL-1β) in mouse serum as determined by multiplex ELISA at the 6- (n=8) and 72-hour (n=4) time points. Data was normalized to saline-administered animals of the respective time points. Horizontal lines indicate medians. Unpaired and paired samples were compared using the Mann-Whitney U test or Wilcoxon signed-rank test, respectively. (B) Serum concentration of the encoded therapeutic antibody RiboMab02.1. Horizontal lines indicate means.

*** p=0.0002; ctrl=control; h=hours; IFN=interferon; IL=interleukin; LNP=lipid nanoparticle; Luc=luciferase encoding; neg.=negative; ns=not significant; RNA=ribonucleic acid; TNF=tumor necrosis factor; w/o=without.

FIG. 9: Liver targeting of LNP-formulated mRNA in vivo

Balb/cJRj mice received a single IV injection of LNP-formulated firefly luciferase mRNA. Bioluminescence was monitored 6, 24, 48, 72, and 144 h after administration. (A) Images taken 6 h post-administration are shown for (left) individual mice in the ventral position (n=5) and (right) single organs of animals #1 and 2. (B) Quantification of luciferase signals (photons/second) is shown for all time points of analysis (n=5 or 3, mean).

IV=intravenous; LN=lymph nodes; LNP=lipid nanoparticle.

FIG. 10: RiboMab02.1 encoded by BNT142 is efficiently expressed in vivo

Female Balb/cJRj mice (n=3) received an IV bolus injection of 30 μg BNT142 per mouse. Serum was harvested 2 and 6 h post-administration. (A) Quantification of RiboMab02.1 concentrations in serum by ELISA. Horizontal lines represent the mean. (B) Western blot analysis of RiboMab02.1-containing serum and reference protein (monomer, HMW) in buffer or spiked-in Balb/cJRj serum were analyzed under non-reducing conditions. In total, 60 ng protein per lane was loaded after a serum-protein purification step. Serum from an untreated mouse served as control. Western blot was performed using a horseradish peroxidase (HRP)-conjugated goat-anti-human IgG Fd antibody.

Ab=antibody; ctrl=control; Fd=fragment detectable; HMW=high molecular weight; HPI=hours post injection; ID=identification number; IgG=immunoglobulin gamma; kDa=kilodalton; MW=molecular weight.

FIG. 11: Sustainable RiboMab02.1 exposure and dose-dependent anti-drug antibody responses by repeated BNT142 dosing of mice

Female Balb/cJRj mice (n=4) were injected IV with 10 or 30 μg BNT142 or 30 μg Luc_RNA-LNP as control once weekly for a total of five administrations at the time points indicated by the horizontal dotted lines. Blood was drawn from mice at baseline (0 h), 6 h after (6, 174, 342, 510, and 678 h) and 24 h before (144, 312, 480, and 648 h) each BNT142 or saline administration, respectively. A final blood draw was done 816 h after the first BNT142/saline administration. Serum RiboMab02.1 concentrations were determined by ELISA. Error bars indicate standard deviation (SD) of the mean.

LNP=lipid nanoparticle; Luc=luciferase; RNA=ribonucleic acid.

FIG. 12: RiboMab02.1 exposure in cynomolgus monkeys after IV injection of BNT142

The BNT142 dose cohorts and saline control group each comprised three cynomolgus monkeys, from whom blood was drawn to prepare serum for assessing RiboMab02.1 concentration by ELISA. Error bars indicate standard deviation (SD) of the mean (technical triplicates).

FIG. 13: RiboMab02.1 is highly monomeric and induces lower ADA response in mice than the alternative lead structure candidate RiboMab_712/711 C53W

Female Balb/cJRj were injected IV with 30 μg RNA-LNP encoding RiboMab02.1 or RiboMab_712/711 or luciferase (control). (A) Serum was sampled 6 hours post-injection. 50 ng purified protein references (monomer and HMW reference) were spiked in mouse serum. 5 μL serum of untreated (mock), luciferase RNA injected (control) or RiboMab RNA injected mice and the spiked references were subjected to Melon G-purification and separated under non-reducing conditions on 4-15% Criterion gels. Western blot analysis was performed with an HRP-conjugated goat anti-human IgG Fd antibody. Samples of one representative mouse per group are shown. (B) For ADA analysis serum was sampled at the indicated time points. Serum samples were analyzed for anti-RiboMab ADA content via a sandwich ELISA assay. ADA response (black lines) are plotted against RiboMab protein concentration (grey dotted line) over time. RiboMab_712/711 C53W variant (top) and RiboMab02.1 (bottom) are shown. Error bars show standard deviation of the mean (n=4).

Ab=antibody; ADA=anti-drug antibodies; C53S/W=cysteine to serine/tryptohpane substitution at position 53 in the anti-CLDN6 VL moiety; Fd=fragment difficult; HMW=high molecular weight; IgG=immunoglobulin G; kDa=kilo Dalton; MW=molecular weight; RU=relative units.

FIG. 14: The HC:LC weight ratio of RiboMab02.1-encoding drug substance intermediates affects the expression efficiency and monomer content of RiboMab02.1

HEK-293T-17 cells were electroporated with the indicated weight (w/w) ratios of the two RiboMab02.1-encoding drug substance intermediates (RNAs) encoding the RiboMab02.1 heavy chain (HC) and light chain (LC), respectively. Cell culture supernatant (SN) was harvested 48 h post-transfection. (A) Western blot analysis of RiboMab02.1-containing SN and reference protein (monomer, HMW) under non-reducing conditions. SN from non-transfected cells served as negative control (mock SN). Western blot was performed using a combination of HRP-conjugated goat-anti-human kappa light chain (1:500) and IgG Fd (1:2,000) antibodies for detection. (B) Mean RiboMab02.1 concentration in SN samples of technical duplicates from two independent experiments was analyzed by ELISA. Error bars indicate standard deviation (SD) of the mean. Ab=antibody; Fd=fragment detectable; HC=heavy chain-encoding RNA; HMW=high molecular weight; IgG=immunoglobulin gamma; kDa=kilodaltons; MW=molecular weight; LC=light chain-encoding RNA; LMW=low molecular weight; RNA=ribonucleic acid; SN=supernatant.

### DESCRIPTION OF THE SEQUENCES

The following table provides a listing of certain sequences referenced herein.

DESCRIPTION OF SEQUENCES

| | | |
|---|---|---|
| SEQ | | |
| ID | | |
| NO: | Description | SEQUENCE |

Claudin-6 (CLDN6)

| | |
|---|---|
| 1 | MASAGMQILGVVLTLLGWVNGLVSCALPMWKVTAFIGNSIVVAQVVWEGLWMSCVVQSTGQMQCKVYDSLLALPQDLQAARALCVIALLVAL |
| | FGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQ |
| | GPSHYMARYSTSAPAISRGPSEYPTKNYV |
| 2 | MASAGMQILGVVLTLLGWVNGLVSCALPMWKVTAFIGNSIVVAQVVWEGLWMSCVVQSTGQMQCKVYDSLLALPQDLQAARALCVIALLVAL |
| | FGLLVYLAGAKCTTCVEEKDSKARLVLTSGIVFVISGVLTLIPVCWTAHAVIRDFYNPLVAEAQKRELGASLYLGWAASGLLLLGGGLLCCTCPSGGSQ |
| | GPSHYMARYSTSAPAISRGPSEYPTKNYV |

CD3 Epsilon

| | |
|---|---|
| 3 | MQSGTHWRVLGLCLLSVGVWGQDGNEEMGGITQTPYKVSISGTTVILTCPQYPGSEILWQHNDKNIGGDEDDKNIGSDEDHLSLKEFSELEQSGY |
| | YVCYPRGSKPEDANFYLYLRARVCENCMEMDVMSVATIVIVDICITGGLLLLVYYWSKNRKAKAKPVTRGAGAGGRQRGQNKERPPPVPNPDYE |
| | PIRKGQRDLYSGLNQRRI |

RiboMab02.1 HC-First polypeptide (Fd)

| | | |
|---|---|---|
| 4 | Amino acid | MRVMAPRTLILLLSGALALTETWAGSQVQLQQSGAELARPGASVKMSCKTSGYTFTRYTMHWVKQRPGQGLEWIGYINPSRGYTNYNQKFKDK |
| | sequence | ATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSG |
| | | ALTSGVHTFPAVLQSSGLYSLSSVVTVPSSSLGTQTYICNVNHKPSNTKVDKRVEPKSCSGPGGGRSGGGGSGGGGSEVQLQQSGPELVKPGASM |
| | | KISCKASGYSFTGYTMNWVKQSHGKCLEWIGLINPYNGGTIYNQKFKGKATLTVDKSSSTAYMELLSLTSEDSAVYYCARDYGFVLDYWGQGTTLT |
| | | VSSGGGGSGGGGSGGGGSGGGGSQIVLTQSPSIMSVSPGEKVTITCSASSSVSYMHWFQQKPGTSPKLSIYSTSNLASGVPARFSGRGSGTSYSLTI |
| | | SRVAAEDAATYYCQQRSNYPPWTFGCGTKLEIK |
| 5 | mRNA sequence | AGAAUAAACUAGUAUUCUUCUGGUCCCCACAGACUCAGAGAGAACCCGCCACCAUGAGAGUGAUGGCCCCUAGAACACUGAUCCUGCU |
| | | GCUGUCUGGUGCCCUGGCUCUGACAGAAACAUGGGCCGGAUCUCAGGUGCAGCUCCAGCAAUCUGGUGCCGAACUUGCUAGACCUGG |
| | | CGCCUCCGUGAAGAUGAGCUGUAAAACCAGCGGCUACACCUUCACACGGUACACCAUGCACUGGGUCAAGCAGAGGCCUGGACAGGGC |
| | | CUUGAGUGGAUCGGCUACAUCAACCCCAGCCGGGGCUACACCAACUACAACCAGAAGUUCAAGGACAAGGCCACACUGACCACCGACAA |
| | | GAGCAGCAGCACAGCCUACAUGCAGCUGAGCAGCCUGACCAGCGAAGAUAGCGCCGUGUACUACUGCGCCCGGUACUACGACGAUCAC |
| | | UACAGCCUGGAUUACUGGGGCCAGGGAACAACCCUGACAGUGUCUAGCGCCAGCACCAAGGGACCUAGCGUUUUCCCACUGGCUCCCA |
| | | GCAGCAAGAGCACAUCUGGUGGAACAGCCGCUCUGGGCUGCCUGGUCAAGGAUUACUUUCCCGAGCCUGUGACCGUGUCCUGGAAUU |
| | | CUGGCGCUCUGACAAGCGGCGUGCACACCUUUCCAGCCGUGCUGCAAAGCAGCGGCCUGUACUCUCUGAGCAGCGUGGUCACAGUGCC |
| | | UAGCUCUAGCCUGGGCACCCAGACCUACAUCUGCAAUGUGAACCACAAGCCUAGCAACACCAAGGUGGACAAGAGAGUGGAACCCAAG |
| | | AGCUGUUCUGGACCCGGCGGAGGAAGAUCUGGCGGAGGCGGUUCUGGUGGCGGAGGAUCUGAAGUUCAGCUGCAACAGUCUGGCCC |
| | | CGAGCUGGUUAAGCCUGGGGCCUCUAUGAAGAUCUCCUGCAAGGCCUCCGGCUACAGCUUUACCGGCUACACAAUGAAUUGGGUUAA |
| | | GCAGUCCCACGGCAAGUGCCUGGAAUGGAUCGGCCUGAUCAACCCUUACAACGGCGGCACCAUCUAUAAUCAGAAGUUUAAAGGCAAG |
| | | GCUACCCUCACCGUGGACAAGUCUAGCUCCACCGCCUACAUGGAACUGCUGAGCCUGACCUCUGAGGACUCCGCCGUGUAUUAUUGUG |
| | | CCAGAGACUACGGCUUCGUGCUGGACUAUUGGGGACAGGGCACUACACUGACUGUGUCCAGUGGCGGUGGUGGCAGUGGCGGCGGA |
| | | GGUAGCGGAGGUGGUGGAAGCGGAGGCGGAGGCUCUCAAAUUGUGCUGACACAGAGCCCCAGCAUCAUGAGCGUUAGCCCUGGCGAG |
| | | AAAGUGACCAUCACAUGCAGCGCCAGCUCCUCCGUGUCCUAUAUGCACUGGUUUCAGCAGAAGCCCGGCACAAGCCCCAAGCUGUCGA |
| | | UCUACAGCACCAGCAACCUGGCCAGCGGAGUGCCUGCCAGAUUUUCUGGUAGAGGCAGCGGCACCAGCUACUCCCUGACAAUCUCUAG |
| | | AGUGGCCGCCGAAGAUGCCGCCACCUACUACUGUCAGCAGCGGAGCAAUUACCCUCCUUGGACCUUUGGCUGCGGCACCAAGCUGGAA |
| | | AUCAAGUGAUGAGGAUCCGAUCUGGUACUGCAUGCACGCAAUGCUAGCUGCCCCUUUCCCGUCCUGGGUACCCCGAGUCUCCCCCGAC |
| | | CUCGGGUCCCAGGUAUGCUCCCACCUCCACCUGCCCCACUCACCACCUCUGCUAGUUCCAGACACCUCCCAAGCACGCAGCAAUGCAGCU |
| | | CAAAACGCUUAGCCUAGCCACACCCCCACGGGAAACAGCAGUGAUUAACCUUUAGCAAUAAACGAAAGUUUAACUAAGCUAUACUAAC |
| | | CCCAGGGUUGGUCAAUUUCGUGCCAGCCACACCCUCGAGCUAGCAAAAAAAAAAAAAAAAAAAAAAAAAAAAAAGCAUAUGACUAAAA |
| | | AAAAAAAAAAAAAAAAAAAAAAAAAAAAAAAAAAAAAAAAAAAAAAAAAAAAAAAAAAAAAAAAAA |

RiboMab02.1 LC-Second polypeptide (L)

| | | |
|---|---|---|
| 6 | Amino acid | MRVMAPRTLILLLSGALALTETWAGSQIVLTQSPAIMSASPGEKVTMTCRASSSVSYMNWYQQKSGTSPKRWIYDTSKVASGVPYRFSGSGSGTS |
| | sequence | YSLTISSMEAEDAATYYCQQWSSNPLTFGAGTKLELKRTVAAPSVFIFPPSDEQLKSGTASVVCLLNNFYPREAKVQWKVDNALQSGNSQESVTEQ |
| | | DSKDSTYSLSSTLTLSKADYEKHKVYACEVTHQGLSSPVTKSFNRGECDVPGGSEVQLQQSGPELVKPGASMKISCKASGYSFTGYTMNWVKQSH |
| | | GKCLEWIGLINPYNGGTIYNQKFKGKATLTVDKSSSTAYMELLSLTSEDSAVYYCARDYGFVLDYWGQGTTLTVSSGGGGSGGGGSGGGGSGGGG |
| | | SQIVLTQSPSIMSVSPGEKVTITCSASSSVSYMHWFQQKPGTSPKLSIYSTSNLASGVPARFSGRGSGTSYSLTISRVAAEDAATYYCQQRSNYPPW |
| | | TFGCGTKLEIK |
| 7 | mRNA sequence | AGAAUAAACUAGUAUUCUUCUGGUCCCCACAGACUCAGAGAGAACCCGCCACCAUGAGAGUGAUGGCCCCUAGAACACUGAUCCUGCU |
| | | GCUGUCUGGUGCCCUGGCUCUGACAGAAACAUGGGCCGGAUCUCAGAUCGUGCUGACACAGAGCCCUGCCAUCAUGAGUGCCUCUCCA |
| | | GGCGAGAAAGUGACCAUGACCUGUAGAGCCAGCAGCAGCGUGUCCUACAUGAACUGGUAUCAGCAGAAGUCCGGCACAAGCCCCAAGC |
| | | GGUGGAUCUACGAUACAAGCAAGGUGGCCAGCGGCGUGCCCUACAGAUUUUCUGGCUCUGGCAGCGGCACCAGCUACAGCCUGACAA |
| | | UCAGCAGCAUGGAAGCCGAGGAUGCCGCCACCUACUACUGCCAGCAGUGGUCCAGCAAUCCCCUGACAUUUGGAGCCGGCACCAAGCU |
| | | GGAACUGAAGCGGACAGUUGCCGCUCCUAGCGUGUUCAUCUUCCCACCUUCCGACGAGCAGCUGAAGUCUGGAACAGCCAGCGUCGUG |
| | | UGCCUGCUGAACAACUUCUACCCUCGGGAAGCCAAGGUGCAGUGGAAGGUGGACAAUGCCCUCCAGUCCGGCAACAGCCAAGAGAGCG |
| | | UGACCGAGCAGGACAGCAAGGACUCCACCUAUAGCCUGAGCAGCACCCUGACACUGAGCAAGGCCGACUACGAGAAACACAAGGUGUA |
| | | CGCCUGCGAAGUGACCCACCAGGGACUGUCUAGCCCUGUGACCAAGAGCUUCAACAGAGGCGAGUGUGAUGUGCCUGGCGGCUCUGA |
| | | AGUUCAGCUCCAGCAGUCUGGACCCGAGCUGGUUAAGCCUGGCGCCUCCAUGAAGAUCUCUUGCAAGGCCUCCGGCUACAGCUUCACC |
| | | GGCUACACCAUGAAUUGGGUCAAGCAGAGCCACGGCAAGUGCCUGGAAUGGAUCGGCCUGAUCAACCCCUACAACGGCGGCACAAUCU |
| | | ACAACCAGAAGUUCAAGGGCAAAGCCACACUGACCGUGGACAAGAGCAGCAGCACCGCCUAUAUGGAACUGCUGAGCCUGACCAGCGA |
| | | GGACUCCGCCGUGUACUACUGCGCCAGAGAUUACGGCUUCGUGCUGGACUAUUGGGGCCAGGGAACAACCCUGACAGUGUCUAGCGG |
| | | AGGCGGAGGAUCUGGUGGCGGAGGAAGUGGCGGAGGCGGUUCUGGCGGUGGUGGAUCUCAAAUUGUCCUGACUCAGUCCCCUAGCA |
| | | UCAUGAGCGUGUCACCCGGGGAGAAAGUGACAAUCACCUGUUCCGCCAGCUCCUCCGUGUCCUACAUGCACUGGUUCCAGCAGAAGCC |
| | | CGGCACCUCCCCCAAGCUGUCCAUCUACUCCACCUCCAACCUGGCCUCCGGCGUGCCCGCCAGAUUCUCUGGCAGAGGCUCCGGCACCAG |
| | | CUACUCCCUGACCAUCUCUAGAGUGGCCGCCGAGGACGCUGCCACAUAUUAUUGUCAGCAGCGGAGCAACUACCCUCCUUGGACCUUU |
| | | GGCUGCGGAACAAAGCUGGAAAUCAAGUGAUGAGGAUCCGAUCUGGUACUGCAUGCACGCAAUGCUAGCUGCCCCUUUCCCGUCCUG |
| | | GGUACCCCGAGUCUCCCCCGACCUCGGGUCCCAGGUAUGCUCCCACCUCCACCUGCCCCACUCACCACCUCUGCUAGUUCCAGACACCUC |
| | | CCAAGCACGCAGCAAUGCAGCUCAAAACGCUUAGCCUAGCCACACCCCCACGGGAAACAGCAGUGAUUAACCUUUAGCAAUAAACGAAA |
| | | GUUUAACUAAGCUAUACUAACCCCAGGGUUGGUCAAUUUCGUGCCAGCCACACCCUCGAGCUAGCAAAAAAAAAAAAAAAAAAAAAA |
| | | AAAAAAAAGCAUAUGACUAAAAAAAAAAAAAAAAAAAAAAAAAAAAAAAAAAAAAAAAAAAAAAAAAAAAAAAAAAAAAAAAAAAAAA |

5′-UTR (hAg-Kozak)

| | | |
|---|---|---|
| 8 | 5′-UTR | AACUAGUAUUCUUCUGGUCCCCACAGACUCAGAGAGAACCCGCCACC |

3′-UTR (FI element)

| | | |
|---|---|---|
| 9 | 3′-UTR | CUGGUACUGCAUGCACGCAAUGCUAGCUGCCCCUUUCCCGUCCUGGGUACCCCGAGUCUCCCCCGACCUCGGGUCCCAGGUAUGCUCCC |
| | | ACCUCCACCUGCCCCACUCACCACCUCUGCUAGUUCCAGACACCUCCCAAGCACGCAGCAAUGCAGCUCAAAACGCUUAGCCUAGCCACA |
| | | CCCCCACGGGAAACAGCAGUGAUUAACCUUUAGCAAUAAACGAAAGUUUAACUAAGCUAUACUAACCCCAGGGUUGGUCAAUUUCGU |
| | | GCCAGCCACACC |

A30L70

| | | |
|---|---|---|
| 10 | A30L70 | AAAAAAAAAAAAAAAAAAAAAAAAAAAAAAGCAUAUGACUAAAAAAAAAAAAAAAAAAAAAAAAAAAAAAAAAAAAAAAAAAAAAAA |
| | | AAAAAAAAAAAAAAAAAAAAAAA |

Linkers

| | | |
|---|---|---|
| 11 | (Gly4Ser)2 linker | GGGGSGGGGS |
| 12 | (Gly4Ser)3 linker | GGGGSGGGGSGGGGS |
| 13 | (Gly4Ser)4 linker | GGGGSGGGGSGGGGSGGGGS |
| 14 | (Gly4Ser)5 linker | GGGGSGGGGSGGGGSGGGGSGGGGS |
| 15 | (Gly4Ser)6 linker | GGGGSGGGGSGGGGSGGGGSGGGGSGGGGS |
| 16 | Linker 1 | SGPGGGRSGGGGSGGGGS |
| 17 | Linker 2 | DVPGGS |

CDRs

| | | |
|---|---|---|
| 18 | VH(CD3)-CDR1 | GYTFTRYT |
| 19 | VH(CD3)-CDR2 | INPSRGYT |
| 20 | VH(CD3)-CDR3 | ARYYDDHYSLDY |
| 21 | VH(CD3)-CDR3 | ARYYDDHYCLDY |
| 22 | VL(CD3)-CDR1 | SSVSY |
| 23 | VL(CD3)-CDR2 | DTS |
| 24 | VL(CD3)-CDR3 | QQWSSNPLT |
| 25 | VH(CLDN6)-CDR1 | GYSFTGYT |
| 26 | VH(CLDN6)-CDR2 | INPYNGGT |
| 27 | VH(CLDN6)-CDR3 | ARDYGFVLDY |
| 28 | VL(CLDN6)-CDR1 | SSVSY |
| 29 | VL(CLDN6)-CDR2 | STS |
| 30 | VL(CLDN6)-CDR3 | QQRSNYPPWT |

RiboMab_712/711 HC-First polypeptide (Fd)

| | | |
|---|---|---|
| 31 | Amino acid | MRVMAPRTLILLLSGALALTETWAGSQVQLQQSGAELARPGASVKMSCKTSGYTFTRYTMHWVKQRPGQGLEWIGYINPSRGYTNYNQKFKDK |
| | sequence | ATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSG |
| | | ALTSGVHTFPAVLQSSGLYSLSSVVTVPSSSLGTQTYICNVNHKPSNTKVDKRVEPKSCSGPGGGRSGGGGSGGGGSEVQLQQSGPELVKPGASM |
| | | KISCKASGYSFTGYTMNWVKQSHGKCLEWIGLINPYNGGTIYNQKFKGKATLTVDKSSSTAYMELLSLTSEDSAVYYCARDYGFVLDYWGQGTTLT |
| | | VSSGGGGSGGGGSGGGGSGGGGSQIVLTQSPSIMSVSPGEKVTITCSASSSVSYMHWFQQKPGTSPKLWIYSTSNLASGVPARFSGRGSGTSYSL |
| 32 | mRNA sequence | AGAAUAAACUAGUAUUCUUCUGGUCCCCACAGACUCAGAGAGAACCCGCCACCAUGAGAGUGAUGGCCCCUAGAACACUGAUCCUGCU |
| | | GCUGUCUGGUGCCCUGGCUCUGACAGAAACAUGGGCCGGAUCUCAGGUGCAGCUCCAGCAAUCUGGUGCCGAACUUGCUAGACCUGG |
| | | CGCCUCCGUGAAGAUGAGCUGUAAAACCAGCGGCUACACCUUCACACGGUACACCAUGCACUGGGUCAAGCAGAGGCCUGGACAGGGC |
| | | CUUGAGUGGAUCGGCUACAUCAACCCCAGCCGGGGCUACACCAACUACAACCAGAAGUUCAAGGACAAGGCCACACUGACCACCGACAA |
| | | GAGCAGCAGCACAGCCUACAUGCAGCUGAGCAGCCUGACCAGCGAAGAUAGCGCCGUGUACUACUGCGCCCGGUACUACGACGAUCAC |
| | | UACAGCCUGGAUUACUGGGGCCAGGGAACAACCCUGACAGUGUCUAGCGCCAGCACCAAGGGACCUAGCGUUUUCCCACUGGCUCCCA |
| | | GCAGCAAGAGCACAUCUGGUGGAACAGCCGCUCUGGGCUGCCUGGUCAAGGAUUACUUUCCCGAGCCUGUGACCGUGUCCUGGAAUU |
| | | CUGGCGCUCUGACAAGCGGCGUGCACACCUUUCCAGCCGUGCUGCAAAGCAGCGGCCUGUACUCUCUGAGCAGCGUGGUCACAGUGCC |
| | | UAGCUCUAGCCUGGGCACCCAGACCUACAUCUGCAAUGUGAACCACAAGCCUAGCAACACCAAGGUGGACAAGAGAGUGGAACCCAAG |
| | | AGCUGUUCUGGACCCGGCGGAGGAAGAUCUGGCGGAGGCGGUUCUGGUGGCGGAGGAUCUGAAGUUCAGCUGCAACAGUCUGGCCC |
| | | CGAGCUGGUUAAGCCUGGGGCCUCUAUGAAGAUCUCCUGCAAGGCCUCCGGCUACAGCUUUACCGGCUACACAAUGAAUUGGGUUAA |
| | | GCAGUCCCACGGCAAGUGCCUGGAAUGGAUCGGCCUGAUCAACCCUUACAACGGCGGCACCAUCUAUAAUCAGAAGUUUAAAGGCAAG |
| | | GCUACCCUCACCGUGGACAAGUCUAGCUCCACCGCCUACAUGGAACUGCUGAGCCUGACCUCUGAGGACUCCGCCGUGUAUUAUUGUG |
| | | CCAGAGACUACGGCUUCGUGCUGGACUAUUGGGGACAGGGCACUACACUGACUGUGUCCAGUGGCGGUGGUGGCAGUGGCGGCGGA |
| | | GGUAGCGGAGGUGGUGGAAGCGGAGGCGGAGGCUCUCAAAUUGUGCUGACACAGAGCCCCAGCAUCAUGAGCGUUAGCCCUGGCGAG |
| | | AAAGUGACCAUCACAUGCAGCGCCAGCUCCUCCGUGUCCUAUAUGCACUGGUUUCAGCAGAAGCCCGGCACAAGCCCCAAGCUGUGGA |
| | | UCUACAGCACCAGCAACCUGGCCAGCGGAGUGCCUGCCAGAUUUUCUGGUAGAGGCAGCGGCACCAGCUACUCCCUGACAAUCUCUAG |
| | | AGUGGCCGCCGAAGAUGCCGCCACCUACUACUGUCAGCAGCGGAGCAAUUACCCUCCUUGGACCUUUGGCUGCGGCACCAAGCUGGAA |
| | | AUCAAGUGAUGAGGAUCCGAUCUGGUACUGCAUGCACGCAAUGCUAGCUGCCCCUUUCCCGUCCUGGGUACCCCGAGUCUCCCCCGAC |
| | | CUCGGGUCCCAGGUAUGCUCCCACCUCCACCUGCCCCACUCACCACCUCUGCUAGUUCCAGACACCUCCCAAGCACGCAGCAAUGCAGCU |
| | | CAAAACGCUUAGCCUAGCCACACCCCCACGGGAAACAGCAGUGAUUAACCUUUAGCAAUAAACGAAAGUUUAACUAAGCUAUACUAAC |
| | | CCCAGGGUUGGUCAAUUUCGUGCCAGCCACACCCUCGAGCUAGCAAAAAAAAAAAAAAAAAAAAAAAAAAAAAAGCAUAUGACUAAAA |
| | | AAAAAAAAAAAAAAAAAAAAAAAAAAAAAAAAAAAAAAAAAAAAAAAAAAAAAAAAAAAAAAAAAA |

RiboMab_712/711 LC-Second polypeptide (L)

| | | |
|---|---|---|
| 33 | Amino acid | MRVMAPRTLILLLSGALALTETWAGSQIVLTQSPAIMSASPGEKVTMTCRASSSVSYMNWYQQKSGTSPKRWIYDTSKVASGVPYRFSGSGSGTS |
| | sequence | YSLTISSMEAEDAATYYCQQWSSNPLTFGAGTKLELKRTVAAPSVFIFPPSDEQLKSGTASVVCLLNNFYPREAKVQWKVDNALQSGNSQESVTEQ |
| | | DSKDSTYSLSSTLTLSKADYEKHKVYACEVTHQGLSSPVTKSFNRGECDVPGGSEVQLQQSGPELVKPGASMKISCKASGYSFTGYTMNWVKQSH |
| | | GKCLEWIGLINPYNGGTIYNQKFKGKATLTVDKSSSTAYMELLSLTSEDSAVYYCARDYGFVLDYWGQGTTLTVSSGGGGSGGGGSGGGGSGGGG |
| | | SQIVLTQSPSIMSVSPGEKVTITCSASSSVSYMHWFQQKPGTSPKLWIYSTSNLASGVPARFSGRGSGTSYSLTISRVAAEDAATYYCQQRSNYPPW |
| | | TFGCGTKLEIK |
| 34 | mRNA sequence | AGAAUAAACUAGUAUUCUUCUGGUCCCCACAGACUCAGAGAGAACCCGCCACCAUGAGAGUGAUGGCCCCUAGAACACUGAUCCUGCU |
| | | GCUGUCUGGUGCCCUGGCUCUGACAGAAACAUGGGCCGGAUCUCAGAUCGUGCUGACACAGAGCCCUGCCAUCAUGAGUGCCUCUCCA |
| | | GGCGAGAAAGUGACCAUGACCUGUAGAGCCAGCAGCAGCGUGUCCUACAUGAACUGGUAUCAGCAGAAGUCCGGCACAAGCCCCAAGC |
| | | GGUGGAUCUACGAUACAAGCAAGGUGGCCAGCGGCGUGCCCUACAGAUUUUCUGGCUCUGGCAGCGGCACCAGCUACAGCCUGACAA |
| | | UCAGCAGCAUGGAAGCCGAGGAUGCCGCCACCUACUACUGCCAGCAGUGGUCCAGCAAUCCCCUGACAUUUGGAGCCGGCACCAAGCU |
| | | GGAACUGAAGCGGACAGUUGCCGCUCCUAGCGUGUUCAUCUUCCCACCUUCCGACGAGCAGCUGAAGUCUGGAACAGCCAGCGUCGUG |
| | | UGCCUGCUGAACAACUUCUACCCUCGGGAAGCCAAGGUGCAGUGGAAGGUGGACAAUGCCCUCCAGUCCGGCAACAGCCAAGAGAGCG |
| | | UGACCGAGCAGGACAGCAAGGACUCCACCUAUAGCCUGAGCAGCACCCUGACACUGAGCAAGGCCGACUACGAGAAACACAAGGUGUA |
| | | CGCCUGCGAAGUGACCCACCAGGGACUGUCUAGCCCUGUGACCAAGAGCUUCAACAGAGGCGAGUGUGAUGUGCCUGGCGGCUCUGA |
| | | AGUUCAGCUCCAGCAGUCUGGACCCGAGCUGGUUAAGCCUGGCGCCUCCAUGAAGAUCUCUUGCAAGGCCUCCGGCUACAGCUUCACC |
| | | GGCUACACCAUGAAUUGGGUCAAGCAGAGCCACGGCAAGUGCCUGGAAUGGAUCGGCCUGAUCAACCCCUACAACGGCGGCACAAUCU |
| | | ACAACCAGAAGUUCAAGGGCAAAGCCACACUGACCGUGGACAAGAGCAGCAGCACCGCCUAUAUGGAACUGCUGAGCCUGACCAGCGA |
| | | GGACUCCGCCGUGUACUACUGCGCCAGAGAUUACGGCUUCGUGCUGGACUAUUGGGGCCAGGGAACAACCCUGACAGUGUCUAGCGG |
| | | AGGCGGAGGAUCUGGUGGCGGAGGAAGUGGCGGAGGCGGUUCUGGCGGUGGUGGAUCUCAAAUUGUCCUGACUCAGUCCCCUAGCA |
| | | UCAUGAGCGUGUCACCCGGGGAAAAAGUGACAAUCACAUGCAGCGCCAGCUCCUCCGUGUCUUAUAUGCACUGGUUCCAGCAAAAGCC |
| | | AGGGACCUCUCCUAAGCUCUGGAUCUACAGCACCAGCAACCUGGCCUCUGGCGUGCCAGCUAGAUUUUCCGGUAGAGGCUCCGGCACC |
| | | UCUUACUCCCUGACCAUCUCUAGAGUGGCCGCCGAGGACGCUGCCACAUAUUAUUGUCAGCAGCGGAGCAACUACCCUCCUUGGACCU |
| | | UUGGCUGCGGAACAAAGCUGGAAAUCAAGUGAUGAGGAUCCGAUCUGGUACUGCAUGCACGCAAUGCUAGCUGCCCCUUUCCCGUCC |
| | | UGGGUACCCCGAGUCUCCCCCGACCUCGGGUCCCAGGUAUGCUCCCACCUCCACCUGCCCCACUCACCACCUCUGCUAGUUCCAGACACC |
| | | UCCCAAGCACGCAGCAAUGCAGCUCAAAACGCUUAGCCUAGCCACACCCCCACGGGAAACAGCAGUGAUUAACCUUUAGCAAUAAACGA |
| | | AAGUUUAACUAAGCUAUACUAACCCCAGGGUUGGUCAAUUUCGUGCCAGCCACACCCUCGAGCUAGCAAAAAAAAAAAAAAAAAAAA |
| | | AAAAAAAAAAGCAUAUGACUAAAAAAAAAAAAAAAAAAAAAAAAAAAAAAAAAAAAAAAAAAAAAAAAAAAAAAAAAAAAAAAAAAAAAA |

RiboMab02.1 RNA coding sequences

| | | |
|---|---|---|
| 35 | RiboMab02.1 | AUGAGAGUGAUGGCCCCUAGAACACUGAUCCUGCUGCUGUCUGGUGCCCUGGCUCUGACAGAAACAUGGGCCGGAUCUCAGGUGCAG |
| | HC/First | CUCCAGCAAUCUGGUGCCGAACUUGCUAGACCUGGCGCCUCCGUGAAGAUGAGCUGUAAAACCAGCGGCUACACCUUCACACGGUACA |
| | polypeptide | CCAUGCACUGGGUCAAGCAGAGGCCUGGACAGGGCCUUGAGUGGAUCGGCUACAUCAACCCCAGCCGGGGCUACACCAACUACAACCA |
| | coding sequence | GAAGUUCAAGGACAAGGCCACACUGACCACCGACAAGAGCAGCAGCACAGCCUACAUGCAGCUGAGCAGCCUGACCAGCGAAGAUAGC |
| | (including start | GCCGUGUACUACUGCGCCCGGUACUACGACGAUCACUACAGCCUGGAUUACUGGGGCCAGGGAACAACCCUGACAGUGUCUAGCGCCA |
| | and stop codons) | GCACCAAGGGACCUAGCGUUUUCCCACUGGCUCCCAGCAGCAAGAGCACAUCUGGUGGAACAGCCGCUCUGGGCUGCCUGGUCAAGGA |
| | | UUACUUUCCCGAGCCUGUGACCGUGUCCUGGAAUUCUGGCGCUCUGACAAGCGGCGUGCACACCUUUCCAGCCGUGCUGCAAAGCAGC |
| | | GGCCUGUACUCUCUGAGCAGCGUGGUCACAGUGCCUAGCUCUAGCCUGGGCACCCAGACCUACAUCUGCAAUGUGAACCACAAGCCUA |
| | | GCAACACCAAGGUGGACAAGAGAGUGGAACCCAAGAGCUGUUCUGGACCCGGCGGAGGAAGAUCUGGCGGAGGCGGUUCUGGUGGCG |
| | | GAGGAUCUGAAGUUCAGCUGCAACAGUCUGGCCCCGAGCUGGUUAAGCCUGGGGCCUCUAUGAAGAUCUCCUGCAAGGCCUCCGGCU |
| | | ACAGCUUUACCGGCUACACAAUGAAUUGGGUUAAGCAGUCCCACGGCAAGUGCCUGGAAUGGAUCGGCCUGAUCAACCCUUACAACG |
| | | GCGGCACCAUCUAUAAUCAGAAGUUUAAAGGCAAGGCUACCCUCACCGUGGACAAGUCUAGCUCCACCGCCUACAUGGAACUGCUGAG |
| | | CCUGACCUCUGAGGACUCCGCCGUGUAUUAUUGUGCCAGAGACUACGGCUUCGUGCUGGACUAUUGGGGACAGGGCACUACACUGAC |
| | | UGUGUCCAGUGGCGGUGGUGGCAGUGGCGGCGGAGGUAGCGGAGGUGGUGGAAGCGGAGGCGGAGGCUCUCAAAUUGUGCUGACA |
| | | CAGAGCCCCAGCAUCAUGAGCGUUAGCCCUGGCGAGAAAGUGACCAUCACAUGCAGCGCCAGCUCCUCCGUGUCCUAUAUGCACUGGU |
| | | UUCAGCAGAAGCCCGGCACAAGCCCCAAGCUGUCGAUCUACAGCACCAGCAACCUGGCCAGCGGAGUGCCUGCCAGAUUUUCUGGUAG |
| | | AGGCAGCGGCACCAGCUACUCCCUGACAAUCUCUAGAGUGGCCGCCGAAGAUGCCGCCACCUACUACUGUCAGCAGCGGAGCAAUUACC |
| | | CUCCUUGGACCUUUGGCUGCGGCACCAAGCUGGAAAUCAAGUGAUGA |
| 36 | RiboMab02.1 | AUGAGAGUGAUGGCCCCUAGAACACUGAUCCUGCUGCUGUCUGGUGCCCUGGCUCUGACAGAAACAUGGGCCGGAUCUCAGAUCGUG |
| | LC/Second | CUGACACAGAGCCCUGCCAUCAUGAGUGCCUCUCCAGGCGAGAAAGUGACCAUGACCUGUAGAGCCAGCAGCAGCGUGUCCUACAUGA |
| | polypeptide | ACUGGUAUCAGCAGAAGUCCGGCACAAGCCCCAAGCGGUGGAUCUACGAUACAAGCAAGGUGGCCAGCGGCGUGCCCUACAGAUUUUC |
| | coding sequence | UGGCUCUGGCAGCGGCACCAGCUACAGCCUGACAAUCAGCAGCAUGGAAGCCGAGGAUGCCGCCACCUACUACUGCCAGCAGUGGUCC |
| | (including start | AGCAAUCCCCUGACAUUUGGAGCCGGCACCAAGCUGGAACUGAAGCGGACAGUUGCCGCUCCUAGCGUGUUCAUCUUCCCACCUUCCG |
| | and stop codons) | ACGAGCAGCUGAAGUCUGGAACAGCCAGCGUCGUGUGCCUGCUGAACAACUUCUACCCUCGGGAAGCCAAGGUGCAGUGGAAGGUGG |
| | | ACAAUGCCCUCCAGUCCGGCAACAGCCAAGAGAGCGUGACCGAGCAGGACAGCAAGGACUCCACCUAUAGCCUGAGCAGCACCCUGACA |
| | | CUGAGCAAGGCCGACUACGAGAAACACAAGGUGUACGCCUGCGAAGUGACCCACCAGGGACUGUCUAGCCCUGUGACCAAGAGCUUCA |
| | | ACAGAGGCGAGUGUGAUGUGCCUGGCGGCUCUGAAGUUCAGCUCCAGCAGUCUGGACCCGAGCUGGUUAAGCCUGGCGCCUCCAUGA |
| | | AGAUCUCUUGCAAGGCCUCCGGCUACAGCUUCACCGGCUACACCAUGAAUUGGGUCAAGCAGAGCCACGGCAAGUGCCUGGAAUGGA |
| | | UCGGCCUGAUCAACCCCUACAACGGCGGCACAAUCUACAACCAGAAGUUCAAGGGCAAAGCCACACUGACCGUGGACAAGAGCAGCAGC |
| | | ACCGCCUAUAUGGAACUGCUGAGCCUGACCAGCGAGGACUCCGCCGUGUACUACUGCGCCAGAGAUUACGGCUUCGUGCUGGACUAU |
| | | UGGGGCCAGGGAACAACCCUGACAGUGUCUAGCGGAGGCGGAGGAUCUGGUGGCGGAGGAAGUGGCGGAGGCGGUUCUGGCGGUGG |
| | | UGGAUCUCAAAUUGUCCUGACUCAGUCCCCUAGCAUCAUGAGCGUGUCACCCGGGGAGAAAGUGACAAUCACCUGUUCCGCCAGCUCC |
| | | UCCGUGUCCUACAUGCACUGGUUCCAGCAGAAGCCCGGCACCUCCCCCAAGCUGUCCAUCUACUCCACCUCCAACCUGGCCUCCGGCGU |
| | | GCCCGCCAGAUUCUCUGGCAGAGGCUCCGGCACCAGCUACUCCCUGACCAUCUCUAGAGUGGCCGCCGAGGACGCUGCCACAUAUUAU |
| | | UGUCAGCAGCGGAGCAACUACCCUCCUUGGACCUUUGGCUGCGGAACAAAGCUGGAAAUCAAGUGAUGA |

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure. Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of” or “consisting essentially of”. Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

In the following, definitions will be provided which apply to all aspects of the present disclosure. The following terms have the following meanings unless otherwise indicated. Any undefined terms have their art recognized meanings.

Terms such as “reduce”, “decrease”, “inhibit” or “impair” as used herein relate to an overall reduction or the ability to cause an overall reduction, preferably of at least 5%, at least 10%, at least 20%, at least 50%, at least 75% or even more, in the level. These terms include a complete or essentially complete inhibition, i.e., a reduction to zero or essentially to zero.

Terms such as “increase”, “enhance” or “exceed” preferably relate to an increase or enhancement by at least 10%, at least 20%, at least 30%, at least 40%, at least 50%, at least 80%, at least 100%, at least 200%, at least 500%, or even more.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 150 amino acids, but the terms “peptide”, “protein” and “polypeptide” are used herein usually as synonyms.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. In some embodiments, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In some embodiments, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant (e.g., functional variant) of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In some embodiments, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to sequences of binding agents, one particular function is one or more binding activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., binding to a target molecule. In some embodiments, the modifications in the amino acid sequence of the parent molecule or sequence do not significantly affect or alter the characteristics of the molecule or sequence. In different embodiments, the function of the functional fragment or functional variant may be reduced but still significantly present, e.g., binding of the functional variant may be at least 50%, at least 60%, at least 70%, at least 80%, or at least 90% of the parent molecule or sequence. However, in other embodiments, binding of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the sequences suitable for use herein may be altered such that they vary in sequence from the naturally occurring or native sequences from which they were derived, while retaining the desirable activity of the native sequences.

For example, the amino acid sequences of the VH, VL, CH1, and CL domains of the polypeptide chains of the binding agent of the invention may be derived from amino acid sequences of VH, VL, CH1, and CL domains of immunoglobulins but may be altered compared to the domains from which they are derived. For example, according to the invention, a VH or VL derived from an immunoglobulin comprises an amino acid sequence that can be identical to the amino acid sequence of the respective VH or VL it is derived from, or it can differ in one or more amino acid positions compared to the sequence of the respective parent VH or VL. For example, a VH domain of a binding agent of the invention may comprise an amino acid sequence comprising one or more amino acid insertions, amino acid additions, amino acid deletions and/or amino acid substitutions compared to the amino acid sequence of the VH domain it is derived from. For example, a VL domain of a binding agent of the invention may comprise an amino acid sequence comprising one or more amino acid insertions, amino acid additions, amino acid deletions and/or amino acid substitutions compared to the amino acid sequence of the VL domain it is derived from. Preferably, a VH or VL having an amino acid sequence that is a functional variant of the amino acid sequence of the parent VH or VL provides the same or essentially the same functions as the amino acid sequence of the parent VH or VL, e.g., in terms of binding specificity, binding strength etc. However, as one of ordinary skill in the art will be aware, in some embodiments, it may also be preferable to provide a functional variant of an amino acid sequence, e.g., of a VH or VL, which has altered characteristics compared to the amino acid sequence of the parent molecule. The same considerations apply to amino acid sequences of, e.g., CDRs, and to other amino acid sequences, e.g., those of CH1, and/or CL domains. In some embodiments, variants of the CH1 and CL sequences described herein have the ability to interact, e.g., the ability to bind to each other. As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present invention means “made through genetic engineering”. Preferably, a “recombinant object” such as a recombinant nucleic acid in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

“Physiological pH” as used herein refers to a pH of about 7.35 to about 7.45, with the average at about 7.40.

The term “genetic modification” or simply “modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding a binding agent is transiently transfected into cells. RNA can be transfected into cells to transiently express its coded protein.

### Claudin 6 (CLDN6)

Claudins are a family of proteins that are the most important components of tight junctions, where they establish the paracellular barrier that controls the flow of molecules in the intercellular space between cells of an epithelium. Claudins are transmembrane proteins spanning the membrane 4 times with the N-terminal and the C-terminal end both located in the cytoplasm. The first extracellular loop, termed EL1 or ECL1, consists on average of 53 amino acids, and the second extracellular loop, termed EL2 or ECL2, consists of around 24 amino acids. The term “claudin 6” or “CLDN6” preferably relates to human CLDN6, and, in particular, to a protein comprising, preferably consisting of the amino acid sequence of SEQ ID NO: 1 or SEQ ID NO: 2 of the sequence listing or a variant of said amino acid sequence. The first extracellular loop of CLDN6 preferably comprises amino acids 28 to 80 or 29 to 81, more preferably amino acids 28 to 76 of the amino acid sequence shown in SEQ ID NO: 1 or the amino acid sequence shown in SEQ ID NO: 2. The second extracellular loop of CLDN6 preferably comprises amino acids 138 to 160, preferably amino acids 141 to 159, more preferably amino acids 145 to 157 of the amino acid sequence shown in SEQ ID NO: 1 or the amino acid sequence shown in SEQ ID NO: 2. Said first and second extracellular loops preferably form the extracellular portion of CLDN6.

CLDN6 is expressed in tumors of various origins, with the only adult normal tissue expressing CLDN6 being placenta.

CLDN6 has been found to be expressed, for example, in ovarian cancer, lung cancer, testicular cancer, endometrial cancer, gastric cancer, breast cancer, hepatic cancer, pancreatic cancer, skin cancer, melanomas, head neck cancer, sarcomas, bile duct cancer, renal cell cancer, and urinary bladder cancer. CLDN6 is a particularly preferred target for the prevention and/or treatment of ovarian cancer, in particular ovarian adenocarcinoma and ovarian teratocarcinoma, fallopian tube cancer peritoneal cancer, lung cancer, including small cell lung cancer (SCLC) and non-small cell lung cancer (NSCLC), in particular squamous cell lung carcinoma and adenocarcinoma or non-small cell lung cancer (NSCLC) of non-squamous type, gastric cancer, breast cancer, hepatic cancer, pancreatic cancer, skin cancer, in particular basal cell carcinoma and squamous cell carcinoma, malignant melanoma, head and neck cancer, in particular malignant pleomorphic adenoma, sarcoma, in particular synovial sarcoma and carcinosarcoma, bile duct cancer, cancer of the urinary bladder, in particular transitional cell carcinoma and papillary carcinoma, kidney cancer, in particular renal cell carcinoma including clear cell renal cell carcinoma and papillary renal cell carcinoma, colon cancer, small bowel cancer, including cancer of the ileum, in particular small bowel adenocarcinoma and adenocarcinoma of the ileum, testicular embryonal carcinoma, placental choriocarcinoma, cervical cancer, testicular cancer, in particular testicular seminoma, testicular teratoma and embryonic testicular cancer, uterine cancer, germ cell tumors such as a teratocarcinoma or an embryonal carcinoma, in particular germ cell tumors of the testis, and the metastatic forms thereof. In some embodiments, the cancer disease associated with CLDN6 expression is selected from the group consisting of ovarian cancer, lung cancer, metastatic ovarian cancer and metastatic lung cancer. Preferably, the ovarian cancer is a carcinoma or an adenocarcinoma. Preferably, the lung cancer is a carcinoma or an adenocarcinoma, and preferably is bronchiolar cancer such as a bronchiolar carcinoma or bronchiolar adenocarcinoma. As used herein, the term “CLDN6-positive cancer” relates to a cancer involving cancer cells expressing CLDN6, preferably on the surface of said cancer cells.

According to the invention, CLDN6 is not substantially expressed in a cell if the level of expression is lower compared to expression in placenta cells or placenta tissue. Preferably, the level of expression is less than 10%, preferably less than 5%, 3%, 2%, 1%, 0.5%, 0.1% or 0.05% of the expression in placenta cells or placenta tissue or even lower. Preferably, CLDN6 is not substantially expressed in a cell if the level of expression exceeds the level of expression in non-cancerous tissue other than placenta by no more than 2-fold, preferably 1.5-fold, and preferably does not exceed the level of expression in said non-cancerous tissue. Preferably, CLDN6 is not substantially expressed in a cell if the level of expression is below the detection limit and/or if the level of expression is too low to allow binding by CLDN6-specific antibodies added to the cell.

According to the invention, CLDN6 is expressed in a cell if the level of expression exceeds the level of expression in non-cancerous tissue other than placenta preferably by more than 2-fold, preferably 10-fold, 100-fold, 1,000-fold, or 10,000-fold. Preferably, CLDN6 is expressed in a cell if the level of expression is above the detection limit and/or if the level of expression is high enough to allow binding by CLDN6-specific antibodies added to the cell. Preferably, CLDN6 expressed in a cell is expressed or exposed on the surface of said cell.

### Cluster of Differentiation 3 (CD3)

The second target molecule of the binding agents described herein is CD3 (cluster of differentiation 3).

The CD3 complex is a T cell-specific antigen. A T cell-specific antigen is an antigen on the surface of T cells.

The CD3 complex denotes an antigen that is expressed on mature human T-cells, thymocytes and a subset of natural killer cells as part of the multimolecular T-cell receptor (TCR) complex. The T-cell co-receptor is a protein complex and is composed of four distinct chains. In mammals, the complex contains a CD3γ chain, a CD3δ chain, and two CD3ε chains. These chains associate with a molecule known as the T-cell receptor (TCR) and the ζ-chain to generate an activation signal in T lymphocytes. The TCR, ζ-chain, and CD3 molecules together comprise the TCR complex. The human CD3 epsilon is indicated in GenBank Accession No. NM_000733 and comprises SEQ ID NO: 3. The human CD3 gamma is indicated in GenBank Accession No. NM_000073. The human CD3 delta is indicated in GenBank Accession No. NM_000732. CD3 is responsible for the signal transduction of the TCR. As described by Lin and Weiss, Journal of Cell Science 114, 243-244 (2001), activation of the TCR complex by binding of MHC-presented specific antigen epitopes results in the phosphorylation of immunoreceptor tyrosine-based activation motifs (ITAMs) by Src family kinases, triggering recruitment of further kinases which results in T-cell activation including Ca²⁺ release. Clustering of CD3 on T cells, e.g., by immobilized anti-CD3-antibodies, leads to T-cell activation similar to the engagement of the T-cell receptor, but independent from its clone typical specificity.

As used herein, “CD3” includes human CD3 and denotes an antigen that is expressed on human T cells as part of the multimolecular T-cell receptor complex.

In some embodiments, the binding agent described herein recognizes the epsilon-chain of CD3, particular, it recognizes an epitope that corresponds to the first 27 N-terminal amino acids of CD3 epsilon or functional fragments of this 27 amino acid stretch.

### Binding Agents

The present disclosure describes binding agents such as bispecific, trivalent binding agents capable of binding at least to an epitope of CD3 and an epitope of CLDN6. The binding agent comprises at least three binding domains, wherein the first binding domain is capable of binding to CD3 and the second and third binding domains are capable of binding to CLDN6, and wherein the second and third binding domains bind to the same or different epitopes of CLDN6. In some embodiments, the second and third binding domains of the binding agents described herein bind to the same epitope of CLDN6. In some embodiments, the sequences of the second and third binding domains are identical or essentially identical.

In some embodiments, the binding agents described herein are recombinant molecules.

The term “epitope” refers to a part or fragment of a molecule or antigen such as CD3 and/or CLDN6 that is recognized by a binding agent. For example, the epitope may be recognized by an antibody or any other binding protein. An epitope may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, an epitope is between about 10 and about 25 amino acids in length. The term “epitope” includes structural epitopes.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (abbreviated herein as C_{H} or CH). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as V_{L} or VL) and a light chain constant region (abbreviated herein as C_{L} or CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, reference to amino acid positions in the constant regions in the present invention is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242). In general, CDRs described herein are Kabat defined. In some embodiments, an immunoglobulin is an antibody.

Throughout this document, a reference to a heavy chain (HC) or a light chain (LC) does not necessarily imply the presence of an entire heavy chain (HC) or a light chain (LC) but is used as shorthand to indicate the presence of at least a relevant or distinguishing portion of a heavy chain (HC) or a light chain (LC). For example, if a (Fab)-(scFv)2-based bispecific antibody has two chains and one comprises a variable region of a heavy chain (VH) derived from a parental immunoglobulin as well as a scFv, and the other chain comprises a variable region of a light chain (VL) derived from an parental immunoglobulin as well as a scFv, the two chains may respectively be referred to as the heavy chain (HC) and the light chain (LC). This can be the case even though neither of the chains in fact comprises a heavy or light chain, and both chains comprise a scFv, meaning that they both comprise elements derived from a parental heavy and a parental light chain.

The term “antibody” (Ab) in the context of the present invention refers to an immunoglobulin molecule, a fragment of an immunoglobulin molecule, or a derivative of either thereof, which has the ability to bind, preferably specifically bind to an antigen. In some embodiments, binding takes place under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen). The variable regions of the heavy and light chains of the immunoglobulin molecule contain a binding domain that interacts with an antigen. The term “antigen-binding region”, “binding region” or “binding domain”, as used herein, refers to the region or domain which interacts with the antigen and typically comprises both a VH region and a VL region. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody as used herein, unless otherwise stated or clearly contradicted by context, includes fragments of an antibody that are antigen-binding fragments, i.e., retain the ability to specifically bind to the antigen, and antibody derivatives, i.e., constructs that are derived from an antibody. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 November; 21(11):484-90); (vi) camelid or Nanobody molecules (Revets et al; Expert Opin Biol Ther. 2005 January; 5(1):111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

The phrase “single chain Fv” or “scFv” refers to an antibody in which the variable domains of the heavy chain and of the light chain (VH and VL) of a traditional two chain antibody have been joined to form one chain. Optionally, a linker (usually a peptide) is inserted between the two chains to allow for proper folding and creation of an active binding site.

An antibody can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally-occurring IgG1 antibody, including variations in the constant regions.

In various embodiments, an antibody is an IgG1 antibody, more particularly an IgG1, kappa or IgG1, lambda isotype (i.e. IgG1, κ, λ), an IgG2a antibody (e.g. IgG2a, κ, λ), an IgG2b antibody (e.g. IgG2b, κ, λ), an IgG3 antibody (e.g. IgG3, κ, λ) or an IgG4 antibody (e.g. IgG4, κ, λ).

The term “monoclonal antibody” as used herein refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity. The terms “variable region” or “variable domain” as used in the context of chimeric antibodies, refer to a region which comprises the CDRs and framework regions of both the heavy and light chains of the immunoglobulin. Chimeric antibodies may be generated by using standard DNA techniques as described in Sambrook et al., 1989, Molecular Cloning: A laboratory Manual, New York: Cold Spring Harbor Laboratory Press, Ch. 15. The chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties. The term “human antibody” as used herein, refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse or rat, have been grafted onto human framework sequences. Human monoclonal antibodies can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known. Human monoclonal antibodies can thus e.g. be generated using transgenic or transchromosomal mice or rats carrying parts of the human immune system rather than the mouse or rat system. Accordingly, in some embodiments, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogeneous animal antibody machinery, see e.g. Mendez et al. 1997 Nat Genet. 15(2):146-56. The term “full-length” when used in the context of an antibody indicates that the antibody is not a fragment, but contains all of the domains of the particular isotype normally found for that isotype in nature, e.g., the VH, CH1, CH2, CH3, hinge, VL and CL domains for an IgG1 antibody. When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain. As used herein, the term “binding” or “capable of binding” in the context of the binding of a binding agent, e.g., an antibody, to a predetermined antigen or epitope typically refers to a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, for instance, when determined using Bio-Layer Interferometry (BLI), when determined using surface plasmon resonance (SPR) technology in a BIAcore 3000 instrument using the antigen as the ligand and the binding agent as the analyte or, when determined using a quartz crystal microbalance system using target (CLDN6)-expressing cells as “ligand”. In some embodiments, the binding agent binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen. The amount with which the affinity is lower is dependent on the K_{D} of the binding agent, so that when the K_{D} of the binding agent is very low (that is, the binding agent is highly specific), then the degree to which the affinity for the antigen is lower than the affinity for a non-specific antigen may be at least 10,000-fold.

The term “k_{d}” (sec⁻¹), as used herein, refers to the dissociation rate constant of a particular binding agent-antigen interaction. Said value is also referred to as the k_{off} value.

The term “K_{D}” (M), as used herein, refers to the dissociation equilibrium constant of a particular binding agent-antigen interaction.

The present invention also envisions binding agents comprising functional variants of the VL regions, VH regions, or one or more CDRs described herein. A functional variant of a VL, VH, or CDR used in the context of a binding agent still allows the binding agent to retain at least a substantial proportion (at least about 50%, 60%, 70%, 80%, 90%, 95% or more) of the affinity and/or the specificity/selectivity of the “reference” or “parent” binding agent and in some cases, such a binding agent may be associated with greater affinity, selectivity and/or specificity than the parent binding agent.

Such functional variants typically retain significant sequence identity to the parent sequence.

Exemplary variants include those which differ from VH and/or VL and/or CDR regions of the parent sequences mainly by conservative substitutions; for instance, up to 10, such as 9, 8, 7, 6, 5, 4, 3, 2 or 1 of the substitutions in the variant are conservative amino acid residue replacements.

Functional variants of sequences described herein such as VL regions, or VH regions, or sequences having a certain degree of homology or identity to sequences described herein such as VL regions, or VH regions preferably comprise modifications or variations in the non-CDR sequences, while the CDR sequences preferably remain unchanged.

A binding agent comprising variants of heavy and/or light chain variable regions sequences as described herein, e.g., comprising modifications in the CDRs and/or a certain degree of identity as described herein, may compete for binding to an antigen, e.g., CD3 and/or CLDN6, with another binding agent, e.g., a binding agent comprising heavy and light chain variable regions as described herein, or may have the specificity for an antigen of another binding agent, e.g., a binding agent comprising heavy and light chain variable regions as described herein.

The term “specificity” as used herein is intended to have the following meaning unless contradicted by context. Two binding agents have the “same specificity” if they bind to the same antigen and the same epitope.

The term “competes” and “competition” may refer to the competition between a first binding agent and a second binding agent to the same antigen. It is well known to a person skilled in the art how to test for competition of binding agents such as antibodies for binding to a target antigen. An example of such a method is a so-called cross-competition assay, which may e.g. be performed as an ELISA or by flow-cytometry. Alternatively, competition may be determined using biolayer interferometry.

Binding agents which compete for binding to a target antigen may bind different epitopes on the antigen, wherein the epitopes are so close to each other that a first binding agent binding to one epitope prevents binding of a second binding agent to the other epitope. In other situations, however, two different binding agents may bind the same epitope on the antigen and would compete for binding in a competition binding assay. Such binding agents binding to the same epitope are considered to have the same specificity herein. Thus, in some embodiments, binding agents binding to the same epitope are considered to bind to the same amino acids on the target molecule. That binding agents bind to the same epitope on a target antigen may be determined by standard alanine scanning experiments or antibody-antigen crystallization experiments known to a person skilled in the art. Preferably, binding agents or binding domains binding to different epitopes are not competing with each other for binding to their respective epitopes.

As described above, various formats of antibodies have been described in the art. The binding agent of the invention can in principle comprise sequences of an antibody of any isotype. Exemplary isotypes are IgG1, IgG2, IgG3, and IgG4. Either of the human light chain constant regions, kappa or lambda, may be used. In some embodiments, the sequences of a binding agent described herein such as CH1 and CL are derived from an antibody of the IgG1 isotype, for instance an IgG1,κ antibody.

Preferably, each of the antigen-binding regions or domains comprises a heavy chain variable region (VH) and a light chain variable region (VL), and wherein said variable regions each comprise three CDR sequences, CDR1, CDR2 and CDR3, respectively, and four framework sequences, FR1, FR2, FR3 and FR4, respectively. Furthermore, preferably, the binding agent described herein comprises a heavy chain constant regions (CH), and a light chain constant regions (CL).

The term “binding agent” in the context of the present invention refers to any agent capable of binding to one or more desired antigens, e.g., CD3 and CLDN6. The term “binding agent” includes antibodies, antibody fragments, or any other binding protein, or any combination thereof. In some embodiments, the binding protein comprises antibody fragments such as Fab and scFv. Naturally occurring antibodies are generally monospecific, i.e. they bind to a single antigen. The present invention provides binding agents binding to a cytotoxic cell such as a T cell (by engaging the CD3 receptor) and a target cell such as a cancer cell (by engaging CLDN6). Such binding agents are at least bispecific or multispecific such as trispecific, tetraspecific and so on. In some embodiments, a binding agent described herein is be an artificial protein that is composed of fragments of two different antibodies (said fragments of two different antibodies forming three binding domains).

According to the invention, a bispecific binding agent, in particular a bispecific protein, is a molecule that has two different binding specificities and thus may bind to two epitopes. Particularly, the term “bispecific binding agent” as used herein includes an antibody-derived molecule comprising three antigen-binding sites, a first binding site having affinity for a first epitope and a second and third binding site having binding affinity for a second epitope distinct from the first.

The term “bispecific” in the context of the present invention refers to an agent comprising two different antigen-binding regions binding to different epitopes, in particular different epitopes on different antigens, e.g. CD3 and CLDN6.

“Multispecific binding agents” are molecules which have more than two different binding specificities.

In some embodiments, a binding agent described herein binding to CD3 and CLDN6 is at least trivalent. As used herein, “valent”, “valence”, “valencies”, or other grammatical variations thereof, mean the number of antigen binding sites or binding domains in a binding agent. In some embodiments, a binding agent described herein has at least one antigen binding site or binding domain for CD3 and at least two antigen binding sites or binding domains for CLDN6. Antigen binding sites binding to the same antigen may recognize the same epitope or different epitopes. In some embodiments, the binding agent described herein is in the format of a Fab-scFv₂ construct, i.e., a Fab fragment specific for CD3 is provided with two scFv fragments specific for CLDN6 at the C-terminus of the constant regions of the Fab fragment. In some embodiments, the binding agent is a dimer composed of two polypeptide chains preferably bound together by a disulfide bridge, in which the first polypeptide comprises an scFv linked to an additional VH domain through a CH1 polypeptide chain, and the second polypeptide comprises an scFv linked to an additional VL domain through a CL polypeptide chain. The disulfide bridge is preferably formed between a Cys residue in the CH1 and a Cys residue in the CL, such that the additional VH of the first polypeptide associates with the additional VL of the second polypeptide in an antigen-binding configuration, such that the binding agent as a whole includes three antigen-binding domains. Thus, in some embodiments, the binding agent comprises the heavy chain (Fd fragment) and light chain (L) of a Fab fragment which are able to heterodimerize and upon which scFv binding domains are incorporated (preferably at the C-terminus of Fd/L). In some embodiments, the VH and VL domains in the scFv moieties are connected by peptide linkers and/or the Fab chains and the scFv are connected by peptide linkers. In some embodiments, the VH and VL domains in the scFv moieties are connected by peptide linkers comprising the amino acid sequence (G₄S)ₓ, wherein x is 2, 3, 4, 5 or 6. In some embodiments, the Fab chains and the scFv are connected by a peptide linker comprising the amino acid sequence SGPG₃RS(G₄S)₂ or DVPG₂S. In some embodiments, a linker comprising the amino acid sequence SGPG₃RS(G₄S)₂ connects a scFv binding domain to a Fd fragment and a linker comprising the amino acid sequence DVPG₂S connects a scFv binding domain to an L fragment. In some embodiments, the scFv moieties bind to CLDN6 and the Fab moiety binds to CD3.

The term “linker” refers to any means that serves to join two distinct functional units (e.g. antigen binding moieties). Types of linkers include, but are not limited to, chemical linkers and polypeptide linkers. The sequences of the polypeptide linkers are not limited. In some embodiments, polypeptide linkers are preferably non-immunogenic and flexible, such as those comprising serine and glycine sequences. Depending on the particular construct, the linkers may be long or short.

In some embodiments, a linker connecting the VH and VL domains to form VH-VL or VL-VH scFv domains preferably comprises a flexible peptide linker such as a glycine-serine peptide linker. In some embodiments, the linker comprises the amino acid sequence (G₄S)ₓ, wherein x is 2, 3, 4, 5 or 6. In some embodiments, in case of a scFv domain comprising the VH and VL domains in the VH-VL orientation the linker comprises the amino acid sequence (G₄S)₄. In some embodiments, in case of a scFv domain comprising the VH and VL domains in the VL-VH orientation the linker comprises the amino acid sequence (G₄S)₅.

In some embodiments, a linker connecting a scFv domain and a Fd domain, preferably at the C-terminus of CH1, comprises the amino acid sequence DVPG₂S or SGPG₃RS(G₄S)₂, preferably SGPG₃RS(G₄S)₂. In some embodiments, a linker connecting a scFv domain and a L domain, preferably at the C-terminus of CL, preferably comprises the amino acid sequence DVPG₂S or SGPG₃RS(G₄S)₂, preferably DVPG₂S.

Binding agents may also comprise an amino acid sequence for facilitating secretion of the molecule, such as a N-terminal secretion signal, and/or one or more epitope tags facilitating binding, purification or detection of the molecule.

According to some embodiments, each of the polypeptide chains of a binding agent described herein comprises a signal peptide.

Such signal peptides are sequences, which typically exhibit a length of about 15 to 30 amino acids and are preferably located at the N-terminus of a polypeptide chain, without being limited thereto. Signal peptides as defined herein preferably allow the transport of the polypeptide chain(s), e.g., as encoded by RNA, into a defined cellular compartment, preferably the cell surface, the endoplasmic reticulum (ER) or the endosomal-lysosomal compartment.

The signal peptide sequence as defined herein includes, without being limited thereto, the signal peptide sequence of an immunoglobulin, e.g., the signal peptide sequence of an immunoglobulin heavy chain variable region or the signal peptide sequence of an immunoglobulin light chain variable region, wherein the immunoglobulin may be human immunoglobulin. In some embodiments, the signal peptide sequence is the signal peptide sequence of an MHC molecule, e.g., MHC class I molecule, wherein the MHC molecule may be a human MHC molecule (HLA molecule).

In some embodiments, the secretion signal is a signal sequence (e.g., an amino acid sequence comprising amino acids 1 to 26 of SEQ ID NO: 4) that allows a sufficient passage through the secretory pathway and/or secretion of the binding agent or the polypeptide chains thereof into the extracellular environment. In some embodiments, the secretion signal sequence is cleavable and is removed from the mature binding agent. In some embodiments, the secretion signal sequence is chosen with respect to the cell or organism wherein the binding agent is produced in.

In a further embodiment, the binding agents described herein are linked or conjugated to one or more therapeutic moieties, such as a cytokine, an immune-suppressant, and/or an immune-stimulatory molecule.

In some embodiments, the binding agent described herein comprises a Fab antibody fragment comprising the first binding domain. In some embodiments, the binding agent described herein comprises two scFv antibody fragments comprising the second and third binding domains which are covalently linked to the Fab antibody fragment comprising the first binding domain. In some embodiments, the binding agent comprises the scFv antibody fragments covalently linked to the C-terminus of each chain of the Fab antibody fragment.

The CH1 and CL sequences of a binding agent described herein may each be of any isotype, including, but not limited to, IgG1, IgG2, IgG3 and IgG4, and may comprise one or more mutations or modifications. In some embodiments, each of the CH1 and CL sequences is of the IgG1 isotype or derived therefrom, optionally with one or more mutations or modifications.

In some embodiments of the invention, a binding agent described herein does not comprise a full-length antibody. In some embodiments of the invention, a binding agent described herein does not comprise CH2 and CH3 domains of an antibody. In some embodiments of the invention, a binding agent described herein does not comprise a Fc region. In some embodiments of the invention, a binding agent described herein does not comprise Fc sequences which are able of exerting effector-functions.

The term “effector functions” in the context of the present invention includes any functions mediated by components of the immune system that result, for example, in the killing of diseased cells such as tumor cells, or in the inhibition of tumor growth and/or inhibition of tumor development, including inhibition of tumor dissemination and metastasis. Preferably, the effector functions in the context of the present invention are T cell mediated effector functions. Such functions comprise ADCC, ADCP or CDC.

Antibody-dependent cell-mediated cytotoxicity (ADCC) is the killing of an antibody-coated target cell by a cytotoxic effector cell through a nonphagocytic process, characterised by the release of the content of cytotoxic granules or by the expression of cell death-inducing molecules. ADCC is independent of the immune complement system that also lyses targets but does not require any other cell. ADCC is triggered through interaction of target-bound antibodies (belonging to IgG or IgA or IgE classes) with certain Fc receptors (FcRs), glycoproteins present on the effector cell surface that bind the Fc region of immunoglobulins (Ig). Effector cells that mediate ADCC include natural killer (NK) cells, monocytes, macrophages, neutrophils, eosinophils and dendritic cells. ADCC is a rapid effector mechanism whose efficacy is dependent on a number of parameters (density and stability of the antigen on the surface of the target cell; antibody affinity and FcR-binding affinity). ADCC involving human IgG1, the most used IgG subclass for therapeutic antibodies, is highly dependent on the glycosylation profile of its Fc portion and on the polymorphism of Fcγ receptors.

Antibody-dependent cellular phagocytosis (ADCP) is one crucial mechanism of action of many antibody therapies. It is defined as a highly regulated process by which antibodies eliminate bound targets via connecting its Fc domain to specific receptors on phagocytic cells, and eliciting phagocytosis. Unlike ADCC, ADCP can be mediated by monocytes, macrophages, neutrophils, and dendritic cells, through FcγRIIa, FcγRI, and FcγRIIIa, of which FcγRIIa (CD32a) on macrophages represent the predominant pathway.

Complement-dependent cytotoxicity (CDC) is another cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the C_{H}2 domains of participating antibody molecules such as IgG molecules (C1q is one of three subcomponents of complement C1). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. Preferably, the complement cascade ends in the formation of a membrane attack complex, which creates pores in the cell membrane that facilitate free passage of water and solutes into and out of the cell.

In some embodiments, the binding agent comprises two polypeptide chains forming a binding domain with specificity for CD3 and two binding domains with specificity for CLDN6. In some embodiments, the two polypeptide chains are enoded by two RNA molecules. In some embodiments, the binding agent is a dimer composed of two polypeptide chains, in which the first polypeptide comprises a scFv which is specific for CLDN6 linked to an additional VH domain through a constant region 1 of a heavy chain of an immunoglobulin (CH1) and the second polypeptide comprises a scFv which is specific for CLDN6 linked to an additional VL domain through a constant region of a light chain of an immunoglobulin (CL). In some embodiments, the two polypeptide chains are bound together by a disulfide bridge. The disulfide bridge is preferably formed between a Cys residue in the CH1 domain and a Cys residue in the CL domain, such that the additional VH domain of the first polypeptide associates with the additional VL domain of the second polypeptide in a CD3-binding configuration, such that the binding agent as a whole includes three antigen-binding domains. In some embodiments, the binding domain which is specific for CD3 is comprised by a Fab fragment and the binding domains which are specific for CLDN6 are each comprised by a scFv. In some embodiments, each chain of the Fab fragment is linked to one scFv and the scFvs are preferably linked at the C-termini of the Fab fragment. According to the invention, the VH and VL domains in the scFv moieties are preferably connected by peptide linkers such as a peptide linker comprising the amino acid sequence (G₄S)₄, and the Fab chains and the scFv are preferably connected by peptide linkers such as a peptide linker comprising the amino acid sequence SGPG₃RS(G₄S)₂ or DVPG₂S.

In some embodiments, the binding agent comprises (i) a first polypeptide chain comprising a variable region of a heavy chain (VH) derived from an immunoglobulin with specificity for CD3 (VH(CD3)), a VH derived from an immunoglobulin with specificity for CLDN6 (VH(CLDN6)) and a variable region of a light chain (VL) derived from an immunoglobulin with specificity for CLDN6 (VL(CLDN6)); and (ii) a second polypeptide chain comprising a variable region of a light chain (VL) derived from an immunoglobulin with specificity for CD3 (VL(CD3)), a VH derived from an immunoglobulin with specificity for CLDN6 (VH(CLDN6)) and a variable region of a light chain (VL) derived from an immunoglobulin with specificity for CLDN6 (VL(CLDN6)). In some embodiments, the first polypeptide chain interacts with the second polypeptide chain to form the binding agent. In some embodiments, the VH(CD3) of the first polypeptide chain and the VL(CD3) of the second polypeptide chain interact to form a binding domain with specificity for CD3. In some embodiments, the VH(CLDN6) and the VL(CLDN6) of the first polypeptide chain interact to form a binding domain with specificity for CLDN6. In some embodiments, the VH(CLDN6) and the VL(CLDN6) of the second polypeptide chain interact to form a binding domain with specificity for CLDN6. In some embodiments, the first and the second polypeptide chains comprise a constant region 1 of a heavy chain (CH1) derived from an immunoglobulin or a functional variant thereof and a constant region of a light chain (CL) derived from an immunoglobulin or a functional variant thereof. In some embodiments, the immunoglobulin is IgG1. In some embodiments, the IgG1 is human IgG1. In some embodiments, the VH, the VL, and the CH1 on the first polypeptide chain are arranged, from N-terminus to C-terminus, in the order
VH(CD3)-CH1-VH(CLDN6)-VL(CLDN6), or
VH(CD3)-CH1-VL(CLDN6)-VH(CLDN6).

In some embodiments, the CH1 is connected to the VH(CLDN6) or VL(CLDN6) by a peptide linker. In some embodiments, the peptide linker comprises the amino acid sequence SGPGGGRS(G₄S)₂ or a functional variant thereof.

In some embodiments, the VH, the VL, and the CL on the second polypeptide chain are arranged, from N-terminus to C-terminus, in the order
VL(CD3)-CL-VH(CLDN6)-VL(CLDN6), or
VL(CD3)-CL-VL(CLDN6)-VH(CLDN6).

In some embodiments, the CL is connected to the VH(CLDN6) or VL(CLDN6) by a peptide linker. In some embodiments, the peptide linker comprises the amino acid sequence DVPGGS or a functional variant thereof.

In some embodiments, the VH(CLDN6) and the VL(CLDN6) are connected to one another by a peptide linker. In some embodiments, the peptide linker comprises the amino acid sequence (G₄S)ₓ or a functional variant thereof, wherein x is 2, 3, 4, 5 or 6. In some embodiments, the peptide linker comprises the amino acid sequence (G₄S)₄ or a functional variant thereof.

In some embodiments, the CH1 on the first polypeptide chain interacts with the CL on the second polypeptide chain.

In some embodiments, the CH1 comprises the amino acid sequence of amino acids 146 to 248 of SEQ ID NO: 4 or a functional variant thereof.

In some embodiments, the CL comprises the amino acid sequence of amino acids 133 to 239 of SEQ ID NO: 6 or a functional variant thereof.

In some embodiments, the VH(CD3) comprises CDR1, CDR2 and CDR3 of the amino acid sequence of amino acids 27 to 145 of SEQ ID NO: 4 (respectively SEQ ID NO: 18, 19 and 20). In some embodiments, the VH(CD3) comprises a CDR1 comprising the amino acid sequence GYTFTRYT or a functional variant thereof, a CDR2 comprising the amino acid sequence INPSRGYT or a functional variant thereof, and a CDR3 comprising the amino acid sequence ARYYDDHYSLDY or ARYYDDHYCLDY or a functional variant thereof. In some embodiments, the VH(CD3) comprises the amino acid sequence of amino acids 27 to 145 of SEQ ID NO: 4 or a functional variant thereof. In some embodiments, the VL(CD3) comprises CDR1, CDR2 and CDR3 of the amino acid sequence of amino acids 27 to 132 of SEQ ID NO: 6 (respectively SEQ ID NO: 22, 23 and 24). In some embodiments, the VL(CD3) comprises a CDR1 comprising the amino acid sequence SSVSY or a functional variant thereof, a CDR2 comprising the amino acid sequence DTS or a functional variant thereof, and a CDR3 comprising the amino acid sequence QQWSSNPLT or a functional variant thereof. In some embodiments, the VL(CD3) comprises the amino acid sequence of amino acids 27 to 132 of SEQ ID NO: 6 or a functional variant thereof.

In some embodiments, the VH(CLDN6) comprises CDR1, CDR2 and CDR3 of the amino acid sequence of amino acids 267 to 383 of SEQ ID NO: 4 (respectively SEQ ID NO: 25, 26 and 27). In some embodiments, the VH(CLDN6) comprises a CDR1 comprising the amino acid sequence GYSFTGYT or a functional variant thereof, a CDR2 comprising the amino acid sequence INPYNGGT or a functional variant thereof, and a CDR3 comprising the amino acid sequence ARDYGFVLDY or a functional variant thereof. In some embodiments, the VH(CLDN6) comprises the amino acid sequence of amino acids 267 to 383 of SEQ ID NO: 4 or a functional variant thereof.

In some embodiments, the VL(CLDN6) comprises CDR1, CDR2 and CDR3 of the amino acid sequence of amino acids 404 to 510 of SEQ ID NO: 4 (respectively SEQ ID NO: 28, 29 and 30). In some embodiments, the VL(CLDN6) comprises a CDR1 comprising the amino acid sequence SSVSY or a functional variant thereof, a CDR2 comprising the amino acid sequence STS or a functional variant thereof, and a CDR3 comprising the amino acid sequence QQRSNYPPWT or a functional variant thereof. In some embodiments, the VL(CLDN6) comprises CDR1, CDR2 and CDR3 of the amino acid sequence of amino acids 404 to 510 of SEQ ID NO: 4 and a serine residue in position +15 relative to CDR1 (corresponds to position 449 of SEQ ID NO: 4). In some embodiments, the VL(CLDN6) comprises CDR1, CDR2 and CDR3 of the amino acid sequence of amino acids 404 to 510 of SEQ ID NO: 4 and a serine residue in position −3 relative to CDR2 (corresponds to position 449 of SEQ ID NO: 4). In some embodiments, the VL(CLDN6) comprises CDR1, CDR2 and CDR3 of the amino acid sequence of amino acids 404 to 510 of SEQ ID NO: 4, a sequence having at least 99%, 98%, 97%, 96%, 95%, 90%, 85%, or 80% identity to the amino acid sequence of amino acids 404 to 510 of SEQ ID NO: 4, and a serine residue in the position corresponding to position 449 of SEQ ID NO: 4. In some embodiments, the VL(CLDN6) comprises the amino acid sequence of amino acids 404 to 510 of SEQ ID NO: 4 or a functional variant thereof.

A serine residue in position +15 relative to CDR1 means that the 15th amino acid position after the end of the CDR1 is a serine residue. A serine residue in position −3 relative to CDR2 means that the third amino acid before the beginning of the CDR2 is a serine. These can for example respectively be represented by the following (N to C): XXXXX-Y₁₄-S and S-Y₂-ZZZ, wherein X represents a CDR1 amino acid, Y represents an intervening amino acid between CDRs, S represents a serine residue and Z represents a CDR2 amino acid.

In some embodiments, the VH(CD3) comprises CDR1, CDR2 and CDR3 of the amino acid sequence of amino acids 27 to 145 of SEQ ID NO: 4, the VL(CD3) comprises CDR1, CDR2 and CDR3 of the amino acid sequence of amino acids 27 to 132 of SEQ ID NO: 6, the VH(CLDN6) comprises CDR1, CDR2 and CDR3 of the amino acid sequence of amino acids 267 to 383 of SEQ ID NO: 4, and the VL(CLDN6) comprises CDR1, CDR2 and CDR3 of the amino acid sequence of amino acids 404 to 510 of SEQ ID NO: 4 and preferably the VL(CLDN6) comprises a serine residue in position +15 relative to CDR1 (corresponds to position 449 of SEQ ID NO: 4) and/or a serine residue in position −3 relative to CDR2 (corresponds to position 449 of SEQ ID NO: 4).

In some embodiments, the VH(CD3) comprises the amino acid sequence of amino acids 27 to 145 of SEQ ID NO: 4 or a functional variant thereof, the VL(CD3) comprises the amino acid sequence of amino acids 27 to 132 of SEQ ID NO: 6 or a functional variant thereof, the VH(CLDN6) comprises the amino acid sequence of amino acids 267 to 383 of SEQ ID NO: 4 or a functional variant thereof, and/or the VL(CLDN6) comprises the amino acid sequence of amino acids 404 to 510 of SEQ ID NO: 4 or a functional variant thereof.

In some embodiments, a first polypeptide chain comprises the amino acid sequence of amino acids 27 to 510 of SEQ ID NO: 4, an amino acid sequence having at least 99%, 98%, 97%, 96%, 95%, 90%, 85%, or 80% identity to the amino acid sequence of amino acids 27 to 510 of SEQ ID NO: 4, or a functional fragment of the amino acid sequence of amino acids 27 to 510 of SEQ ID NO: 4, or the amino acid sequence having at least 99%, 98%, 97%, 96%, 95%, 90%, 85%, or 80% identity to the amino acid sequence of amino acids 27 to 510 of SEQ ID NO: 4. In some embodiments, a first polypeptide chain comprises the amino acid sequence of amino acids 27 to 510 of SEQ ID NO: 4.

In these and other embodiments, RNA encoding a first polypeptide chain comprises the nucleotide sequence of nucleotides 132 to 1583 of SEQ ID NO: 5, a nucleotide sequence having at least 99%, 98%, 97%, 96%, 95%, 90%, 85%, or 80% identity to the nucleotide sequence of nucleotides 132 to 1583 of SEQ ID NO: 5, or a functional fragment of the nucleotide sequence of nucleotides 132 to 1583 of SEQ ID NO: 5, or the nucleotide sequence having at least 99%, 98%, 97%, 96%, 95%, 90%, 85%, or 80% identity to the nucleotide sequence of nucleotides 132 to 1583 of SEQ ID NO: 5. In some embodiments, RNA encoding a first polypeptide chain comprises the nucleotide sequence of nucleotides 132 to 1583 of SEQ ID NO: 5.

In some embodiments, a second polypeptide chain comprises the amino acid sequence of amino acids 27 to 489 of SEQ ID NO: 6, an amino acid sequence having at least 99%, 98%, 97%, 96%, 95%, 90%, 85%, or 80% identity to the amino acid sequence of amino acids 27 to 489 of SEQ ID NO: 6, or a functional fragment of the amino acid sequence of amino acids 27 to 489 of SEQ ID NO: 6, or the amino acid sequence having at least 99%, 98%, 97%, 96%, 95%, 90%, 85%, or 80% identity to the amino acid sequence of amino acids 27 to 489 of SEQ ID NO: 6. In some embodiments, a second polypeptide chain comprises the amino acid sequence of amino acids 27 to 489 of SEQ ID NO: 6.

In these and other embodiments, RNA encoding a second polypeptide chain comprises the nucleotide sequence of nucleotides 132 to 1520 of SEQ ID NO: 7, a nucleotide sequence having at least 99%, 98%, 97%, 96%, 95%, 90%, 85%, or 80% identity to the nucleotide sequence of nucleotides 132 to 1520 of SEQ ID NO: 7, or a functional fragment of the nucleotide sequence of nucleotides 132 to 1520 of SEQ ID NO: 7, or the nucleotide sequence having at least 99%, 98%, 97%, 96%, 95%, 90%, 85%, or 80% identity to the nucleotide sequence of nucleotides 132 to 1520 of SEQ ID NO: 7. In some embodiments, RNA encoding a second polypeptide chain comprises the nucleotide sequence of nucleotides 132 to 1520 of SEQ ID NO: 7.

In some embodiments, (i) a first polypeptide chain comprises the amino acid sequence of amino acids 27 to 510 of SEQ ID NO: 4, an amino acid sequence having at least 99%, 98%, 97%, 96%, 95%, 90%, 85%, or 80% identity to the amino acid sequence of amino acids 27 to 510 of SEQ ID NO: 4, or a functional fragment of the amino acid sequence of amino acids 27 to 510 of SEQ ID NO: 4, or the amino acid sequence having at least 99%, 98%, 97%, 96%, 95%, 90%, 85%, or 80% identity to the amino acid sequence of amino acids 27 to 510 of SEQ ID NO: 4, and (ii) a second polypeptide chain comprises the amino acid sequence of amino acids 27 to 489 of SEQ ID NO: 6, an amino acid sequence having at least 99%, 98%, 97%, 96%, 95%, 90%, 85%, or 80% identity to the amino acid sequence of amino acids 27 to 489 of SEQ ID NO: 6, or a functional fragment of the amino acid sequence of amino acids 27 to 489 of SEQ ID NO: 6, or the amino acid sequence having at least 99%, 98%, 97%, 96%, 95%, 90%, 85%, or 80% identity to the amino acid sequence of amino acids 27 to 489 of SEQ ID NO: 6. In some embodiments, a first polypeptide chain comprises the amino acid sequence of amino acids 27 to 510 of SEQ ID NO: 4 and a second polypeptide chain comprises the amino acid sequence of amino acids 27 to 489 of SEQ ID NO: 6. In these and other embodiments, (i) RNA encoding a first polypeptide chain comprises the nucleotide sequence of nucleotides 132 to 1583 of SEQ ID NO: 5, a nucleotide sequence having at least 99%, 98%, 97%, 96%, 95%, 90%, 85%, or 80% identity to the nucleotide sequence of nucleotides 132 to 1583 of SEQ ID NO: 5, or a functional fragment of the nucleotide sequence of nucleotides 132 to 1583 of SEQ ID NO: 5, or the nucleotide sequence having at least 99%, 98%, 97%, 96%, 95%, 90%, 85%, or 80% identity to the nucleotide sequence of nucleotides 132 to 1583 of SEQ ID NO: 5, and (ii) RNA encoding a second polypeptide chain comprises the nucleotide sequence of nucleotides 132 to 1520 of SEQ ID NO: 7, a nucleotide sequence having at least 99%, 98%, 97%, 96%, 95%, 90%, 85%, or 80% identity to the nucleotide sequence of nucleotides 132 to 1520 of SEQ ID NO: 7, or a functional fragment of the nucleotide sequence of nucleotides 132 to 1520 of SEQ ID NO: 7, or the nucleotide sequence having at least 99%, 98%, 97%, 96%, 95%, 90%, 85%, or 80% identity to the nucleotide sequence of nucleotides 132 to 1520 of SEQ ID NO: 7. In some embodiments, RNA encoding a first polypeptide chain comprises the nucleotide sequence of nucleotides 132 to 1583 of SEQ ID NO: 5 and RNA encoding a second polypeptide chain comprises the nucleotide sequence of nucleotides 132 to 1520 of SEQ ID NO: 7.

According to some embodiments, a signal peptide is fused, either directly or through a linker, to a polypeptide chain described herein. Accordingly, in some embodiments, a signal peptide is fused to the above described amino acid sequences.

In some embodiments, a signal sequence comprises the amino acid sequence of amino acids 1 to 26 of SEQ ID NO: 4, an amino acid sequence having at least 99%, 98%, 97%, 96%, 95%, 90%, 85%, or 80% identity to the amino acid sequence of amino acids 1 to 26 of SEQ ID NO: 4, or a functional fragment of the amino acid sequence of amino acids 1 to 26 of SEQ ID NO: 4, or the amino acid sequence having at least 99%, 98%, 97%, 96%, 95%, 90%, 85%, or 80% identity to the amino acid sequence of amino acids 1 to 26 of SEQ ID NO: 4. In some embodiments, a signal sequence comprises the amino acid sequence of amino acids 1 to 26 of SEQ ID NO: 4.

In these and other embodiments, RNA encoding a signal sequence (i) comprises the nucleotide sequence of nucleotides 54 to 131 of SEQ ID NO: 5, a nucleotide sequence having at least 99%, 98%, 97%, 96%, 95%, 90%, 85%, or 80% identity to the nucleotide sequence of nucleotides 54 to 131 of SEQ ID NO: 5, or a functional fragment of the nucleotide sequence of nucleotides 54 to 131 of SEQ ID NO: 5, or the nucleotide sequence having at least 99%, 98%, 97%, 96%, 95%, 90%, 85%, or 80% identity to the nucleotide sequence of nucleotides 54 to 131 of SEQ ID NO: 5. In some embodiments, RNA encoding a signal sequence comprises the nucleotide sequence of nucleotides 54 to 131 of SEQ ID NO: 5.

In some embodiments, a first polypeptide chain comprises the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a functional fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4. In some embodiments, a first polypeptide chain comprises the amino acid sequence of SEQ ID NO: 4.

In these and other embodiments, RNA encoding a first polypeptide chain comprises the nucleotide sequence of nucleotides 54 to 1583 of SEQ ID NO: 5, a nucleotide sequence having at least 99%, 98%, 97%, 96%, 95%, 90%, 85%, or 80% identity to the nucleotide sequence of nucleotides 54 to 1583 of SEQ ID NO: 5, or a functional fragment of the nucleotide sequence of nucleotides 54 to 1583 of SEQ ID NO: 5, or the nucleotide sequence having at least 99%, 98%, 97%, 96%, 95%, 90%, 85%, or 80% identity to the nucleotide sequence of nucleotides 54 to 1583 of SEQ ID NO: 5. In some embodiments, RNA encoding a first polypeptide chain comprises the nucleotide sequence of nucleotides 54 to 1583 of SEQ ID NO: 5.

In further embodiments, RNA encoding a first polypeptide chain comprises the nucleotide sequence of SEQ ID NO: 5, a nucleotide sequence having at least 99%, 98%, 97%, 96%, 95%, 90%, 85%, or 80% identity to the nucleotide sequence of SEQ ID NO: 5, or a functional fragment of the nucleotide sequence of SEQ ID NO: 5, or the nucleotide sequence having at least 99%, 98%, 97%, 96%, 95%, 90%, 85%, or 80% identity to the nucleotide sequence of SEQ ID NO: 5. In some embodiments, RNA encoding a first polypeptide chain comprises the nucleotide sequence of SEQ ID NO: 5.

In some embodiments, a second polypeptide chain comprises the amino acid sequence of SEQ ID NO: 6, an amino acid sequence having at least 99%, 98%, 97%, 96%, 95%, 90%, 85%, or 80% identity to the amino acid sequence of SEQ ID NO: 6, or a functional fragment of the amino acid sequence of SEQ ID NO: 6, or the amino acid sequence having at least 99%, 98%, 97%, 96%, 95%, 90%, 85%, or 80% identity to the amino acid sequence of SEQ ID NO: 6. In some embodiments, a second polypeptide chain comprises the amino acid sequence of SEQ ID NO: 6.

In these and other embodiments, RNA encoding a second polypeptide chain comprises the nucleotide sequence of nucleotides 54 to 1520 of SEQ ID NO: 7, a nucleotide sequence having at least 99%, 98%, 97%, 96%, 95%, 90%, 85%, or 80% identity to the nucleotide sequence of nucleotides 54 to 1520 of SEQ ID NO: 7, or a functional fragment of the nucleotide sequence of nucleotides 54 to 1520 of SEQ ID NO: 7, or the nucleotide sequence having at least 99%, 98%, 97%, 96%, 95%, 90%, 85%, or 80% identity to the nucleotide sequence of nucleotides 54 to 1520 of SEQ ID NO: 7. In some embodiments, RNA encoding a second polypeptide chain comprises the nucleotide sequence of nucleotides 54 to 1520 of SEQ ID NO: 7.

In further embodiments, RNA encoding a second polypeptide chain comprises the nucleotide sequence of SEQ ID NO: 7, a nucleotide sequence having at least 99%, 98%, 97%, 96%, 95%, 90%, 85%, or 80% identity to the nucleotide sequence of SEQ ID NO: 7, or a functional fragment of the nucleotide sequence of SEQ ID NO: 7, or the nucleotide sequence having at least 99%, 98%, 97%, 96%, 95%, 90%, 85%, or 80% identity to the nucleotide sequence of SEQ ID NO: 7. In some embodiments, RNA encoding a second polypeptide chain comprises the nucleotide sequence of SEQ ID NO: 7.

In some embodiments, (i) a first polypeptide chain comprises the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a functional fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4, and (ii) a second polypeptide chain comprises the amino acid sequence of SEQ ID NO: 6, an amino acid sequence having at least 99%, 98%, 97%, 96%, 95%, 90%, 85%, or 80% identity to the amino acid sequence of SEQ ID NO: 6, or a functional fragment of the amino acid sequence of SEQ ID NO: 6, or the amino acid sequence having at least 99%, 98%, 97%, 96%, 95%, 90%, 85%, or 80% identity to the amino acid sequence of SEQ ID NO: 6. In some embodiments, a first polypeptide chain comprises the amino acid sequence of SEQ ID NO: 4, and a second polypeptide chain comprises the amino acid sequence of SEQ ID NO: 6.

In these and other embodiments, (i) RNA encoding a first polypeptide chain comprises the nucleotide sequence of nucleotides 54 to 1583 of SEQ ID NO: 5, a nucleotide sequence having at least 99%, 98%, 97%, 96%, 95%, 90%, 85%, or 80% identity to the nucleotide sequence of nucleotides 54 to 1583 of SEQ ID NO: 5, or a functional fragment of the nucleotide sequence of nucleotides 54 to 1583 of SEQ ID NO: 5, or the nucleotide sequence having at least 99%, 98%, 97%, 96%, 95%, 90%, 85%, or 80% identity to the nucleotide sequence of nucleotides 54 to 1583 of SEQ ID NO: 5, and (ii) RNA encoding a second polypeptide chain comprises the nucleotide sequence of nucleotides 54 to 1520 of SEQ ID NO: 7, a nucleotide sequence having at least 99%, 98%, 97%, 96%, 95%, 90%, 85%, or 80% identity to the nucleotide sequence of nucleotides 54 to 1520 of SEQ ID NO: 7, or a functional fragment of the nucleotide sequence of nucleotides 54 to 1520 of SEQ ID NO: 7, or the nucleotide sequence having at least 99%, 98%, 97%, 96%, 95%, 90%, 85%, or 80% identity to the nucleotide sequence of nucleotides 54 to 1520 of SEQ ID NO: 7. In some embodiments, RNA encoding a first polypeptide chain comprises the nucleotide sequence of nucleotides 54 to 1583 of SEQ ID NO: 5, and RNA encoding a second polypeptide chain comprises the nucleotide sequence of nucleotides 54 to 1520 of SEQ ID NO: 7.

In further embodiments, (i) RNA encoding a first polypeptide chain comprises the nucleotide sequence of SEQ ID NO: 5, a nucleotide sequence having at least 99%, 98%, 97%, 96%, 95%, 90%, 85%, or 80% identity to the nucleotide sequence of SEQ ID NO: 5, or a functional fragment of the nucleotide sequence of SEQ ID NO: 5, or the nucleotide sequence having at least 99%, 98%, 97%, 96%, 95%, 90%, 85%, or 80% identity to the nucleotide sequence of SEQ ID NO: 5, and (ii) RNA encoding a second polypeptide chain comprises the nucleotide sequence of SEQ ID NO: 7, a nucleotide sequence having at least 99%, 98%, 97%, 96%, 95%, 90%, 85%, or 80% identity to the nucleotide sequence of SEQ ID NO: 7, or a functional fragment of the nucleotide sequence of SEQ ID NO: 7, or the nucleotide sequence having at least 99%, 98%, 97%, 96%, 95%, 90%, 85%, or 80% identity to the nucleotide sequence of SEQ ID NO: 7. In some embodiments, RNA encoding a first polypeptide chain comprises the nucleotide sequence of SEQ ID NO: 5, and RNA encoding a second polypeptide chain comprises the nucleotide sequence of SEQ ID NO: 7.

In some embodiments, the binding agent binds to an extracellular domain of CLDN6. In some embodiments, the binding agent binds to native epitopes of CLDN6 present on the surface of living cells. In some embodiments, the binding agent binds to the first extracellular loop of CLDN6. A binding agent described herein is specific for CLDN6, i.e., it has the ability of binding to CLDN6, i.e., the ability of binding to an epitope present in CLDN6, preferably an epitope located within the extracellular domains of CLDN6, in particular the first extracellular loop, preferably amino acid positions 28 to 76 or 29 to 81 of CLDN6 or the second extracellular loop, preferably amino acid positions 141 to 159 of CLDN6. In some embodiments, an agent having the ability of binding to CLDN6 binds to an epitope on CLDN6 which is not present on CLDN9. In some embodiments, an agent having the ability of binding to CLDN6 binds to an epitope on CLDN6 which is not present on CLDN4 and/or CLDN3. In some embodiments, an agent having the ability of binding to CLDN6 binds to an epitope on CLDN6 which is not present on a claudin protein other than CLDN6. In some embodiments, an agent having the ability of binding to CLDN6 preferably binds to CLDN6 but not to CLDN9 and preferably does not bind to CLDN4 and/or CLDN3. In some embodiments, an agent having the ability of binding to CLDN6 binds to CLDN6 expressed on the cell surface. In some embodiments, an agent having the ability of binding to CLDN6 binds to native epitopes of CLDN6 present on the surface of living cells.

The term “expressed on the cell surface” or “associated with the cell surface” means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein. “Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by e.g. antigen-specific antibodies added to the cells.

The term “extracellular portion” or “exodomain” in the context of the present invention refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

### Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In some embodiments of all aspects of the invention, the RNA encoding the binding agent, e.g., bispecific or multispecific binding agent, described herein is expressed in cells (e.g., liver cells) of the subject treated to provide the binding agent.

The nucleic acids described herein may be recombinant and/or isolated molecules.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA. In some embodiments of the present disclosure, the RNA is messenger RNA (mRNA) that relates to a RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In some embodiments, the mRNA is produced by in vitro transcription using a DNA template where DNA refers to a nucleic acid that contains deoxyribonucleotides.

In some embodiments of the present disclosure, the RNA is “replicon RNA” or simply a “replicon”, in particular “self-replicating RNA” or “self-amplifying RNA”. In one particularly preferred embodiment, the replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase. In some embodiments, the RNA described herein may have modified nucleosides. In some embodiments, the RNA comprises a modified nucleoside in place of at least one (e.g. every) uridine.

The term “uracil,” as used herein, describes one of the nucleobases that can occur in the nucleic acid of RNA. The structure of uracil is:

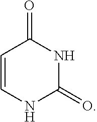

The term “uridine,” as used herein, describes one of the nucleosides that can occur in RNA. The structure of uridine is:

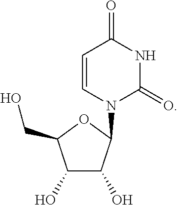 [SMILES: O=c1ccn([C@@H]2O[C@H](CO)[C@@H](O)[C@H]2O)c(=O)[nH]1]

UTP (uridine 5′-triphosphate) has the following structure:

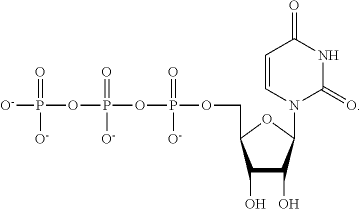

Pseudo-UTP (pseudouridine 5′-triphosphate) has the following structure:

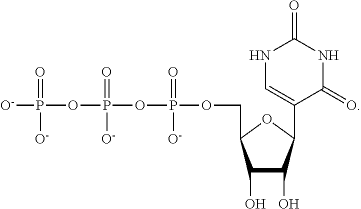

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m14P), which has the structure:

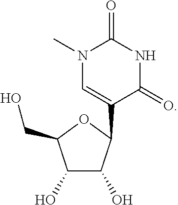 [SMILES: Cn1cc([C@@H]2O[C@H](CO)[C@@H](O)[C@H]2O)c(=O)[nH]c1=O]

N1-methyl-pseudo-UTP has the following structure:

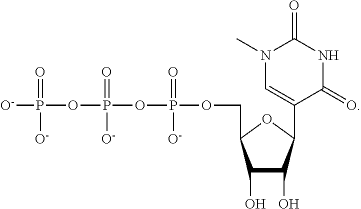

Another exemplary modified nucleoside is 5-methyl-uridine (m5U), which has the structure:

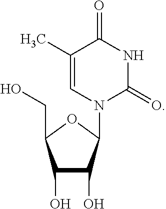

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (mli) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, the RNA comprises other modified nucleosides or comprises further modified nucleosides, e.g., modified cytidine. For example, in some embodiments, in the RNA 5-methylcytidine is substituted partially or completely, preferably completely, for cytidine. In some embodiments, the RNA comprises 5-methylcytidine and one or more selected from pseudouridine (ψ), N1-methyl-pseudouridine (m1ψ), and 5-methyl-uridine (m5U). In some embodiments, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments, the RNA according to the present disclosure comprises a 5′-cap. In some embodiments, the RNA of the present disclosure does not have uncapped 5′-triphosphates. In some embodiments, the RNA may be modified by a 5′-cap analog. The term “5′-cap” refers to a structure found on the 5′-end of an mRNA molecule and generally consists of a guanosine nucleotide connected to the mRNA via a 5′- to 5′-triphosphate linkage. In some embodiments, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the building block cap for RNA is m₂^{7,3′-O}Gppp(m₁^{2′-O})ApG (also sometimes referred to as m₂^{7,3′-O}G(5′)ppp(5′)m^{2′-O}ApG), which has the following structure:

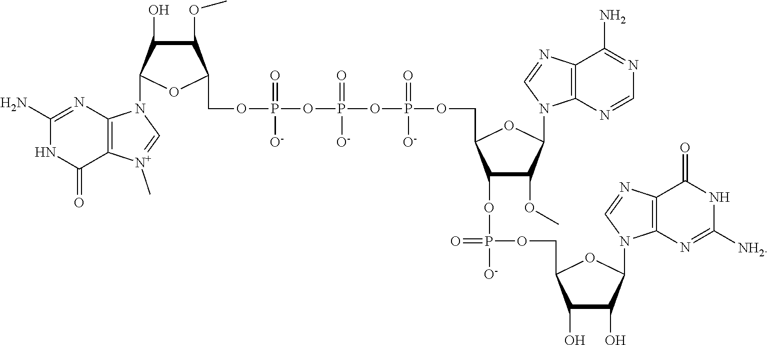

Below is an exemplary Cap1 RNA, which comprises RNA and m₂^{7,3′-O}G(5′)ppp(5′)m^{2′-O}ApG:

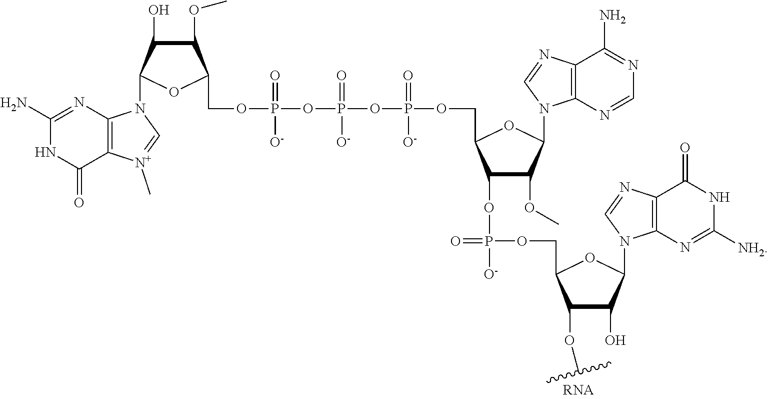

Below is another exemplary Cap1 RNA (no cap analog):

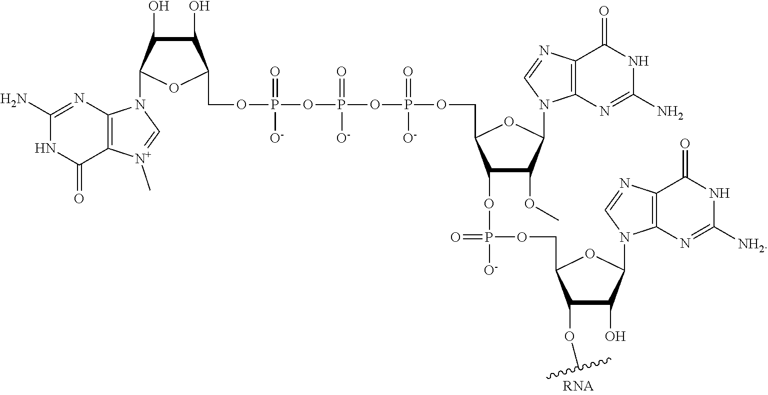

In some embodiments, the RNA is modified with “Cap0” structures using, in some embodiments, the cap analog anti-reverse cap (ARCA Cap (m₂^{7,3′-O}G(5′)ppp(5′)G)) with the structure:

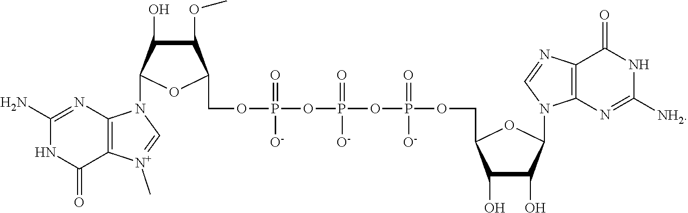

Below is an exemplary Cap0 RNA comprising RNA and m₂^{7,3′-O}G(5′)ppp(5′)G:

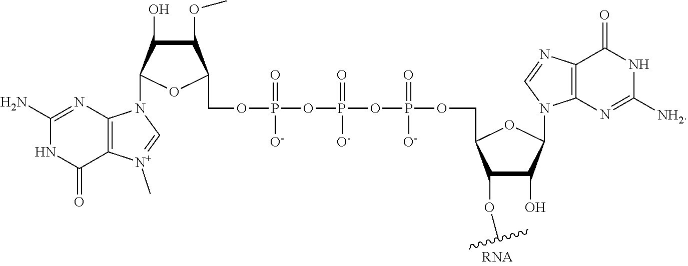

In some embodiments, the “Cap0” structures are generated using the cap analog Beta-S-ARCA (m₂^{7,3′-O}G(5′)ppSp(5′)G) with the structure:

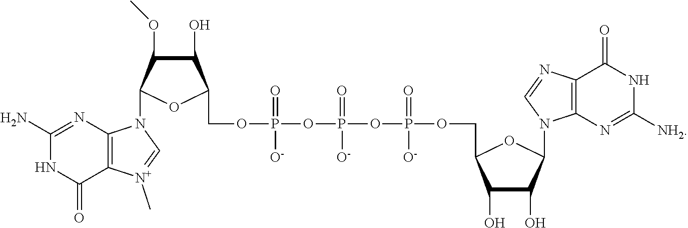

Below is an exemplary Cap0 RNA comprising Beta-S-ARCA (m₂^{7,2′-O}G(5′)ppSp(5′)G) and RNA:

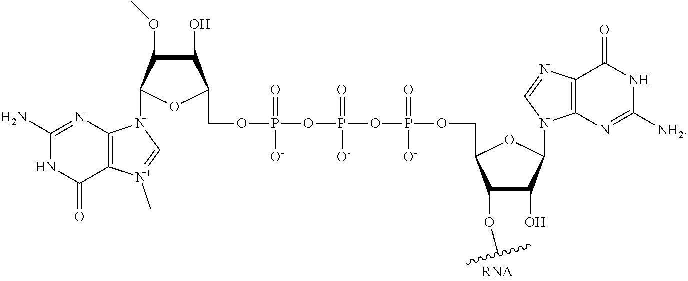

The “D1” diastereomer of beta-S-ARCA or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time (cf., WO 2011/015347, herein incorporated by reference).

A particularly preferred cap is m₂^{7,3′-O}Gppp(m₁^{2′-O})ApG. In some embodiments, the RNA sequences described herein (e.g., SEQ ID NO: 5 and/or 7) are capped with m₂^{7,3′-O}Gppp(m₁^{2′-O})ApG. In some embodiments, the ApG of the cap corresponds to the two 5′ nucleotides of the RNA sequences described herein.

In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g. directly adjacent to the poly(A) sequence.

In some embodiments, RNA comprises a 5′-UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.

In some embodiments, RNA comprises a 3′-UTR comprising the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9.

A particularly preferred 5′-UTR comprises the nucleotide sequence of SEQ ID NO: 8. A particularly preferred 3′-UTR comprises the nucleotide sequence of SEQ ID NO: 9.

In some embodiments, the RNA according to the present disclosure comprises a 3′-poly(A) sequence.

As used herein, the term “poly(A) sequence” or “poly-A tail” refers to an uninterrupted or interrupted sequence of adenylate residues which is typically located at the 3′-end of an RNA molecule. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017).

The poly(A) sequence may be of any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette. In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly(A) sequence contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, RNA comprises a poly(A) sequence comprising the nucleotide sequence of SEQ ID NO: 10, or a nucleotide sequence having at least 99%, 98%, 97%, 96%, 95%, 90%, 85%, or 80% identity to the nucleotide sequence of SEQ ID NO: 10.

A particularly preferred poly(A) sequence comprises the nucleotide sequence of SEQ ID NO: 10. According to the disclosure, a binding agent is preferably administered as single-stranded, 5′-capped mRNA that is translated into the respective protein upon entering cells of a subject being administered the RNA. Preferably, the RNA contains structural elements optimized for maximal efficacy of the RNA with respect to stability and translational efficiency (5′-cap, 5′-UTR, 3′-UTR, poly(A) sequence).

In some embodiments, m₂^{7,3′-O}Gppp(m₁^{2′-O})ApG is utilized as specific capping structure at the 5′-end of the RNA. In some embodiments, the 5′-UTR sequence is derived from the human alpha-globin mRNA and optionally has an optimized ‘Kozak sequence’ to increase translational efficiency. In some embodiments, a combination of two sequence elements (FI element) derived from the “amino terminal enhancer of split” (AES) mRNA (called F) and the mitochondrial encoded 12S ribosomal RNA (called I) are placed between the coding sequence and the poly(A) sequence to assure higher maximum protein levels and prolonged persistence of the mRNA. These were identified by an ex vivo selection process for sequences that confer RNA stability and augment total protein expression (see WO 2017/060314, herein incorporated by reference). In some embodiments, a poly(A) sequence measuring 110 nucleotides in length, consisting of a stretch of 30 adenosine residues, followed by a 10 nucleotide linker sequence and another 70 adenosine residues is used. This poly(A) sequence was designed to enhance RNA stability and translational efficiency.

In some embodiments of all aspects of the invention, RNA encoding a binding agent is expressed in cells, e.g., liver cells, of the subject treated to provide the binding agent. In some embodiments of all aspects of the invention, the RNA is transiently expressed in cells of the subject. In some embodiments of all aspects of the invention, the RNA is in vitro transcribed RNA. In some embodiments of all aspects of the invention, expression of the binding agent is into the extracellular space, i.e., the binding agent is secreted. In some embodiments of all aspects of the invention, expression of the binding agent is into the blood stream.

In the context of the present disclosure, the term “transcription” relates to a process, wherein the genetic code in a DNA sequence is transcribed into RNA. Subsequently, the RNA may be translated into peptide or protein.

According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”. According to the present invention, the RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

With respect to RNA, the term “expression” or “translation” relates to the process in the ribosomes of a cell by which a strand of mRNA directs the assembly of a sequence of amino acids to make a peptide or protein.

In some embodiments, after administration (e.g., intravenous administration) of the RNA described herein, e.g., formulated as RNA lipid particles, at least a portion of the RNA is delivered to target cells (e.g., liver cells). In some embodiments, at least a portion of the RNA is delivered to the cytosol of the target cells. In some embodiments, the RNA is translated by the target cells to produce the peptide or protein it enodes. Accordingly, the present disclosure also relates to a method for delivering RNA to target cells in a subject comprising the administration of the RNA particles described herein to the subject. In some embodiments, the RNA is delivered to the cytosol of the target cells. In some embodiments, the RNA is translated by the target cells to produce the peptide or protein encoded by the RNA.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In some embodiments, the RNA encoding binding agent to be administered according to the invention is non-immunogenic.

The term “non-immunogenic RNA” as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In one preferred embodiment, non-immunogenic RNA, which is also termed modified RNA (modRNA) herein, is rendered non-immunogenic by incorporating modified nucleosides suppressing RNA-mediated activation of innate immune receptors into the RNA and removing double-stranded RNA (dsRNA).

For rendering the non-immunogenic RNA non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In some embodiments, the modified nucleosides comprises a replacement of one or more uridines with a nucleoside comprising a modified nucleobase. In some embodiments, the modified nucleobase is a modified uracil. In some embodiments, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmoSU), uridine 5-oxyacetic acid methyl ester (mcmoSU), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine. In one particularly preferred embodiment, the nucleoside comprising a modified nucleobase is pseudouridine (ψ), N1-methyl-pseudouridine (m1ψ) or 5-methyl-uridine (m5U), in particular N1-methyl-pseudouridine.

In some embodiments, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of mRNA by in vitro transcription (IVT), e.g., using T7 RNA polymerase, significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In some embodiments, an RNA preparation is contacted with a cellulose material and the ssRNA is separated from the cellulose material under conditions which allow binding of dsRNA to the cellulose material and do not allow binding of ssRNA to the cellulose material.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In some embodiments, the removal of dsRNA from non-immunogenic RNA comprises a removal of dsRNA such that less than 10%, less than 5%, less than 4%, less than 3%, less than 2%, less than 1%, less than 0.5%, less than 0.3%, less than 0.1%, or less than 0.01% of the RNA in the non-immunogenic RNA composition is dsRNA. In some embodiments, the non-immunogenic RNA is free or essentially free of dsRNA. In some embodiments, the non-immunogenic RNA composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

In some embodiments, the non-immunogenic RNA is translated in a cell more efficiently than standard RNA with the same sequence. In some embodiments, translation is enhanced by a factor of 2-fold relative to its unmodified counterpart. In some embodiments, translation is enhanced by a 3-fold factor. In some embodiments, translation is enhanced by a 4-fold factor. In some embodiments, translation is enhanced by a 5-fold factor. In some embodiments, translation is enhanced by a 6-fold factor. In some embodiments, translation is enhanced by a 7-fold factor. In some embodiments, translation is enhanced by an 8-fold factor. In some embodiments, translation is enhanced by a 9-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1,000-fold factor. In some embodiments, translation is enhanced by a 2,000-fold factor. In some embodiments, the factor is 10-1,000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1,000-fold. In some embodiments, the factor is 30-1,000-fold. In some embodiments, the factor is 50-1,000-fold. In some embodiments, the factor is 100-1,000-fold. In some embodiments, the factor is 200-1,000-fold. In some embodiments, translation is enhanced by any other significant amount or range of amounts.

In some embodiments, the non-immunogenic RNA exhibits significantly less innate immunogenicity than standard RNA with the same sequence. In some embodiments, the non-immunogenic RNA exhibits an innate immune response that is 2-fold less than its unmodified counterpart. In some embodiments, innate immunogenicity is reduced by a 3-fold factor. In some embodiments, innate immunogenicity is reduced by a 4-fold factor. In some embodiments, innate immunogenicity is reduced by a 5-fold factor. In some embodiments, innate immunogenicity is reduced by a 6-fold factor. In some embodiments, innate immunogenicity is reduced by a 7-fold factor. In some embodiments, innate immunogenicity is reduced by an 8-fold factor. In some embodiments, innate immunogenicity is reduced by a 9-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1,000-fold factor. In some embodiments, innate immunogenicity is reduced by a 2,000-fold factor.

The term “exhibits significantly less innate immunogenicity” refers to a detectable decrease in innate immunogenicity. In some embodiments, the term refers to a decrease such that an effective amount of the non-immunogenic RNA can be administered without triggering a detectable innate immune response. In some embodiments, the term refers to a decrease such that the non-immunogenic RNA can be repeatedly administered without eliciting an innate immune response sufficient to detectably reduce production of the protein encoded by the non-immunogenic RNA. In some embodiments, the decrease is such that the non-immunogenic RNA can be repeatedly administered without eliciting an innate immune response sufficient to eliminate detectable production of the protein encoded by the non-immunogenic RNA. “Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked,” “fused”, or “fusion” are used interchangeably. These terms refer to the joining together of two or more elements or components or domains.

### Codon-Optimization/Increase in G/C Content

In some embodiments, the amino acid sequence of a binding agent described herein is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In some embodiments, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present invention, coding regions are preferably codon-optimized for optimal expression in a subject to be treated using the RNA molecules described herein. Codon-optimization is based on the finding that the translation efficiency is also determined by a different frequency in the occurrence of tRNAs in cells. Thus, the sequence of RNA may be modified such that codons for which frequently occurring tRNAs are available are inserted in place of “rare codons”.

In some embodiments of the invention, the guanosine/cytosine (G/C) content of the coding region of the RNA described herein is increased compared to the G/C content of the corresponding coding sequence of the wild type RNA, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favourable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides. In various embodiments, the G/C content of the coding region of the RNA described herein is increased by at least 10%, at least 20%, at least 30%, at least 40%, at least 50%, at least 55%, or even more compared to the G/C content of the coding region of the wild type RNA.

### Nucleic Acid Containing Particles

Nucleic acids described herein such as RNA encoding a binding agent may be administered formulated as particles.

In the context of the present disclosure, the term “particle” relates to a structured entity formed by molecules or molecule complexes.

In some embodiments, the particles described herein are nanoparticles. The term “nanoparticle” relates to a nano-sized particle, wherein all three external dimensions of the particle are in the nanoscale, i.e., at least about 1 nm and below about 1,000 nm. Preferably, the size of a particle is its diameter.

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features,

In a particulate formulation comprising RNA, e.g., first RNA and second RNA, it is possible that each RNA species is separately formulated as an individual particulate formulation. In that case, each individual particulate formulation will comprise one RNA species. The individual particulate formulations may be present as separate entities, e.g. in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations). In some embodiments, a composition such as a pharmaceutical composition comprises more than one individual particle formulation. Respective pharmaceutical compositions are referred to as mixed particulate formulations. Mixed particulate formulations according to the invention are obtainable by forming, separately, individual particulate formulations, followed by a step of mixing of the individual particulate formulations. By the step of mixing, a formulation comprising a mixed population of RNA-containing particles is obtainable. Individual particulate populations may be together in one container, comprising a mixed population of individual particulate formulations. Alternatively, it is possible that all RNA species of the pharmaceutical composition are formulated together as a combined particulate formulation. Such formulations are obtainable by providing a combined formulation (typically combined solution) of all RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

A “nucleic acid particle” can be used to deliver nucleic acid to a target site of interest (e.g., cell, tissue, organ, and the like). A nucleic acid particle may be formed from nucleic acid and at least one particle forming component, e.g., at least one cationic or cationically ionizable lipid or lipid-like material, at least one cationic polymer such as protamine, or a mixture thereof. Electrostatic interactions between positively charged molecules such as polymers and lipids and negatively charged nucleic acid are involved in particle formation. This results in complexation and spontaneous formation of nucleic acid particles. Without intending to be bound by any theory, it is believed that the cationic or cationically ionizable lipid or lipid-like material and/or the cationic polymer combine together with the nucleic acid to form aggregates, and this aggregation results in colloidally stable particles. Nucleic acid particles include lipid nanoparticle (LNP)-based and lipoplex (LPX)-based formulations.

The term “colloid” as used herein relates to a type of homogeneous mixture in which dispersed particles do not settle out. The insoluble particles in the mixture are microscopic, with particle sizes between 1 and 1,000 nanometers. The mixture may be termed a colloid or a colloidal suspension. Sometimes the term “colloid” only refers to the particles in the mixture and not the entire suspension.

In some embodiments, particles described herein further comprise at least one lipid or lipid-like material other than a cationic or cationically ionizable lipid or lipid-like material, at least one polymer other than a cationic polymer, or a mixture thereof.

Nucleic acid particles described herein may have an average diameter that in some embodiments ranges from about 30 nm to about 1,000 nm, from about 50 nm to about 800 nm, from about 70 nm to about 600 nm, from about 90 nm to about 400 nm, or from about 100 nm to about 300 nm.

The term “average diameter” refers to the mean hydrodynamic diameter of particles as measured by dynamic laser light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

Nucleic acid particles described herein may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

The N/P ratio gives the ratio of the nitrogen groups in the lipid to the number of phosphate groups in the nucleic acid, e.g., RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells.

Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

### Cationic Polymer

Given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties such as those described herein.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In some embodiments, the polymer is biocompatible. Biocompatible polymers are polymers that typically do not result in significant cell death at moderate concentrations. In some embodiments, the biocompatible polymer is biodegradable, i.e., the polymer is able to degrade, chemically and/or biologically, within a physiological environment, such as within the body. In some embodiments, polymer may be protamine or polyalkyleneimine, in particular protamine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In some embodiments, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75·10² to 107 Da, preferably 1,000 to 105 Da, more preferably 10,000 to 40,000 Da, more preferably 15,000 to 30,000 Da, even more preferably 20,000 to 25,000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind nucleic acid. In some embodiments, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipid and Lipid-Like Material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances, in particular amphiphilic substances, that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids are generally cylindrical. The area occupied by the two alkyl chains is similar to the area occupied by the polar head group. Lipids have low solubility as monomers and tend to aggregate into planar bilayers that are water insoluble. Traditional surfactant monomers are generally cone shaped. The hydrophilic head groups tend to occupy more molecular space than the linear alkyl chains. Surfactants tend to aggregate into spherical or elliptoid micelles that are water soluble. While lipids also have the same general structure as surfactants—a polar hydrophilic head group and a nonpolar hydrophobic tail—lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In some embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids or Lipid-Like Materials

The nucleic acid particles described herein may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In some embodiments, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In some embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid or lipid-like material” unless contradicted by the circumstances.

In some embodiments, the cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy)propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200). In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

### Additional Lipids or Lipid-Like Materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In some embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In preferred embodiments, the additional lipid comprises one of the following neutral lipid components, e.g., neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In some embodiments, the nucleic acid particles include both a cationic lipid and an additional lipid.

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

### Polymer-Conjugated Lipids

In some embodiments, a particle may comprise at least one polymer-conjugated lipid. A polymer-conjugated lipid is typically a molecule comprising a lipid portion and a polymer portion conjugated thereto. In some embodiments, a polymer-conjugated lipid is a PEG-conjugated lipid, also referred to herein as pegylated lipid or PEG-lipid.

In some embodiments, a polymer-conjugated lipid is designed to sterically stabilize a lipid particle by forming a protective hydrophilic layer that shields the hydrophobic lipid layer. In some embodiments, a polymer-conjugated lipid can reduce its association with serum proteins and/or the resulting uptake by the reticuloendothelial system when such lipid particles are administered in vivo.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω methoxy(polyethoxy)ethyl)carbamate, and the like.

In some embodiments, a particle may comprise one or more PEG-conjugated lipids or pegylated lipids as described in WO 2017/075531 and WO 2018/081480, the entire contents of each of which are incorporated herein by reference for the purposes described herein.

### Lipoplex Particles

In some embodiments of the present disclosure, the RNA described herein may be present in RNA lipoplex particles.

Lipoplexes (LPX) are electrostatic complexes which are generally formed by mixing preformed cationic lipid liposomes with anionic RNA. Formed lipoplexes possess distinct internal arrangements of molecules that arise due to the transformation from liposomal structure into compact RNA-lipoplexes. These formulations are generally characterized by their poor encapsulation of the nucleic acid and incomplete entrapment of the nucleic acid.

Liposomes are spherical vesicles comprising unilamellar or multilamellar phospholipid bilayers enclosing an aqueous core. They are prepared from materials possessing polar head (hydrophilic) groups and nonpolar tail (hydrophobic) groups. The interaction between these groups induces the formation of vesicles. Cationic lipids employed in formulating liposomes designed for the delivery of nucleic acids are amphiphilic in nature and consist of a positively charged (cationic) amine head group linked to a hydrocarbon chain or cholesterol derivative via glycerol.

Positively charged liposomes may be generally synthesized using a cationic lipid, such as DOTMA, and additional lipids, such as DOPE. In some embodiments, a RNA lipoplex particle is a nanoparticle.

In some embodiments, the RNA lipoplex particles include both a cationic lipid and an additional lipid. In an exemplary embodiment, the cationic lipid is DOTMA and the additional lipid is DOPE. In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in some embodiments ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The RNA lipoplex particles may be prepared using liposomes that may be obtained by injecting a solution of the lipids in ethanol into water or a suitable aqueous phase. In some embodiments, the aqueous phase has an acidic pH. In some embodiments, the aqueous phase comprises acetic acid, e.g., in an amount of about 5 mM. Liposomes may be used for preparing RNA lipoplex particles by mixing the liposomes with RNA.

In some embodiments, the liposomes and RNA lipoplex particles comprise at least one cationic lipid and at least one additional lipid. In some embodiments, the at least one cationic lipid comprises 1,2-di-O-octadecenyl-3-trimethylammonium propane (DOTMA) and/or 1,2-dioleoyl-3-trimethylammonium-propane (DOTAP). In some embodiments, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In some embodiments, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In some embodiments, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

### Lipid Nanoparticles (LNPs)

In some embodiments, RNA described herein is present in the form of lipid nanoparticles (LNPs). The LNP may comprise any lipid capable of forming a particle to which the one or more nucleic acid molecules are attached, or in which the one or more nucleic acid molecules are encapsulated. LNPs typically comprise four components: ionizable cationic lipids, neutral lipids such as phospholipids, a steroid such as cholesterol, and a polymer-conjugated lipid such as PEG-lipid. LNPs may be prepared by mixing lipids dissolved in ethanol with nucleic acid in an aqueous buffer. In some embodiments, in the RNA LNPs described herein the mRNA is bound by ionizable lipid that occupies the central core of the LNP. PEG lipid forms the surface of the LNP, along with phospholipids. In some embodiments, the surface comprises a bilayer. In some embodiments, cholesterol and ionizable lipid in charged and uncharged forms can be distributed throughout the LNP.

In some embodiments, the LNP comprises one or more cationic lipids, and one or more stabilizing lipids. Stabilizing lipids include neutral lipids and pegylated lipids.

In some embodiments, the LNP comprises a cationic lipid, a neutral lipid, a steroid, a polymer-conjugated lipid; and the RNA, encapsulated within or associated with the lipid nanoparticle.

In some embodiments, the LNP comprises from 40 to 55 mol percent, from 40 to 50 mol percent, from 41 to 50 mol percent, from 42 to 50 mol percent, from 43 to 50 mol percent, from 44 to 50 mol percent, from 45 to 50 mol percent, from 46 to 50 mol percent, from 46 to 49 mol percent, or about 47 or 48 mol percent of the cationic lipid. In some embodiments, the LNP comprises about 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, or 49 mol percent of the cationic lipid.

In some embodiments, the neutral lipid is present in a concentration ranging from 5 to 15 mol percent, from 7 to 13 mol percent, or from 9 to 11 mol percent. In some embodiments, the neutral lipid is present in a concentration of about 10 mol percent.

In some embodiments, the steroid is present in a concentration ranging from 30 to 50 mol percent, from 35 to 45 mol percent or from 38 to 43 mol percent. In some embodiments, the steroid is present in a concentration of about 41 mol percent.

In some embodiments, the LNP comprises from 1 to 10 mol percent, from 1 to 5 mol percent, or from 1 to 2.5 mol percent of the polymer-conjugated lipid. In some embodiments, the polymer-conjugated lipid is present in a concentration of about 1.5, 1.6, 1.7, 1.8, 1.9 or 2.0 mol percent.

In some embodiments, the LNP comprises from 45 to 50 mol percent a cationic lipid; from 5 to 15 mol percent of a neutral lipid; from 35 to 45 mol percent of a steroid; from 1 to 5 mol percent of a polymer-conjugated lipid; and the RNA, encapsulated within or associated with the lipid nanoparticle.

In some embodiments, the mol percent is determined based on total mol of lipid present in the lipid nanoparticle. In some embodiments, the mol percent is determined based on total mol of cationic lipid, neutral lipid, steroid and polymer-conjugated lipid present in the lipid nanoparticle. In some embodiments, the neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, the steroid is cholesterol.

In some embodiments, the polymer conjugated lipid is a pegylated lipid. In some embodiments, the pegylated lipid has the following structure:

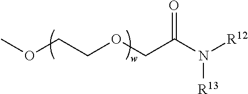

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R¹² and R¹³ are each independently straight, saturated alkyl chains containing from 12 to 16 carbon atoms. In some embodiments, w has a mean value ranging from 40 to 55. In some embodiments, the average w is about 45. In some embodiments, R¹² and R¹³ are each independently a straight, saturated alkyl chain containing about 14 carbon atoms, and w has a mean value of about 45.

In some embodiments, a pegylated lipid is or comprises 2-[(Polyethylene glycol)-2000]-N,N-ditetradecylacetamide.

In some embodiments, the cationic lipid component of the LNPs has the structure of Formula (III):

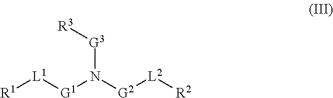

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

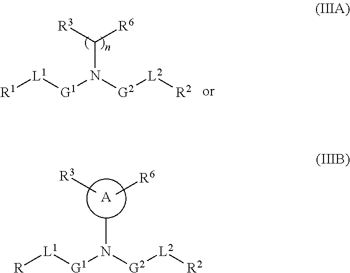

wherein: A is a 3 to 8-membered cycloalkyl or cycloalkylene ring; R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

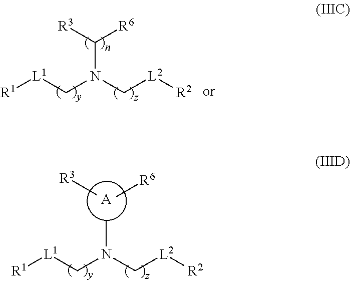

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

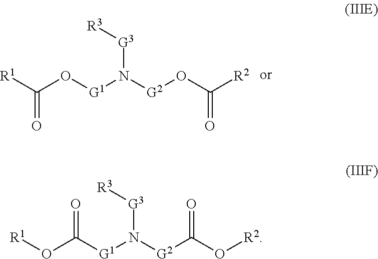

In some of the foregoing embodiments of Formula (III), the lipid has one of the following structures (IIIG), (IIIH), (IIII), or (IIIJ):

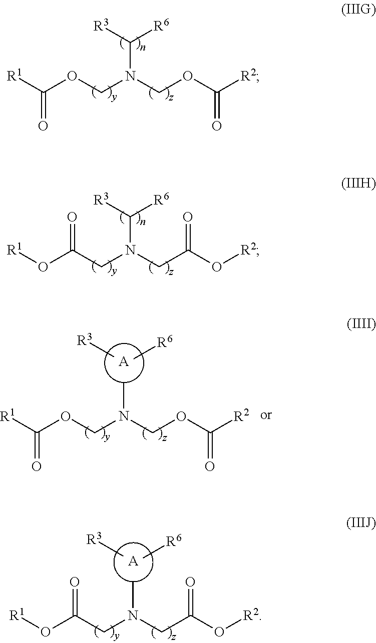

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

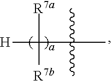

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl. In different embodiments of Formula (III), R¹ or R², or both, has one of the following structures:

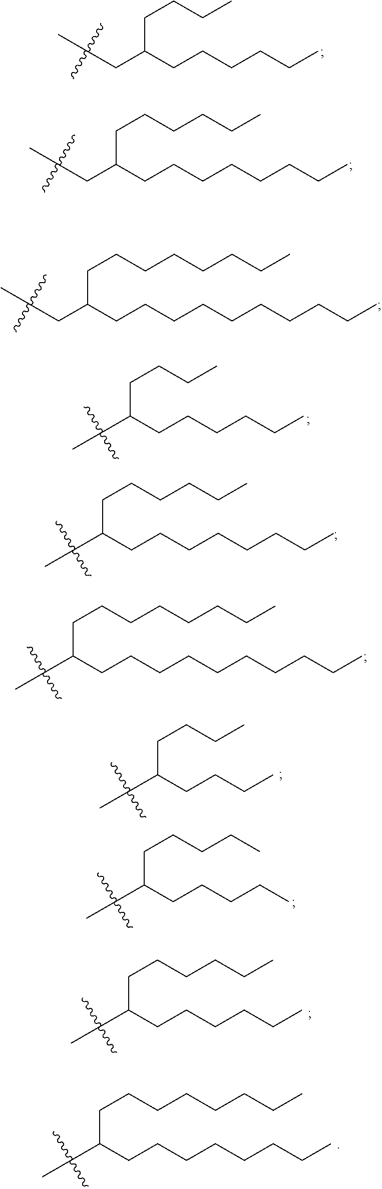

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in the table below.

### Representative Compounds of Formula (III).

| No. | Structure |
|---|---|
| III-1 | 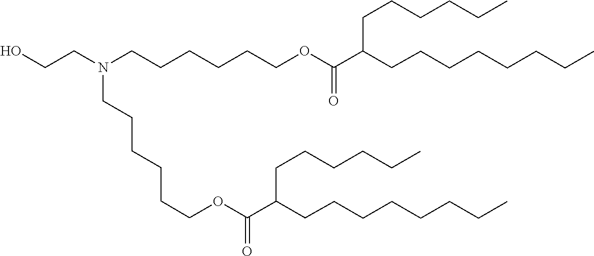 [SMILES: CCCCCCCCC(CCCCCC)C(=O)OCCCCCCN(CCO)CCCCCCOC(=O)C(CCCCCC)CCCCCCCC] |
| III-2 | 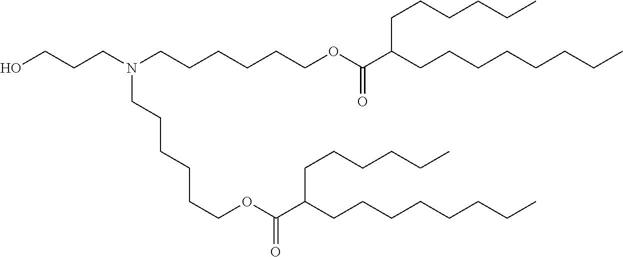 [SMILES: CCCCCCCCC(CCCCCC)C(=O)OCCCCCCN(CCCO)CCCCCCOC(=O)C(CCCCCC)CCCCCCCC] |
| III-3 | 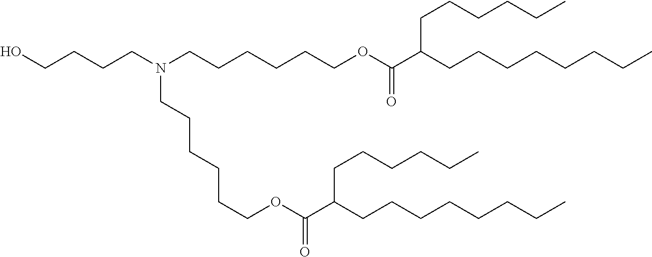 [SMILES: CCCCCCCCC(CCCCCC)C(=O)OCCCCCCN(CCCCO)CCCCCCOC(=O)C(CCCCCC)CCCCCCCC] |
| III-4 | 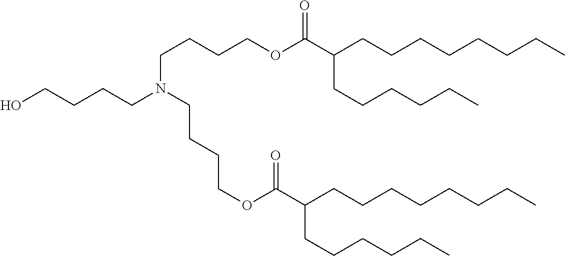 [SMILES: CCCCCCCCC(CCCCCC)C(=O)OCCCCN(CCCCO)CCCCOC(=O)C(CCCCCC)CCCCCCCC] |
| III-5 | 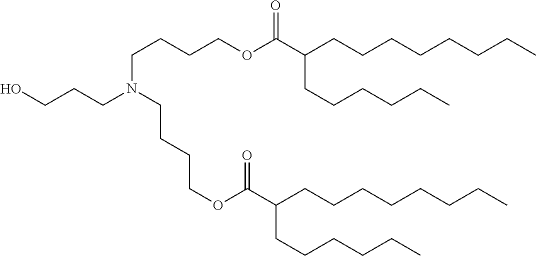 [SMILES: CCCCCCCCC(CCCCCC)C(=O)OCCCCN(CCCO)CCCCOC(=O)C(CCCCCC)CCCCCCCC] |
| III-6 | 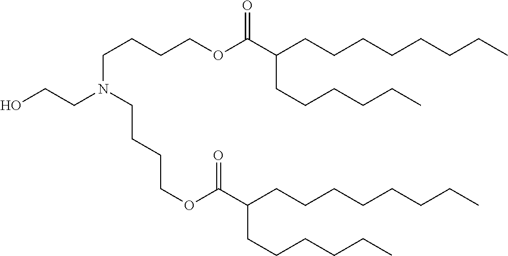 [SMILES: CCCCCCCCC(CCCCCC)C(=O)OCCCCN(CCO)CCCCOC(=O)C(CCCCCC)CCCCCCCC] |
| III-7 | 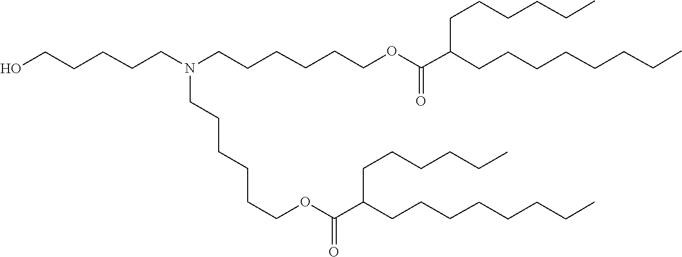 [SMILES: CCCCCCCCC(CCCCCC)C(=O)OCCCCCCN(CCCCCO)CCCCCCOC(=O)C(CCCCCC)CCCCCCCC] |
| III-8 | 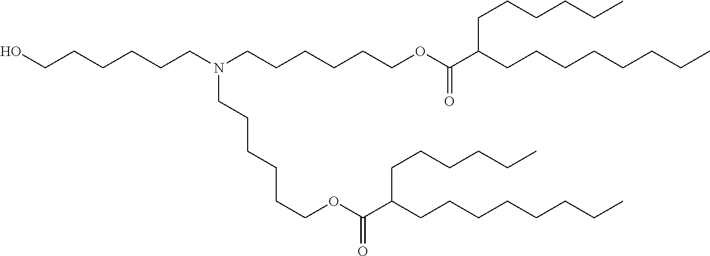 [SMILES: CCCCCCCCC(CCCCCC)C(=O)OCCCCCCN(CCCCCCO)CCCCCCOC(=O)C(CCCCCC)CCCCCCCC] |
| III-9 | 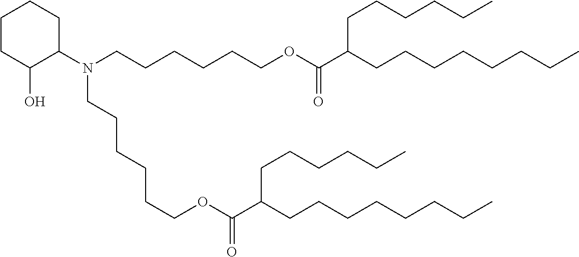 [SMILES: CCCCCCCCC(CCCCCC)C(=O)OCCCCCCN(CCCCCCOC(=O)C(CCCCCC)CCCCCCCC)C1CCCCC1O] |
| III-10 | 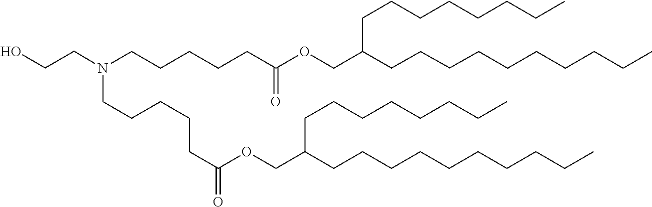 [SMILES: CCCCCCCCCCC(CCCCCCCC)COC(=O)CCCCCN(CCO)CCCCCC(=O)OCC(CCCCCCCC)CCCCCCCCCC] |
| III-11 | 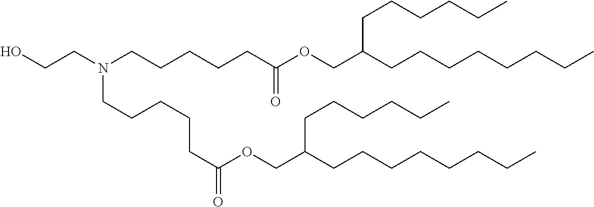 [SMILES: CCCCCCCCC(CCCCCC)COC(=O)CCCCCN(CCO)CCCCCC(=O)OCC(CCCCCC)CCCCCCCC] |
| III-12 | 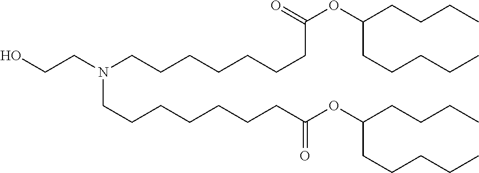 [SMILES: CCCCCC(CCCC)OC(=O)CCCCCCCN(CCO)CCCCCCCC(=O)OC(CCCC)CCCCC] |
| III-13 | 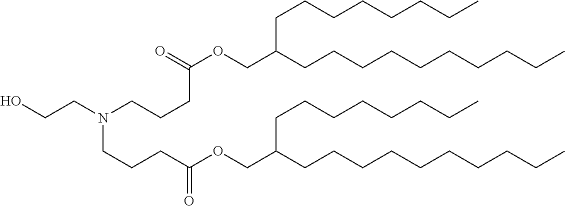 [SMILES: CCCCCCCCCCC(CCCCCCCC)COC(=O)CCCN(CCO)CCCC(=O)OCC(CCCCCCCC)CCCCCCCCCC] |
| III-14 | 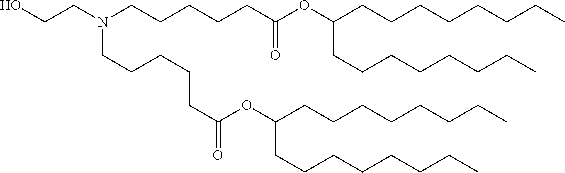 [SMILES: CCCCCCCCC(CCCCCCCC)OC(=O)CCCCCN(CCO)CCCCCC(=O)OC(CCCCCCCC)CCCCCCCC] |
| III-15 | 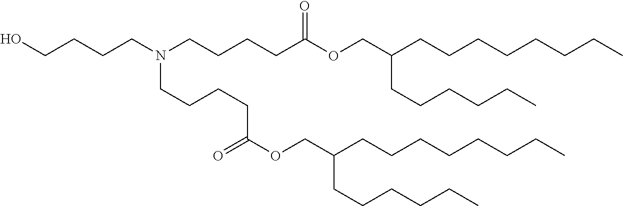 [SMILES: CCCCCCCCC(CCCCCC)COC(=O)CCCCN(CCCCO)CCCCC(=O)OCC(CCCCCC)CCCCCCCC] |
| III-16 | 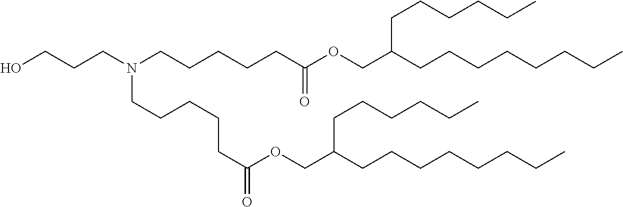 [SMILES: CCCCCCCCC(CCCCCC)COC(=O)CCCCCN(CCCO)CCCCCC(=O)OCC(CCCCCC)CCCCCCCC] |
| III-17 | 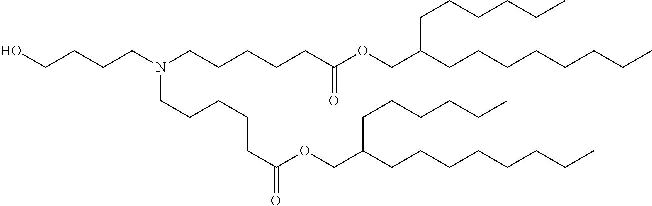 [SMILES: CCCCCCCCC(CCCCCC)COC(=O)CCCCCN(CCCCO)CCCCCC(=O)OCC(CCCCCC)CCCCCCCC] |
| III-18 | 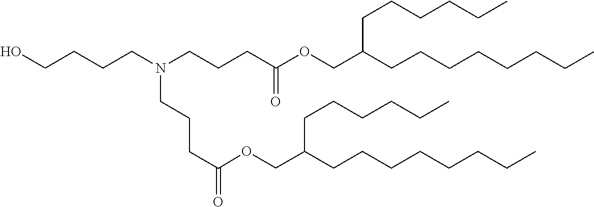 [SMILES: CCCCCCCCC(CCCCCC)COC(=O)CCCN(CCCCO)CCCC(=O)OCC(CCCCCC)CCCCCCCC] |
| III-19 | 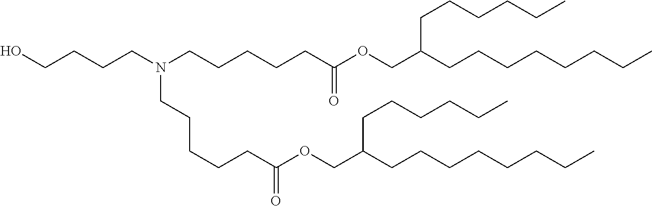 [SMILES: CCCCCCCCC(CCCCCC)COC(=O)CCCCCN(CCCCO)CCCCCC(=O)OCC(CCCCCC)CCCCCCCC] |
| III-20 | 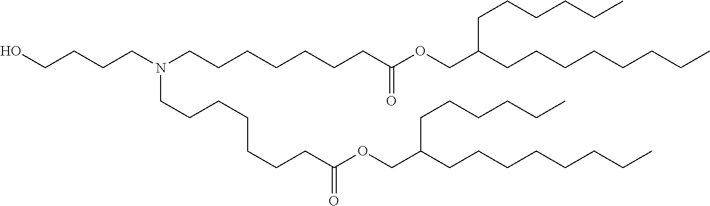 [SMILES: CCCCCCCCC(CCCCCC)COC(=O)CCCCCCCN(CCCCO)CCCCCCCC(=O)OCC(CCCCCC)CCCCCCCC] |
| III-21 | 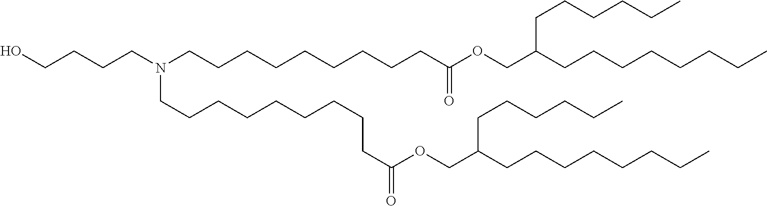 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 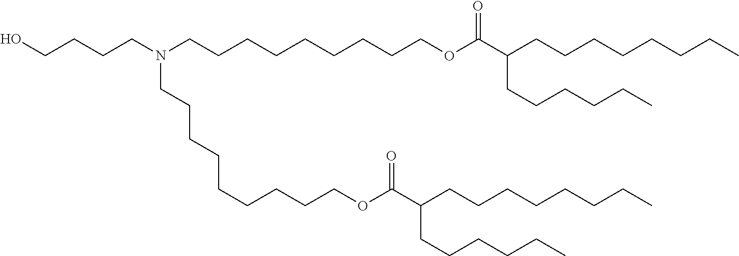 [SMILES: CCCCCCCCC(CCCCCC)C(=O)OCCCCCCCCCN(CCCCO)CCCCCCCCCOC(=O)C(CCCCCC)CCCCCCCC] |
| III-23 | 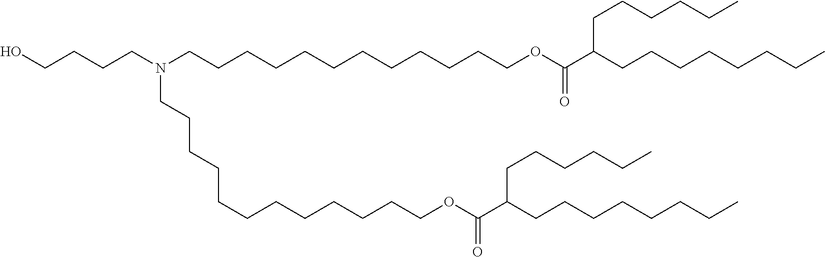 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 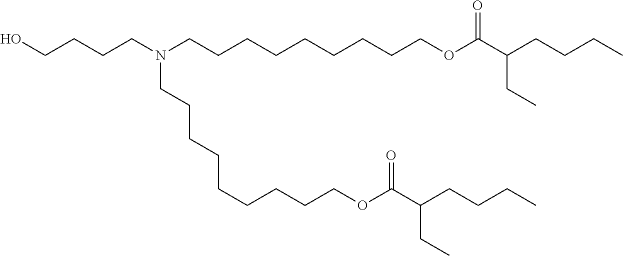 [SMILES: CCCCC(CC)C(=O)OCCCCCCCCCN(CCCCO)CCCCCCCCCOC(=O)C(CC)CCCC] |
| III-25 | 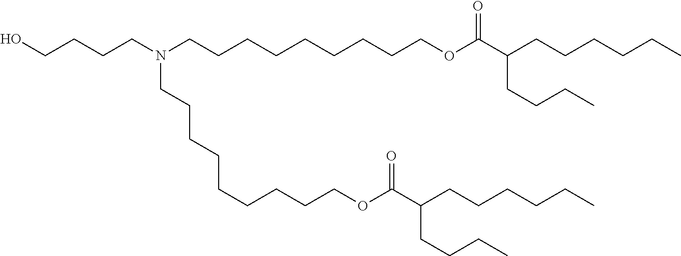 [SMILES: CCCCCCC(CCCC)C(=O)OCCCCCCCCCN(CCCCO)CCCCCCCCCOC(=O)C(CCCC)CCCCCC] |
| III-26 | 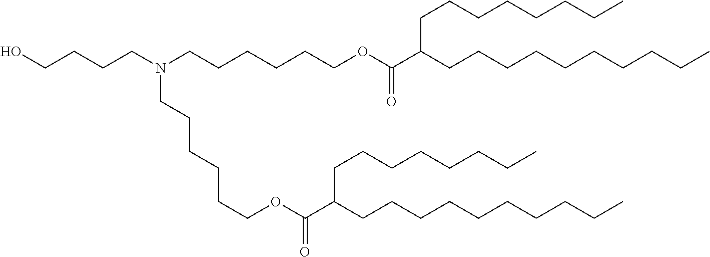 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 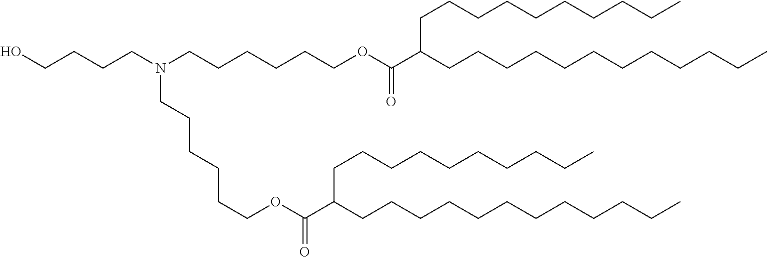 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 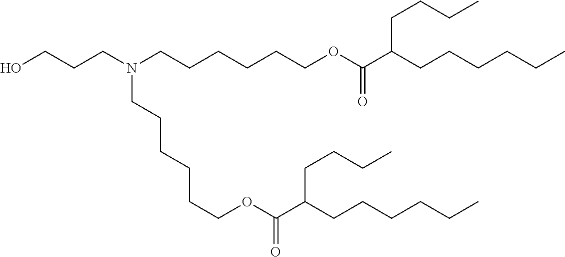 [SMILES: CCCCCCC(CCCC)C(=O)OCCCCCCN(CCCO)CCCCCCOC(=O)C(CCCC)CCCCCC] |
| III-29 | 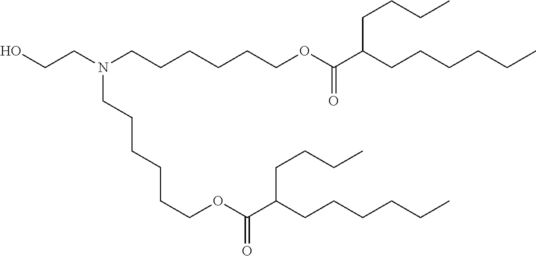 [SMILES: CCCCCCC(CCCC)C(=O)OCCCCCCN(CCO)CCCCCCOC(=O)C(CCCC)CCCCCC] |
| III-30 | 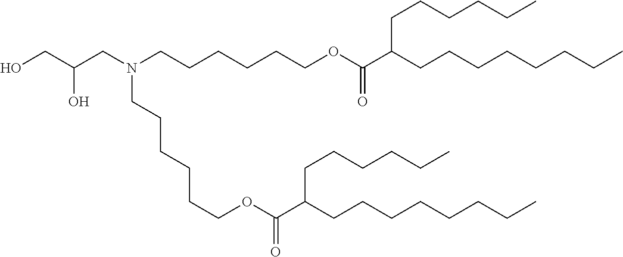 [SMILES: CCCCCCCCC(CCCCCC)C(=O)OCCCCCCN(CCCCCCOC(=O)C(CCCCCC)CCCCCCCC)CC(O)CO] |
| III-31 | 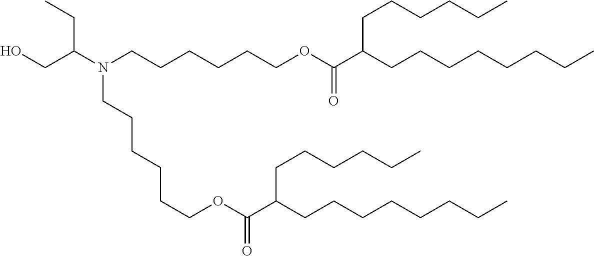 [SMILES: CCCCCCCCC(CCCCCC)C(=O)OCCCCCCN(CCCCCCOC(=O)C(CCCCCC)CCCCCCCC)C(CC)CO] |
| III-32 | 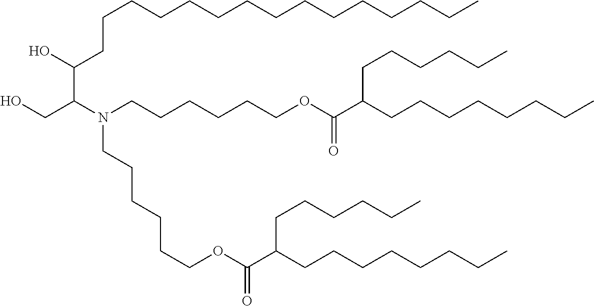 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 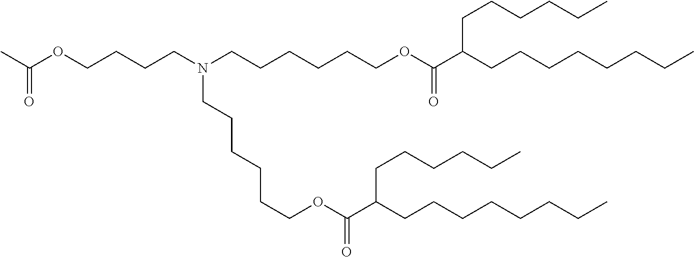 [SMILES: CCCCCCCCC(CCCCCC)C(=O)OCCCCCCN(CCCCCCOC(=O)C(CCCCCC)CCCCCCCC)CCCCOC(C)=O] |
| III-34 | 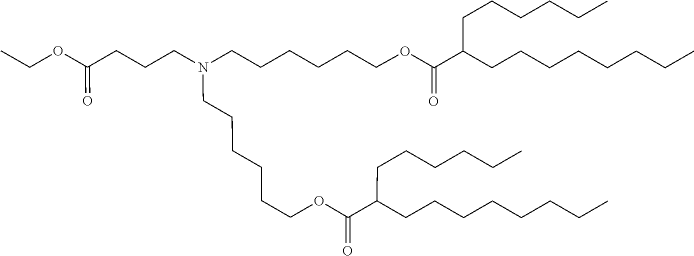 [SMILES: CCCCCCCCC(CCCCCC)C(=O)OCCCCCCN(CCCCCCOC(=O)C(CCCCCC)CCCCCCCC)CCCC(=O)OCC] |
| III-35 | 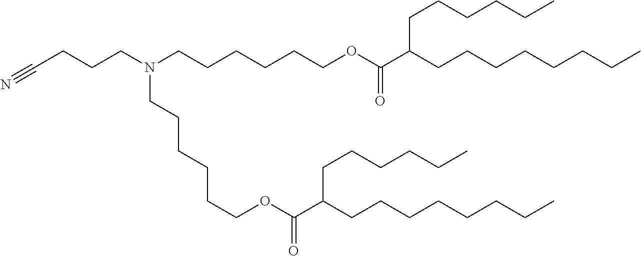 [SMILES: CCCCCCCCC(CCCCCC)C(=O)OCCCCCCN(CCCC#N)CCCCCCOC(=O)C(CCCCCC)CCCCCCCC] |
| III-36 | 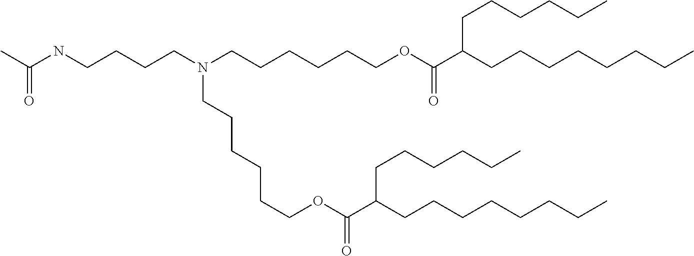 |
| III-37 | 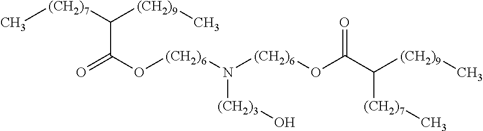 |
| III-38 | 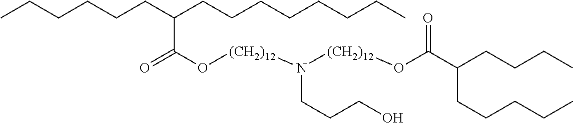 |
| III-39 | 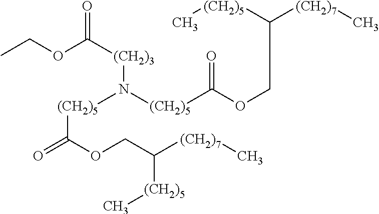 |
| III-40 | 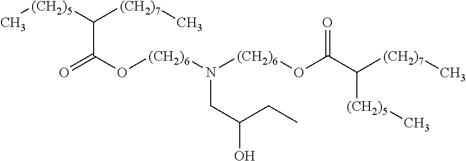 |
| III-41 | 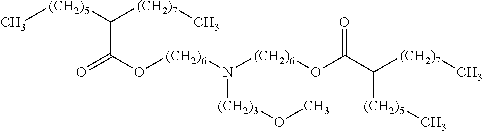 |
| III-42 | 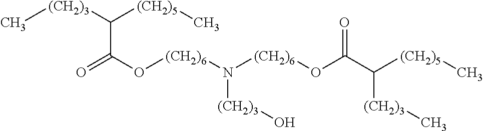 |
| III-43 | 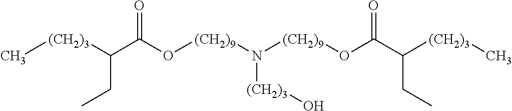 |
| III-44 | 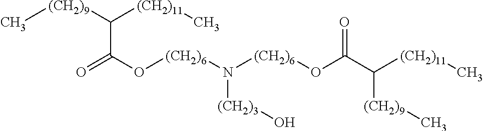 |
| III-45 | 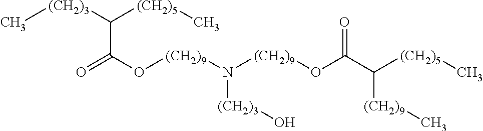 |
| III-46 | 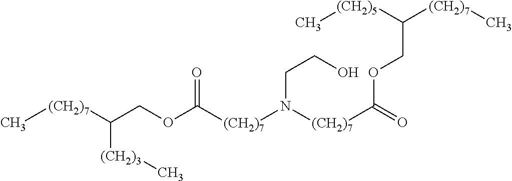 |
| III-47 | 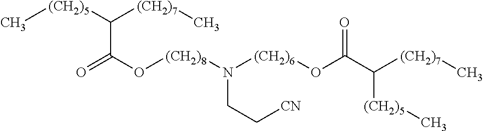 |
| III-48 | 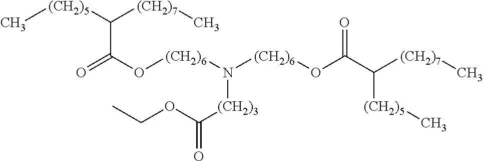 |
| III-49 | 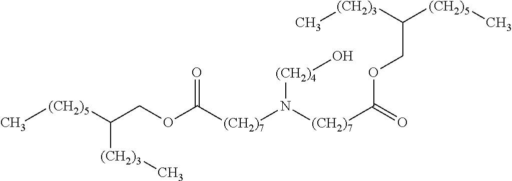 |

Various lipids (including, e.g., cationic lipids, neutral lipids, and polymer-conjugated lipids) are known in the art and can be used herein to form lipid nanoparticles, e.g., lipid nanoparticles targeting a specific cell type (e.g., liver cells). In some embodiments, a cationic lipid included in pharmaceutical compositions described herein may be ((3-hydroxypropyl)azanediyl)bis(nonane-9,1-diyl) bis(2-butyloctanoate) or a derivative thereof. In some embodiments, neutral lipid included in pharmaceutical compositions described herein may be or comprise a phospholipid or derivative thereof (e.g., 1,2-Distearoyl-sn-glycero-3-phosphocholine (DPSC)) and/or cholesterol. In some embodiments, a polymer-conjugated lipid included in pharmaceutical compositions described herein may be a PEG-conjugated lipid (e.g., 2-[(polyethylene glycol)-2000]-N,N-ditetradecylacetamide or a derivative thereof).

In some embodiments, the LNP comprises a lipid of Formula (III), RNA, a neutral lipid, a steroid and a pegylated lipid. In some embodiments, the lipid of Formula (III) is compound 111-45. In some embodiments, the neutral lipid is DSPC. In some embodiments, the steroid is cholesterol. In some embodiments, the pegylated lipid is ALC-0159.

### ALC-0159:

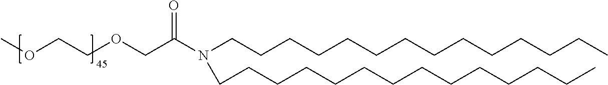

In some embodiments, the cationic lipid is present in the LNP in an amount from about 45 to about 50 mole percent. In some embodiments, the neutral lipid is present in the LNP in an amount from about 5 to about 15 mole percent. In some embodiments, the steroid is present in the LNP in an amount from about 35 to about 45 mole percent. In some embodiments, the pegylated lipid is present in the LNP in an amount from about 1 to about 5 mole percent.

In some embodiments, the LNP comprises compound 111-45 in an amount from about 45 to about 50 mole percent, DSPC in an amount from about 5 to about 15 mole percent, cholesterol in an amount from about 35 to about 45 mole percent, and ALC-0159 in an amount from about 1 to about 5 mole percent.

In some embodiments, the LNP comprises compound 111-45 in an amount of about 47 or 48 mole percent, DSPC in an amount of about 10 mole percent, cholesterol in an amount of about 41 mole percent, and ALC-0159 in an amount of about 1.6 or 1.7 mole percent.

The N/P value is preferably at least about 4. In some embodiments, the N/P value ranges from 4 to 20, 4 to 12, 4 to 10, 4 to 8, or 5 to 7. In some embodiments, the N/P value is about 6.

### Embodiments of Administered RNAs

In some embodiments, compositions or medical preparations described herein comprise RNA encoding a first polypeptide chain and RNA encoding a second polypeptide chain, said first and second polypeptide chains interacting with each other to form a binding agent described herein. Likewise, methods described herein comprise administration of such RNA. In some embodiments, the RNA is in-vitro transcribed RNA.

In some embodiments, the RNA is nucleoside-modified mRNA (modRNA). In some embodiments, the active principle of the nucleoside modified messenger RNA (modRNA) drug substance is a single-stranded mRNA that is translated upon entering a cell, e.g., a liver cell. In some embodiments, modRNA contains common structural elements optimized for maximal efficacy of the RNA (5′-cap, 5′-UTR, 3′-UTR, poly(A)-tail). In some embodiments, modRNA contains 1-methyl-pseudouridine instead of uridine. In some embodiments, the 5′-cap structure is m₂^{7,3′-O}Gppp(m₁^{2′-O})ApG. In some embodiments, the 5′-UTR and 3′-UTR comprise the nucleotide sequence of SEQ ID NO: 8 and the nucleotide sequence of SEQ ID NO: 9, respectively. In some embodiments, the poly(A)-tail comprises the sequence of SEQ ID NO: 10. In some embodiments, an additional purification step is applied for modRNA to reduce dsRNA contaminants generated during the in vitro transcription reaction.

Some embodiments of the first RNA and the second RNA are described below. Certain terms used when describing elements thereof have the following meanings:
5′UTR: 5′-UTR sequence of the human alpha-globin mRNA with an optimized ‘Kozak sequence’ to increase translational efficiency.
sec: sec corresponds to a secretory signal peptide (sec), which guides translocation of the nascent polypeptide chain into the endoplasmatic reticulum.
3′UTR: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.
PolyA: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells.
VH(aCD3): Variable region of a heavy chain of an immunoglobulin with specificity for CD3
VL(aCD3): Variable region of a light chain of an immunoglobulin with specificity for CD3
CH1: Constant region 1 of a heavy chain of an immunoglobulin
CL: Constant region of a light chain of an immunoglobulin
VH(aCLDN6): Variable region of a heavy chain of an immunoglobulin with specificity for CLDN6
VL(aCLDN6): Variable region of a light chain of an immunoglobulin with specificity for CLDN6

### RBP022.1 (SEQ ID NO: 4; SEQ ID NO: 5)—Embodiment of “First RNA”

CAP1(m₂^{7,3′-O}Gppp(m₁^{2′-O})ApG)-5′UTR-sec-VH(aCD3)-CH1-VH(aCLDN6)-VL(aCLDN6)-3UTR-PolyA

### RBP021.1 (SEQ ID NO: 6; SEQ ID NO: 7)—Embodiment of “Second RNA”

CAP1(m₂^{7,3′-O}Gppp(m₁^{2′-O})ApG)-5′UTR-sec-VL(aCD3)-CL-VH(aCLDN6)-VL(aCLDN6)-3′UTR-PolyA

### Nucleotide Sequences of RBP022.1 and RBP021.1

Nucleotide sequences are shown with individual sequence elements as indicated in bold letters. In addition, the sequence of the translated protein is shown in italic letters below the coding nucleotide sequence (*=stop codon).

```
RBP022.1
                10 20 30 40 50 53
AGAAYAAACY AGYAYYCYYC YGGYCCCCAC AGACYCAGAG AGAACCCGCC ACC
                                                    hAg-Kozak
                63 73 83 93 103 113
AYGAGAGYGA YGGCCCCYAG AACACYGAYC CYGCYGCYGY CYGGYGCCCY GGCYCYGACA
    M R V M A P R T L I L L L S G A L A L T
                                                    sec
              123 131
GAAACAYGGG CCGGAYCY
    E T W A G S
                sec
              141 151 161 171 181 191
CAGGYGCAGC YCCAGCAAYC YGGYGCCGAA CYYGCYAGAC CYGGCGCCYC CGYGAAGAYG
    Q V Q L Q Q S G A E L A R P G A S V K M
                                                    aCD3 _{VH}
              201 211 221 231 241 251
AGCYGYAAAA CCAGCGGCYA CACCYYCACA CGGYACACCA YGCACYGGGY CAAGCAGAGG
    S C K T S G Y T F T R Y T M H W V K Q R
                                                    aCD3 _{VH}
              261 271 281 291 301 311
CCYGGACAGG GCCYYGAGYG GAYCGGCYAC AYCAACCCCA GCCGGGGCYA CACCAACYAC
    P G Q G L E W I G Y I N P S R G Y T N Y
                                                    aCD3 _{VH}
              321 331 341 351 361 371
AACCAGAAGY YCAAGGACAA GGCCACACYG ACCACCGACA AGAGCAGCAG CACAGCCYAC
    N Q K F K D K A T L T T D K S S S T A Y
                                                    aCD3 _{VH}
              381 391 401 411 421 431
AYGCAGCYGA GCAGCCYGAC CAGCGAAGAY AGCGCCGYGY ACYACYGCGC CCGGYACYAC
    M Q L S S L T S E D S A V Y Y C A R Y Y
                                                    aCD3 _{VH}
              441 451 461 471 481 488
GACGAYCACY ACAGCCYGGA YYACYGGGGC CAGGGAACAA CCCYGACAGY GYCYAGC
    D D H Y S L D Y W G Q G T T L T V S S
                                                    aCD3 _{VH}
              498 508 518 528 538 548
GCCAGCACCA AGGGACCYAG CGYYYYCCCA CYGGCYCCCA GCAGCAAGAG CACAYCYGGY
    A S T K G P S V F P L A P S S K S T S G
                                                    C _{H} 1
              558 568 578 588 598 608
GGAACAGCCG CYCYGGGCYG CCYGGYCAAG GAYYACYYYC CCGAGCCYGY GACCGYGYCC
    G T A A L G C L V K D Y F P E P V T V S
                                                    C _{H} 1
              618 628 638 648 658 668
YGGAAYYCYG GCGCYCYGAC AAGCGGCGYG CACACCYYYC CAGCCGYGCY GCAAAGCAGC
    W N S G A L T S G V H T F P A V L Q S S
                                                    C _{H} 1
              678 688 698 708 718 728
GGCCYGYACY CYCYGAGCAG CGYGGYCACA GYGCCYAGCY CYAGCCYGGG CACCCAGACC
    G L Y S L S S V V T V P S S S L G T Q T
                                                    C _{H} 1
              738 748 758 768 778 782
YACAYCYGCA AYGYGAACCA CAAGCCYAGC AACACCAAGG YGGACAAGAG AGYG
    Y I C N V N H K P S N T K V D K R V
                                                    C _{H} 1
              792 802 812 822 832 842
GAACCCAAGA GCYGYYCYGG ACCCGGCGGA GGAAGAYCYG GCGGAGGCGG YYCYGGYGGC
    E P K S C S G P G G G R S G G G G S G G
                                                    Linker
            851
GGAGGAYCY
    G G S
  Linker
              861 871 881 891 901 911
GAAGYYCAGC YGCAACAGYC YGGCCCCGAG CYGGYYAAGC CYGGGGCCYC YAYGAAGAYC
    E V Q L Q Q S G P E L V K P G A S M K I
                                                    aCLDN6 _{VH}
              921 931 941 951 961 971
YCCYGCAAGG CCYCCGGCYA CAGCYYYACC GGCYACACAA YGAAYYGGGY YAAGCAGYCC
    S C K A S G Y S F T G Y T M N W V K Q S
                                                    aCLDN6 _{VH}
              981 991 1001 1011 1021 1031
CACGGCAAGY GCCYGGAAYG GAYCGGCCYG AYCAACCCYY ACAACGGCGG CACCAYCYAY
    H G K C L E W I G L I N P Y N G G T I Y
                                                    aCLDN6 _{VH}
            1041 1051 1061 1071 1081 1091
AAYCAGAAGY YYAAAGGCAA GGCYACCCYC ACCGYGGACA AGYCYAGCYC CACCGCCYAC
    N Q K F K G K A T L T V D K S S S T A Y
                                                    aCLDN6 _{VH}
            1101 1111 1121 1131 1141 1151
AYGGAACYGC YGAGCCYGAC CYCYGAGGAC YCCGCCGYGY AYYAYYGYGC CAGAGACYAC
    M E L L S L T S E D S A V Y Y C A R D Y
                                                    aCLDN6 _{VH}
            1161 1171 1181 1191 1201 1202
GGCYYCGYGC YGGACYAYYG GGGACAGGGC ACYACACYGA CYGYGYCCAG Y
    G F V L D Y W G Q G T T L T V S S
                                                    aCLDN6 _{VH}
            1212 1222 1232 1242 1252 1262
GGCGGYGGYG GCAGYGGCGG CGGAGGYAGC GGAGGYGGYG GAAGCGGAGG CGGAGGCYCY
    G G G G S G G G G S G G G G S G G G G S
                                                    linker
            1272 1282 1292 1302 1312 1322
CAAAYYGYGC YGACACAGAG CCCCAGCAYC AYGAGCGYYA GCCCYGGCGA GAAAGYGACC
    Q I V L T Q S P S I M S V S P G E K V T
                                                    aCLDN6 _{VL}
            1332 1342 1352 1362 1372 1382
AYCACAYGCA GCGCCAGCYC CYCCGYGYCC YAYAYGCACY GGYYYCAGCA GAAGCCCGGC
    I T C S A S S S VS Y M H W F Q Q K P G
                                                    aCLDN6 _{VL}
            1392 1402 1412 1422 1432 1442
ACAAGCCCCA AGCYGYCGAY CYACAGCACC AGCAACCYGG CCAGCGGAGY GCCYGCCAGA
    T S P K L S I Y S T S N L A S G V P A R
                                                    aCLDN6 _{VL}
            1452 1462 1472 1482 1492 1502
YYYYCYGGYA GAGGCAGCGG CACCAGCYAC YCCCYGACAA YCYCYAGAGY GGCCGCCGAA
    F S G R G S G T S Y S L T I S R V A A E
                                                    aCLDN6 _{VL}
            1512 1522 1532 1542 1552 1562
GAYGCCGCCA CCYACYACYG YCAGCAGCGG AGCAAYYACC CYCCYYGGAC CYYYGGCYGC
    D A A T Y Y C Q Q R S N Y P P W T F G C
                                                    aCLDN6 _{VL}
            1572 1582 1589
GGCACCAAGC YGGAAAYCAA GYGAYGA
    G T K L E I K * *
                          aCLDN6 _{VL}
            1599 1609 1619 1629 1639 1649
GGAYCCGAYC YGGYACYGCA YGCACGCAAY GCYAGCYGCC CCYYYCCCGY CCYGGGYACC
                                                    FI element
            1659 1669 1679 1689 1699 1709
CCGAGYCYCC CCCGACCYCG GGYCCCAGGY AYGCYCCCAC CYCCACCYGC CCCACYCACC
                                                    FI element
            1719 1729 1739 1749 1759 1769
ACCYCYGCYA GYYCCAGACA CCYCCCAAGC ACGCAGCAAY GCAGCYCAAA ACGCYYAGCC
                                                    FI element
            1779 1789 1799 1809 1819 1829
YAGCCACACC CCCACGGGAA ACAGCAGYGA YYAACCYYYA GCAAYAAACG AAAGYYYAAC
                                                    FI element
            1839 1849 1859 1869 1879 1887
YAAGCYAYAC YAACCCCAGG GYYGGYCAAY YYCGYGCCAG CCACACCCYC GAGCYAGC
                                                    FI element
            1897 1907 1917 1927 1937 1947
AAAAAAAAAA AAAAAAAAAA AAAAAAAAAA GCAYAYGACY AAAAAAAAAA AAAAAAAAAA
                                                    Poly(A)
            1957 1967 1977 1987 1997
AAAAAAAAAA AAAAAAAAAA AAAAAAAAAA AAAAAAAAAA AAAAAAAAAA
                                                    Poly(A)
RBP021.1
                10 20 30 40 50 53
AGAAYAAACY AGYAYYCYYC YGGYCCCCAC AGACYCAGAG AGAACCCGCC ACC
                                                    hAg-Kozak
                63 73 83 93 103 113
AYGAGAGYGA YGGCCCCYAG AACACYGAYC CYGCYGCYGY CYGGYGCCCY GGCYCYGACA
    M R M M A P R T L I L L L S G A L A L T
                                                    sec
              123 131
GAAACAYGGG CCGGAYCY
    E T W A G S
                sec
              141 151 161 171 181 191
CAGAYCGYGC YGACACAGAG CCCYGCCAYC AYGAGYGCCY CYCCAGGCGA GAAAGYGACC
    Q I V L T Q S P A I M S A S P G E K V T
                                                    aCD3 _{VL}
              201 211 221 231 241 251
AYGACCYGYA GAGCCAGCAG CAGCGYGYCC YACAYGAACY GGYAYCAGCA GAAGYCCGGC
    M T C R A S S S V S Y M N W Y Q Q K S G
                                                    aCD3 _{VL}
              261 271 281 291 301 311
ACAAGCCCCA AGCGGYGGAY CYACGAYACA AGCAAGGYGG CCAGCGGCGY GCCCYACAGA
    T S P K R W I Y D T S K V A S G V P Y R
                                                    aCD3 _{VL}
              321 331 341 351 361 371
YYYYCYGGCY CYGGCAGCGG CACCAGCYAC AGCCYGACAA YCAGCAGCAY GGAAGCCGAG
    F S G S G S G T S Y S L T I S S M E A E
                                                    aCD3 _{VL}
              381 391 401 411 421 431
GAYGCCGCCA CCYACYACYG CCAGCAGYGG YCCAGCAAYC CCCYGACAYY YGGAGCCGGC
    D A A T Y Y C Q Q W S S N P L T F G A G
                                                    aCD3 _{VL}
              441 449
ACCAAGCYGG AACYGAAG
    T K L E L K
    aCD3 _{VL}
              459 469 479 489 499 509
CGGACAGYYG CCGCYCCYAG CGYGYYCAYC YYCCCACCYY CCGACGAGCA GCYGAAGYCY
    R T V A A P S V F I F P P S D E Q L K S
                                                    C _{L}
              519 529 539 549 559 569
GGAACAGCCA GCGYCGYGYG CCYGCYGAAC AACYYCYACC CYCGGGAAGC CAAGGYGCAG
    G T A S V V C L L N N F Y P R E A K V Q
                                                    C _{L}
              579 589 599 609 619 629
YGGAAGGYGG ACAAYGCCCY CCAGYCCGGC AACAGCCAAG AGAGCGYGAC CGAGCAGGAC
    W K V D N A L Q S G N S Q E S V T E Q D
                                                    C _{L}
              639 649 659 669 679 689
AGCAAGGACY CCACCYAYAG CCYGAGCAGC ACCCYGACAC YGAGCAAGGC CGACYACGAG
    S K D S T Y S L S S T L T L S K A D Y E
                                                    C _{L}
              699 709 719 729 739 749
AAACACAAGG YGYACGCCYG CGAAGYGACC CACCAGGGAC YGYCYAGCCC YGYGACCAAG
    K H K V Y A C E V T H Q G L S S P V T K
                                                    C _{L}
              759 769 770
AGCYYCAACA GAGGCGAGYG Y
    S F N R G E C
              780 788
GAYGYGCCYG GCGGCYCY
    D V P G G S
                Linker
              798 808 818 828 838 848
GAAGYYCAGC YCCAGCAGYC YGGACCCGAG CYGGYYAAGC CYGGCGCCYC CAYGAAGAYC
    E V Q L Q Q S G P E L V K P G A S M K I
                                                    aCLDN6 _{VH}
              858 868 878 888 898 908
YCYYGCAAGG CCYCCGGCYA CAGCYYCACC GGCYACACCA YGAAYYGGGY CAAGCAGAGC
    S C K A S G Y S F T G Y T M N W V K Q S
                                                    aCLDN6 _{VH}
              918 928 938 948 958 968
CACGGCAAGY GCCYGGAAYG GAYCGGCCYG AYCAACCCCY ACAACGGCGG CACAAYCYAC
    H G K C L E W I G L I N P Y N G G T I Y
                                                    aCLDN6 _{VH}
              978 988 998 1008 1018 1028
AACCAGAAGY YCAAGGGCAA AGCCACACYG ACCGYGGACA AGAGCAGCAG CACCGCCYAY
    N Q K F K G K A T L T V D K S S S T A Y
                                                    aCLDN6 _{VH}
            1038 1048 1058 1068 1078 1088
AYGGAACYGC YGAGCCYGAC CAGCGAGGAC YCCGCCGYGY ACYACYGCGC CAGAGAYYAC
    M E L L S L T S E D S A V Y Y C A R D Y
                                                    aCLDN6 _{VH}
            1098 1108 1118 1128 1138 1139
GGCYYCGYGC YGGACYAYYG GGGCCAGGGA ACAACCCYGA CAGYGYCYAG C
    G F V L D Y W G Q G T T L T V S S
                                                    aCLDN6 _{VH}
            1149 1159 1169 1179 1189 1199
GGAGGCGGAG GAYCYGGYGG CGGAGGAAGY GGCGGAGGCG GYYCYGGCGG YGGYGGAYCY
    G G G G S G G G G S G G G G S G G G G S
                                                        linker
            1209 1219 1229 1239 1249 1259
CAAAYYGYCC YGACYCAGYC CCCYAGCAYC AYGAGCGYGY CACCCGGGGA GAAAGYGACA
    Q I V L T Q S P S I M S V S P G E K V T
                                                    aCLDN6 _{VL}
            1269 1279 1289 1299 1309 1319
AYCACCYGYY CCGCCAGCYC CYCCGYGYCC YACAYGCACY GGYYCCAGCA GAAGCCCGGC
    I T C S A S S S V S Y M H W F Q Q K P G
                                                    aCLDN6 _{VL}
            1329 1339 1349 1359 1369 1379
ACCYCCCCCA AGCYGYCCAY CYACYCCACC YCCAACCYGG CCYCCGGCGY GCCCGCCAGA
    T S P K L S I Y S T S N L A S G V P A R
                                                    aCLDN6 _{VL}
            1389 1399 1409 1419 1429 1439
YYCYCYGGCA GAGGCYCCGG CACCAGCYAC YCCCYGACCA YCYCYAGAGY GGCCGCCGAG
    F S G R G S G T S Y S L T I S R V A A E
                                                    aCLDN6 _{VL}
            1449 1459 1469 1479 1489 1499
GACGCYGCCA CAYAYYAYYG YCAGCAGCGG AGCAACYACC CYCCYYGGAC CYYYGGCYGC
    D A A T Y Y C Q Q R S N Y P P W T F G C
                                                    aCLDN6 _{VL}
            1509 1519 1526
GGAACAAAGC YGGAAAYCAA GYGAYGA
    G T K L E I K * *
                        aCLDN6 _{VL}
            1536 1546 1556 1566 1576 1586
GGAYCCGAYC YGGYACYGCA YGCACGCAAY GCYAGCYGCC CCYYYCCCGY CCYGGGYACC
                                                    FI element
            1596 1606 1616 1626 1636 1646
CCGAGYCYCC CCCGACCYCG GGYCCCAGGY AYGCYCCCAC CYCCACCYGC CCCACYCACC
                                                    FI element
            1656 1666 1676 1686 1696 1706
ACCYCYGCYA GYYCCAGACA CCYCCCAAGC ACGCAGCAAY GCAGCYCAAA ACGCYYAGCC
                                                    FI element
            1716 1726 1736 1746 1756 1766
YAGCCACACC CCCACGGGAA ACAGCAGYGA YYAACCYYYA GCAAYAAACG AAAGYYYAAC
                                                    FI element
            1776 1786 1796 1806 1816 1824
YAAGCYAYAC YAACCCCAGG GYYGGYCAAY YYCGYGCCAG CCACACCCYC GAGCYAGC
                                                    FI element
            1834 1844 1854 1864 1874 1884
AAAAAAAAAA AAAAAAAAAA AAAAAAAAAA GCAYAYGACY AAAAAAAAAA AAAAAAAAAA
                                                    Poly(A)
            1894 1904 1914 1924 1934
AAAAAAAAAA AAAAAAAAAA AAAAAAAAAA AAAAAAAAAA AAAAAAAAAA
                                                    Poly(A)
```

In some embodiments, first RNA described herein comprises the nucleotide sequence of SEQ ID NO: 5. In some embodiments, second RNA described herein comprises the nucleotide sequence of SEQ ID NO: 7.

RNA described herein is preferably formulated in lipid nanoparticles (LNP). In some embodiments, the LNP comprise a cationic lipid, a neutral lipid, a steroid, a polymer conjugated lipid; and the RNA. In some embodiments, the cationic lipid is ALC-0366, the neutral lipid is DSPC, the steroid is cholesterol, and the polymer conjugated lipid is ALC-0159. The preferred mode of administration is intravenous administration.

In some embodiments, the drug product comprises the components shown below

| | Molecular | |
|---|---|---|
| Lipid | Weight | Chemical Name and Structure |
| ALC-0366 | 724 Da | ((3-Hydroxypropyl)azanediyl)bis(nonane-9,1-diyl) |
| | | bis(2-butyloctanoate) |
| | | 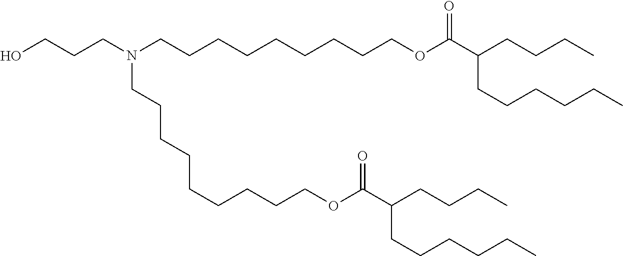 [SMILES: CCCCCCC(CCCC)C(=O)OCCCCCCCCCN(CCCO)CCCCCCCCCOC(=O)C(CCCC)CCCCCC] |
| ALC-0159 | ~2400-2600 Da | 2-[(Polyethylene glycol)-2000]-N,N-ditetradecylacetamide |
| | | 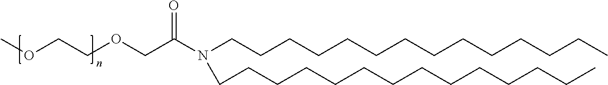 |
| DSPC | 790 Da | 1,2-Distearoyl-sn-glycero-3-phosphocholine |
| | | 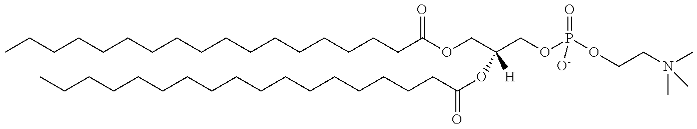 |
| Cho- | 387 Da | Cholest-5-en-3β-ol |
| lesterol | | |
| | | 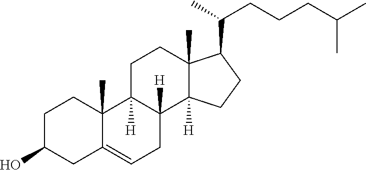 |

In some embodiments, the drug product comprises the components shown below, preferably at the proportions or concentrations shown below:

| | | | Concentration |
|---|---|---|---|
| Component | Quality standard | Function | (mg/ml) |
| BNT142 DS | In-house | Active | 1.00 |
| ALC-0366 | In-house | Functional lipid | 10.64 |
| ALC-0159 | In-house | Functional lipid | 1.35 |
| DSPC | In-house | Structural lipid | 2.43 |
| Cholesterol, | Ph. Eur. | Structural lipid | 4.96 |
| synthetic | | | |
| Sucrose | USP-NF/Ph. Eur. | Cryoprotectant | 102.69 |
| NaCl | USP-NF/Ph. Eur. | Buffer | 6.00 |
| KCl | USP-NF/Ph. Eur. | Buffer | 0.15 |
| Na₂HPO₄ | USP-NF/Ph. Eur. | Buffer | 1.08 |
| KH₂PO₄ | USP-NF/Ph. Eur. | Buffer | 0.15 |
| Water for | Ph. Eur. | Solvent/vehicle | q.s. |
| injection | | | |

ALC-0366, ((3-hydroxypropyl)azanediyl)bis(nonane-9,1-diyl) bis(2-butyloctanoate);
ALC-0159, 2-[(polyethylene glycol)-2000]-N,N-ditetradecylacetamide;
DSPC = 1,2-distearoyl-sn-glycero-3-phosphocholine;
DS = drug substance;
Ph. Eur. = Pharmacopoea Europaea;
q.s. = quantum satis (as much as may suffice);
USP-NF = United States Pharmacopeia and National Formulary.

In some embodiments, the ratio of mRNA to total lipid (N/P) is between 6.0 and 6.5 such as about 6.0 or about 6.3.

### Pharmaceutical Compositions

The agents described herein may be administered in pharmaceutical compositions or medicaments and may be administered in the form of any suitable pharmaceutical composition.

In some embodiments where a pharmaceutical composition comprises a first RNA and a second RNA as described herein, such first RNA and second RNA may be present in a molar ratio of about 3:1 to about 1:3, or in some embodiments in a molar ratio of about 2:1 to about 1:2, or in some embodiments in a molar ratio of about 1.5:1 to about 1:1.5. In some embodiments, such first RNA and second RNA may be present in a molar ratio of about 3:1 to about 1:1, or in some embodiments in a molar ratio of about 2:1 to about 1:1, or in some embodiments in a molar ratio of about 1.5:1.

In some embodiments where a pharmaceutical composition comprises a first RNA and a second RNA as described herein, such first RNA and second RNA may be present in a weight (w/w) ratio of about 3:1 to about 1:3, or in some embodiments in a (w/w) ratio of about 2:1 to about 1:2, or in some embodiments in a (w/w) ratio of about 1.5:1 to about 1:1.5. In some embodiments, such first RNA and second RNA may be present in a (w/w) ratio of about 3:1 to about 1:1, or in some embodiments in a (w/w) ratio of about 2:1 to about 1:1, or in some embodiments in a (w/w) ratio of about 1.75:1 to about 1.25:1, or in some embodiments in a (w/w) ratio of about 1.75:1 to about 1.5:1, or in some embodiments in a (w/w) ratio of about 1.5:1 to about 1.25:1, or in some preferred embodiments in a (w/w) ratio of about 1.5:1.

In some embodiments, the pharmaceutical composition described herein is a composition for treating cancer in a subject.

In some embodiments of all aspects of the invention, the components described herein such as RNA encoding a binding agent may be administered in a pharmaceutical composition which may comprise a pharmaceutically acceptable carrier and may optionally comprise one or more adjuvants, stabilizers etc. In some embodiments, the pharmaceutical composition is for therapeutic or prophylactic treatments, e.g., for use in treating or preventing cancer.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

In some embodiments, each dose or a cumulative dose of a pharmaceutical composition described herein may comprise RNA encoding a CLDN6-targeting binding agent in an amount of 0.05 ag/kg or more, e.g., in an amount within a range of 0.05 ag/kg to 5 mg/kg, or 0.05 ag/kg to 500 ag/kg, or 0.5 ag/kg to 500 ag/kg, or 1 ag/kg to 50 ag/kg, or 5 ag/kg to 150 ag/kg, or 15 ag/kg to 150 ag/kg, wherein kg refers to kg body weight of a subject to be treated. As will be clear to the skilled person, since the binding agents disclosed herein comprise two polypeptides each encoded by a separate RNA, the indicated amounts relate to the cumulative amount of RNA encoding the first and second polypetide chain. Preferably each dose or cumulative dose comprises a mixture of RNA encoding the first and the second polypetide chains in a total amount of 5 ag/kg to 150 ag/kg, or 15 ag/kg to 150 ag/kg.

In some embodiments, a pharmaceutical composition described herein is administered to deliver RNA described herein (e.g., mRNA) encoding a binding agent directed to CLDN6 to achieve a level (e.g., plasma level and/or tissue level) of binding agent of about 0.05 ng/mL or more.

In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

In some embodiments, a pharmaceutical composition described herein is administered to a subject suffering from a CLDN6-positive cancer, e.g., a CLDN6-positive solid tumor, in at least one or more (including, e.g., at least two, at least three, at least four, at least five, at least six, at least seven, at least eight, or more) dosing cycles. In some embodiments, each dosing cycle may be a three-week dosing cycle. In some embodiments, a pharmaceutical composition described herein is administered in at least one dose per dosing cycle. In some embodiments, a dosing cycle involves administration of a set number and/or pattern of doses; in some embodiments, a dosing cycle involves administration of a set cumulative dose, e.g., over a particular period of time, and optionally via multiple doses, which may be administered, for example, at set interval(s) and/or according to a set pattern.

Those skilled in the art are aware that cancer therapeutics often administered in dosing cycles. In some embodiments, pharmaceutical compositions described herein are administered in one or more dosing cycles.

In some embodiments, one dosing cycle is at least 3 or more days (including, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 days.

In some embodiments, one dosing cycle may involve multiple doses, e.g., according to a pattern such as, for example, a dose may be administered daily within a cycle, or a dose may be administered every 2 days, every 3 days, every 4 days, every 5 days, every 6 days, every 7 days within a cycle.

In some embodiments, multiple cycles may be administered. For example, in some embodiments, at least 2 cycles (including, e.g., at least 3 cycles, at least 4 cycles, at least 5 cycles, at least 6 cycles, at least 7 cycles, at least 8 cycles, at least 9 cycles, at least 10 cycles, or more) can be administered. In some embodiments, at least 3-8 dosing cycles may be administered.

In some embodiments, there may be a “rest period” between cycles; in some embodiments, there may be no rest period between cycles. In some embodiments, there may be sometimes a rest period and sometimes no rest period between cycles.

The pharmaceutical compositions of the present disclosure may contain salts, buffers, preservatives, and optionally other therapeutic agents. In some embodiments, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In some embodiments, a pharmaceutical composition described herein may further comprise one or more additives, for example, in some embodiments that may enhance stability of such a composition under certain conditions. For example, in some embodiments, a pharmaceutical composition may further comprise a cryoprotectant (e.g., sucrose) and/or an aqueous buffered solution, which may in some embodiments include one or more salts (e.g., sodium salts). In some embodiments, pharmaceutical compositions described herein may be administered intravenously, intraarterially, subcutaneously, intradermally or intramuscularly. In some embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In some embodiments, the pharmaceutical composition is formulated for systemic administration, e.g., for intravenous administration.

The term “co-administering” as used herein means a process whereby different compounds or compositions are administered to the same patient. The different compounds or compositions may be administered simultaneously, at essentially the same time, or sequentially.

### Effects of the Agents and Treatments Described Herein

The treatment described herein may result in immune effector functions, e.g., T cell-mediated effector functions, on target cells which may result in the killing of target cells. In some embodiments, the effector functions in the context of the present invention comprise the activation of cytotoxic CD4+ and/or CD8+ lymphocytes (CTLs) and the elimination of target cells, i.e., cells characterized by expression of antigen, i.e., CLDN6, for example, via apoptosis or perforin-mediated apoptosis and cell lysis, production of cytokines such as IFN-γ and TNF-α, and specific cytotoxic of antigen-expressing target cells.

Upon activation, cytotoxic lymphocytes may trigger the destruction of target cells. For example, cytotoxic T cells may trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells.

In some embodiments, binding agents described herein that comprises two binding domains that are specific for CLDN6 expressed by cancer cells and a binding domain that is specific for CD3 expressed by T cells, target the cytotoxic effect of T cells expressing CD3 to cancer cells expressing CLDN6. In some embodiments, binding of the binding agent to CD3 on T cells results in proliferation and/or activation of the T cells. In some embodiments, the T cells release cytotoxic factors, e.g. perforins and granzymes, and initiate cytolysis and apoptosis of cancer cells. In some embodiments, the binding agent induces T cell-mediated cytotoxicity against cancer cells expressing CLDN6. In some embodiments, the binding agent elicits immune effector functions as described herein. In some embodiments, said immune effector functions are directed against cells carrying the tumor-associated antigen CLDN6 on their surface.

In some embodiments, the cancer cell(s) is/are from a cancer selected from the group consisting of urinary bladder cancer, ovarian cancer, in particular ovarian adenocarcinoma and ovarian teratocarcinoma, lung cancer, including small cell lung cancer (SCLC) and non-small cell lung cancer (NSCLC), in particular squamous cell lung carcinoma and adenocarcinoma or non-small cell lung cancer (NSCLC) of non-squamous type, gastric cancer, breast cancer, hepatic cancer, pancreatic cancer, skin cancer, in particular basal cell carcinoma and squamous cell carcinoma, malignant melanoma, head and neck cancer, in particular malignant pleomorphic adenoma, sarcoma, in particular synovial sarcoma and carcinosarcoma, bile duct cancer, cancer of the urinary bladder, in particular transitional cell carcinoma and papillary carcinoma, kidney cancer, in particular renal cell carcinoma including clear cell renal cell carcinoma and papillary renal cell carcinoma, colon cancer, small bowel cancer, including cancer of the ileum, in particular small bowel adenocarcinoma and adenocarcinoma of the ileum, testicular embryonal carcinoma, placental choriocarcinoma, cervical cancer, testicular cancer, in particular testicular seminoma, testicular teratoma and embryonic testicular cancer, uterine cancer, germ cell tumors such as a teratocarcinoma or an embryonal carcinoma, in particular germ cell tumors of the testis, and the metastatic forms thereof.

As used herein, “immune response” refers to an integrated bodily response to an antigen or a cell expressing an antigen and refers to a cellular immune response and/or a humoral immune response. The immune system is divided into the first line innate immune system, and acquired or adaptive immune system of vertebrates, each of which contains humoral and cellular components.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to immune effector cells, in particular to cells called T cells or T lymphocytes which act as either “helpers” or “killers”. The helper T cells (also termed CD4+ T cells) play a central role by regulating the immune response and the killer cells (also termed cytotoxic T cells, cytolytic T cells) kill diseased cells such as virus-infected cells, preventing the production of more diseased cells.

The term “immune effector cell” or “immunoreactive cell” in the context of the present invention relates to a cell which exerts effector functions during an immune reaction. For example, immune effector cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4+ and/or CD8+ T cells, most preferably CD8+ T cells. According to the invention, the term “immune effector cell” also includes a cell which can mature into an immune cell (such as T cell, in particular T helper cell, or cytolytic T cell) with suitable stimulation. Immune effector cells comprise CD34+ hematopoietic stem cells, immature and mature T cells and immature and mature B cells. The differentiation of T cell precursors into a cytolytic T cell, when exposed to an antigen, is similar to clonal selection of the immune system.

The terms “T cell” and “T lymphocyte” are used interchangeably herein and include but are not limited to T helper cells (CD4+ T cells) and cytotoxic T cells (CTLs) which comprise cytolytic T cells. T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptor (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4+ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8+ T cells since they express the CD8 glycoprotein on their surface. These cells recognize their targets by binding to antigen associated with MHC class I, which is present on the surface of nearly every cell of the body.

A majority of T cells have a T cell receptor (TCR) existing as a complex of several proteins. The TCR of a T cell is able to interact with immunogenic peptides (epitopes) bound to major histocompatibility complex (MHC) molecules and presented on the surface of target cells. Specific binding of the TCR triggers a signal cascade inside the T cell leading to proliferation and differentiation into a maturated effector T cell. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

“Humoral immunity” or “humoral immune response” is the aspect of immunity that is mediated by macromolecules found in extracellular fluids such as secreted antibodies, complement proteins, and certain antimicrobial peptides. It contrasts with cell-mediated immunity. Its aspects involving antibodies are often called antibody-mediated immunity.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In some embodiments, the macrophages are splenic macrophages.

The term “dendritic cell” (DC) refers to another subtype of phagocytic cells belonging to the class of antigen presenting cells. In some embodiments, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In some embodiments, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division. The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which immune effector cells are stimulated, proliferate, and the specific immune effector cell is amplified. Preferably, clonal expansion leads to differentiation of the immune effector cells.

### Treatments

The present invention provides methods and agents for treating or preventing diseases or disorders as described herein, in particular, diseases associated with expression of CLDN6, such CLDN6-positive cancer, in a subject. Methods described herein may comprise administering an effective amount of a composition comprising RNA encoding a binding agent described herein.

The term “disease associated with expression” when it refers to an antigen refers to any disease which implicates the antigen, e.g. a disease which is characterized by the presence of the antigen. The antigen may be a disease-associated antigen, such as a tumor-associated antigen, e.g., CLDN6. In some embodiments, a disease associated with expression of an antigen is a disease involving cells expressing an antigen, preferably on the cell surface.

The therapeutic compounds or compositions of the invention may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In some embodiments, administration of an agent or composition of the present invention may be performed by single administration or boosted by multiple administrations.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

As used herein, the term “disease” includes cancer, in particular those forms of cancer described herein. Any reference herein to cancer or particular forms of cancer also includes cancer metastasis thereof. In some embodiments, a disease to be treated according to the present application is associated with cells expressing CLDN6.

“Diseases associated with cells expressing CLDN6” or similar expressions means according to the invention that CLDN6 is expressed in cells of a diseased tissue or organ. In some embodiments, expression of CLDN6 in cells of a diseased tissue or organ is increased compared to the state in a healthy tissue or organ. An increase refers to an increase by at least 10%, in particular at least 20%, at least 50%, at least 100%, at least 200%, at least 500%, at least 1,000%, at least 10,000% or even more. In some embodiments, expression is only found in a diseased tissue, while expression in a healthy tissue is repressed. According to the invention, diseases associated with cells expressing CLDN6 include cancer diseases. Furthermore, according to the invention, cancer diseases preferably are those wherein the cancer cells express CLDN6.

As used herein, a “cancer disease” or “cancer” includes a disease characterized by aberrantly regulated cellular growth, proliferation, differentiation, adhesion, and/or migration. By “cancer cell” is meant an abnormal cell that grows by a rapid, uncontrolled cellular proliferation and continues to grow after the stimuli that initiated the new growth cease. Preferably, a “cancer disease” is characterized by cells expressing CLDN6 and a cancer cell expresses CLDN6. A cell expressing CLDN6 preferably is a cancer cell, preferably of the cancers described herein.

The term “cancer” according to the invention comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. Examples thereof are lung carcinomas, mamma carcinomas, prostate carcinomas, colon carcinomas, renal cell carcinomas, cervical carcinomas, or metastases of the cancer types or tumors described above. The term cancer according to the invention also comprises cancer metastases.

According to the invention, a “carcinoma” is a malignant tumor derived from epithelial cells. This group represents the most common cancers, including the common forms of breast, prostate, lung and colon cancer.

“Adenocarcinoma” is a cancer that originates in glandular tissue. This tissue is also part of a larger tissue category known as epithelial tissue. Epithelial tissue includes skin, glands and a variety of other tissue that lines the cavities and organs of the body. Epithelium is derived embryologically from ectoderm, endoderm and mesoderm. To be classified as adenocarcinoma, the cells do not necessarily need to be part of a gland, as long as they have secretory properties. This form of carcinoma can occur in some higher mammals, including humans. Well differentiated adenocarcinomas tend to resemble the glandular tissue that they are derived from, while poorly differentiated may not. By staining the cells from a biopsy, a pathologist will determine whether the tumor is an adenocarcinoma or some other type of cancer. Adenocarcinomas can arise in many tissues of the body due to the ubiquitous nature of glands within the body. While each gland may not be secreting the same substance, as long as there is an exocrine function to the cell, it is considered glandular and its malignant form is therefore named adenocarcinoma. Malignant adenocarcinomas invade other tissues and often metastasize given enough time to do so. Ovarian adenocarcinoma is the most common type of ovarian carcinoma. It includes the serous and mucinous adenocarcinomas, the clear cell adenocarcinoma and the endometrioid adenocarcinoma.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential. In some embodiments, the term “metastasis” according to the invention relates to “distant metastasis” which relates to a metastasis which is remote from the primary tumor and the regional lymph node system. In some embodiments, the term “metastasis” according to the invention relates to lymph node metastasis. One particular form of metastasis which is treatable using the therapy of the invention is metastasis originating from gastric cancer as primary site. In preferred embodiments such gastric cancer metastasis is Krukenberg tumors, peritoneal metastasis and/or lymph node metastasis.

As used herein, the term “unresectable tumor” typically refers to a tumor characterized by one or more features that, in accordance with sound medical judgement, are considered to indicate that the tumor cannot safely (e.g., without undue harm to the subject) be removed by surgery, and/or with respect to which a competent medical profession has determined that risk to the subject of tumor removal outweighs benefits associated with such removal. In some embodiments, an unresectable tumor refers to a tumor that involves and/or has grown into an essential organ or tissue (including blood vessels that may not be reconstructable) and/or that is otherwise in a location that cannot readily be surgically accessed without unreasonable risk of damage to one or more other critical or essential organs and/or tissues (including blood vessels). In some embodiments, “unresectability” of a tumor refers to the likelihood of achieving a margin-negative (R0) resection. In the context of pancreatic cancer, encasement of major vessels by a tumor such as superior mesenteric artery (SMA) or celiac axis, portal vein occlusion, and the presence of celiac or para-aortic lymphadenopathy are generally acknowledged as findings that preclude R0 surgery. Those skilled in the art will understand parameters that determine whether a tumor is unresectable or not.

“Target cell” shall mean any undesirable cell such as a cancer cell. In preferred embodiments, the target cell expresses CLDN6.

In some embodiments, a CLDN6-positive cancer comprises a CLDN6-positive advanced solid tumor. In some embodiments, a CLDN6-positive cancer is selected from the group consisting of advanced/metastatic CLDN6-positive ovarian cancer, non-small cell lung cancer (NSCLC) of non-squamous type, endometrial and testicular cancer, in particular for whom there is no available standard therapy likely to confer clinical benefit. In some embodiments, a CLDN6-positive cancer includes Not Otherwise Specified (NOS) tumors including rare tumors and cancers of unknown primary. Such cancers can be tested for CLDN6 expression.

In some embodiments, a CLDN6-positive cancer is selected from the group consisting of urinary bladder cancer, ovarian cancer, in particular ovarian adenocarcinoma and ovarian teratocarcinoma, lung cancer, including small cell lung cancer (SCLC) and non-small cell lung cancer (NSCLC), in particular squamous cell lung carcinoma and adenocarcinoma or non-small cell lung cancer (NSCLC) of non-squamous type, gastric cancer, breast cancer, hepatic cancer, pancreatic cancer, skin cancer, in particular basal cell carcinoma and squamous cell carcinoma, malignant melanoma, head and neck cancer, in particular malignant pleomorphic adenoma, sarcoma, in particular synovial sarcoma and carcinosarcoma, bile duct cancer, cancer of the urinary bladder, in particular transitional cell carcinoma and papillary carcinoma, kidney cancer, in particular renal cell carcinoma including clear cell renal cell carcinoma and papillary renal cell carcinoma, colon cancer, small bowel cancer, including cancer of the ileum, in particular small bowel adenocarcinoma and adenocarcinoma of the ileum, testicular embryonal carcinoma, placental choriocarcinoma, cervical cancer, testicular cancer, in particular testicular seminoma, testicular teratoma and embryonic testicular cancer, uterine cancer, germ cell tumors such as a teratocarcinoma or an embryonal carcinoma, in particular germ cell tumors of the testis, and the metastatic forms thereof.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder but mayor may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Physical, Chemical and Pharmaceutical Properties of BNT142

### Drug Substance

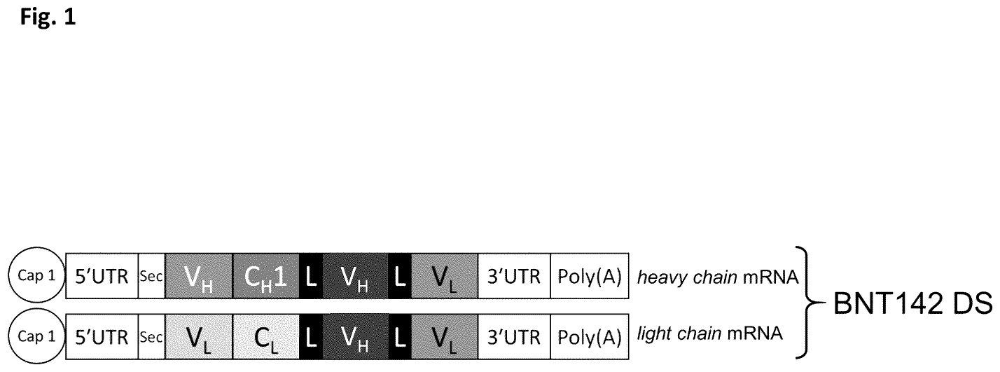

BNT142 drug substance is an RNA mixture coding for the heavy chain (HC) and the light chain (LC) of the antigen-binding fragment (Fab)-(scFv)ᵣ-based bispecific antibody—or tribody—directed against CLDN6 and CD3, henceforth referred to as RiboMab02.1. RiboMab02.1 recognizes conformational epitopes of CLDN6 and the CD3 epsilon (E) chain. The active ingredients of the drug substance are a mixture of two single-stranded, 5′-capped, and N1-methylpseudouridine modified RNAs that are translated into the respective protein subunits and form the full bispecific antibody in targeted cells. FIG. 1 shows a schematic illustration of the general structure of the protein-encoding RNAs, which are determined by the respective nucleotide sequence of the linearized plasmid deoxyribonucleic acid (DNA) used as a template for in vitro RNA transcription. In addition to the sequence encoding the HC and the LC, each RNA contains common structural elements optimized for maximal efficacy with respect to stability and translational efficiency (5′-cap, 5′-untranslated regions [UTR], 3′-UTR, and poly[A]-tail).

### Drug Product

### Description of the Drug Product

The drug product is a sterile RNA-LNP dispersion in aqueous buffer for IV administration. The drug product manufacturing involves the encapsulation of RNA in nano-scale lipid particles comprised of specialized lipid components. Once formulated in an LNP, the RNA drug substance is protected from degradation in the serum. The LNP formulation enables escape of the RNA from the endosomal compartment into the cell cytosol where it is available for translation into functional protein. The composition of the BNT142 drug product is given in Table 2.

TABLE 2
Composition of BNT142 drug product

| | | | Concentration |
|---|---|---|---|
| Component | Quality standard | Function | (mg/ml) |
| BNT142 DS | In-house | Active | 1.0 |
| ALC-0366 | In-house | Functional lipid | 10.64 |
| ALC-0159 | In-house | Functional lipid | 1.35 |
| DSPC | In-house | Structural lipid | 2.43 |
| Cholesterol, | Ph. Eur. | Structural lipid | 4.96 |
| synthetic | | | |
| Sucrose | USP-NF/Ph. Eur. | Cryoprotectant | 102.69 |
| NaCl | USP-NF/Ph. Eur. | Buffer | 6.00 |
| KCl | USP-NF/Ph. Eur. | Buffer | 0.15 |
| Na₂HPO₄ | USP-NF/Ph. Eur. | Buffer | 1.08 |
| KH₂PO₄ | USP-NF/Ph. Eur. | Buffer | 0.15 |
| Water | for | Solvent/vehicle | q.s. |
| injection | Ph. Eur. | | |

ALC-0366, ((3-hydroxypropyl)azanediyl)bis(nonane-9,1-diyl) bis(2-butyloctanoate);
ALC-0159, 2-[(polyethylene glycol)-2000]-N,N-ditetradecylacetamide;
DSPC = 1,2-distearoyl-sn-glycero-3-phosphocholine;
DS = drug substance;
Ph. Eur. = Pharmacopoea Europaea;
q.s. = quantum satis (as much as may suffice);
USP-NF = United States Pharmacopeia and National Formulary.

The drug product pH and osmolality typically range from pH 6.9 to 7.9 and from 425 to 625 mOsmol/kg, respectively. The LNP particle size, measured by dynamic light scattering (DLS), ranges from approximately 50 to 100 nm with a polydispersity index of less than or equal to 0.2. The particle size for the drug product has been selected to optimize its ability to reach the target tissue, its drug loading capacity, and product safety.

Upon uptake by targeted cells, RNAs encoding the heavy and the light chain are translated into the respective proteins, folded, and assembled forming the full bispecific antibody, which is subsequently secreted. The antibody is systemically available to act specifically on tumor cells expressing CLDN6 by recruiting cytotoxic T cells to tumor cells and induce target-dependent polyclonal T-cell activation and tumor cell lysis.

The BNT142 drug product is provided as a frozen concentrated suspension for injection in single use glass vials containing 1 mg/mL of RNA (comprising both the first and second RNA at a weight ratio of 1.5:1) formulated as RNA-LNP. After thawing, the drug product is diluted in commercially available isotonic NaCl solution (0.9%) and filtered with an in-line filter. The IMP is administered to patients by IV administration.

### Example 2: Non-Clinical Studies

The BNT142 non-clinical package comprises primary PD studies, establishing proof-of-concept and assessing the mode-of-action of RiboMab02.1, as well as secondary PD studies, predicting off-target profiling and investigating non-specific T-cell activation and cytokine release by RiboMab02.1. Furthermore, tissue cross-reactivity of the CLDN6-binding part of RiboMab02.1 was studied in a GLP-compliant study. Although BNT142 technically does not fall within their scope, International Council for Harmonization (ICH) guidelines S6, S7A/B, and S9, as well as FDA recommendations for CD3 binding T-cell engagers were followed.

The above guidelines were also considered when assessing safety aspects of the drug product (RNA-LNP). Here, a platform approach has been used in safety pharmacology and (immuno-)toxicity studies. As demonstrated before, safety aspects intrinsic to the RNA platform and used LNPs are largely independent of the RNA sequence or length, but rather depend on the dose and type of RNA and/or LNP. For this reason, a single-dose and a GLP-compliant repeated-dose toxicity study including safety pharmacology readouts were performed as an RNA-LNP platform study. In addition to including immunotoxicological readouts in these in vivo studies, drug product-mediated immunotoxicity was also addressed in vitro.

The PK studies address the different facets of the product. Firstly, the PK profile of the encoded RiboMab02.1 was characterized in different species, elucidating the dose exposure of BNT142. Secondly, platform properties such as the biodistribution of the LNP and protein expression were addressed, using surrogate RNA. In addition, the metabolism of ALC-0366 and ALC-0159 was evaluated in vitro and for ALC-0159 also in vivo in rat plasma, urine, feces, and liver samples from a study, which also characterized the plasma and liver PK profile of this lipid.

### 2.1 Non-Clinical Pharmacology

### Primary Pharmacodynamics

Primary PD studies are those in vitro and in vivo studies that investigate effects such as the translation of BNT142 drug substance into protein and the desired mode-of-action of the encoded bispecific protein RiboMab02.1 in relation to its therapeutic targets human CD3 and CLDN6.

### RiboMab02.1 Binds Specifically to Human CD3 and the Oncofetal Antigen CLDN6

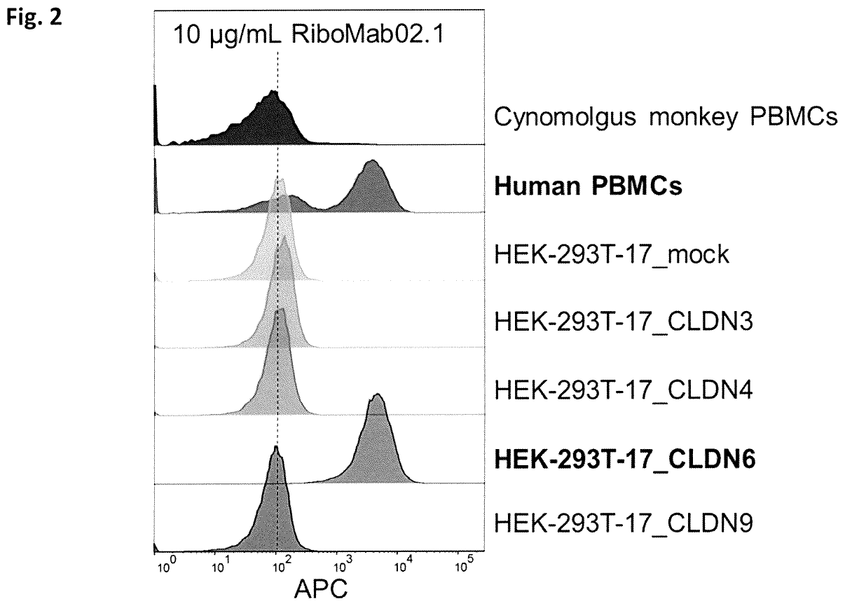

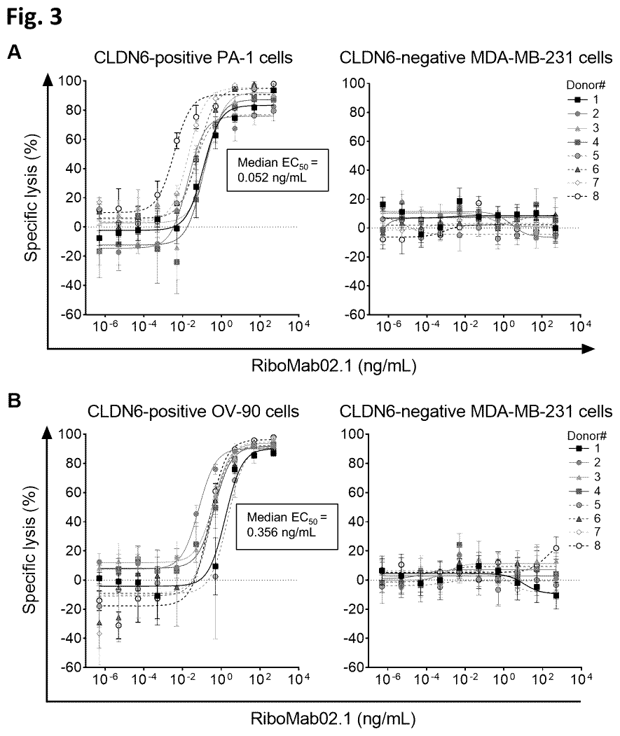

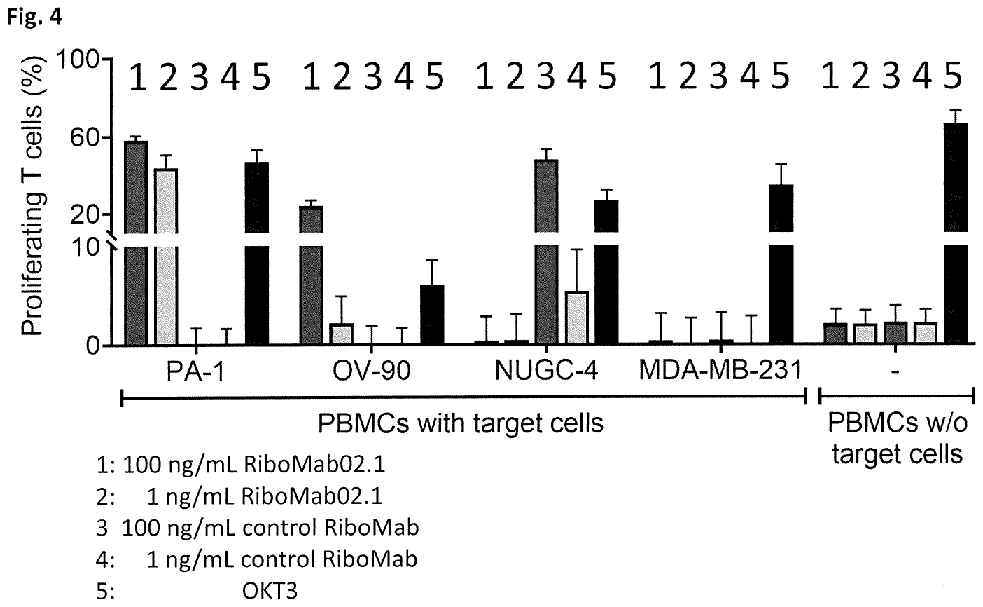

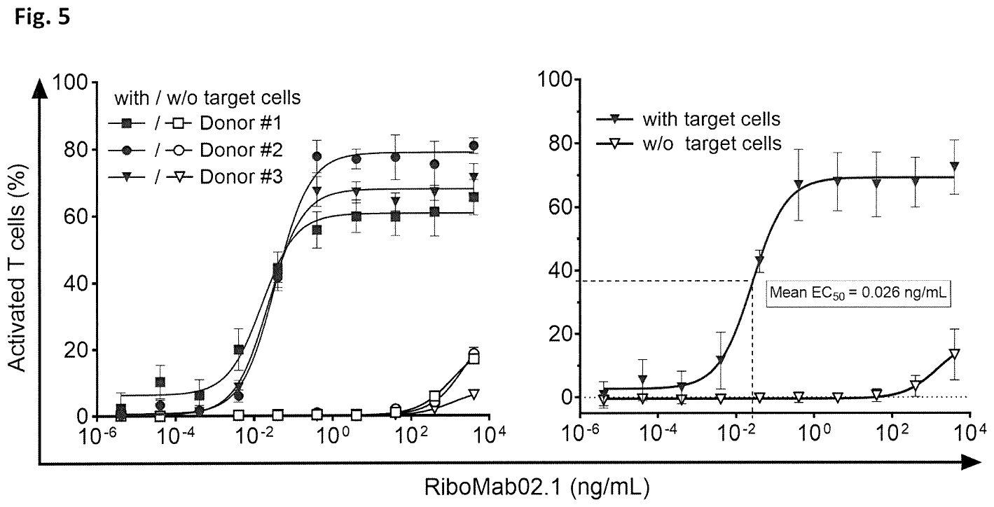

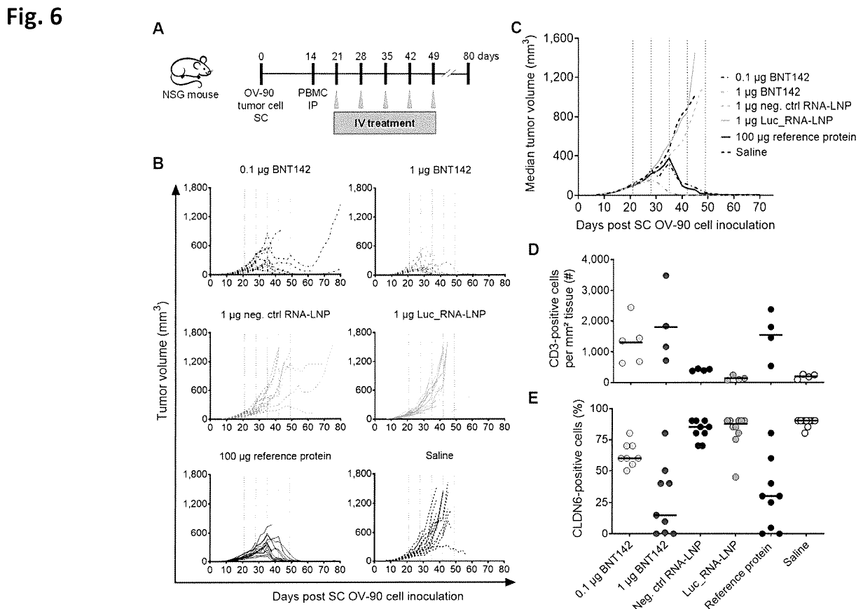

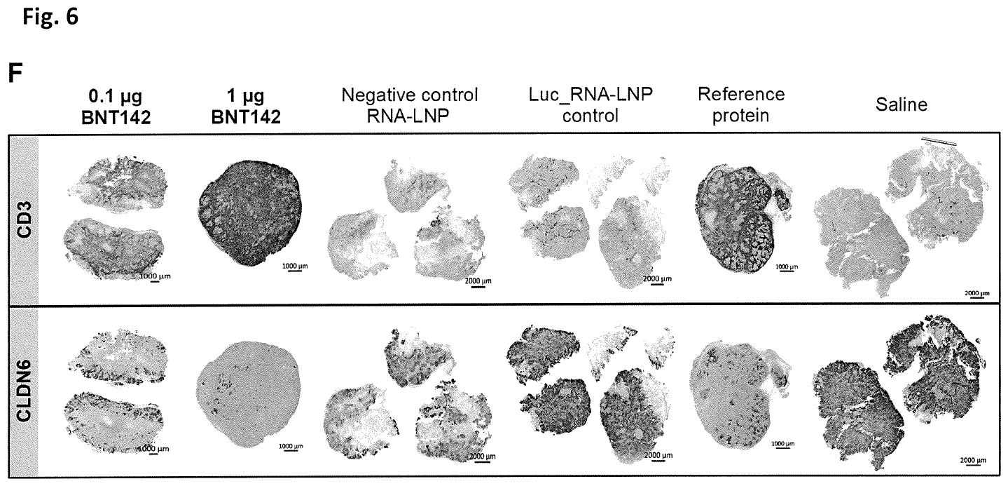

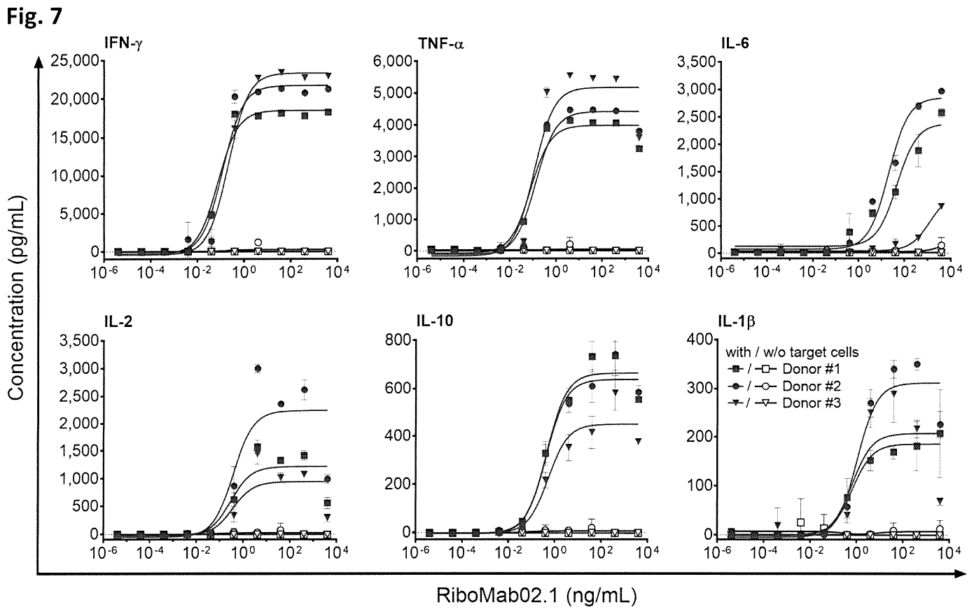

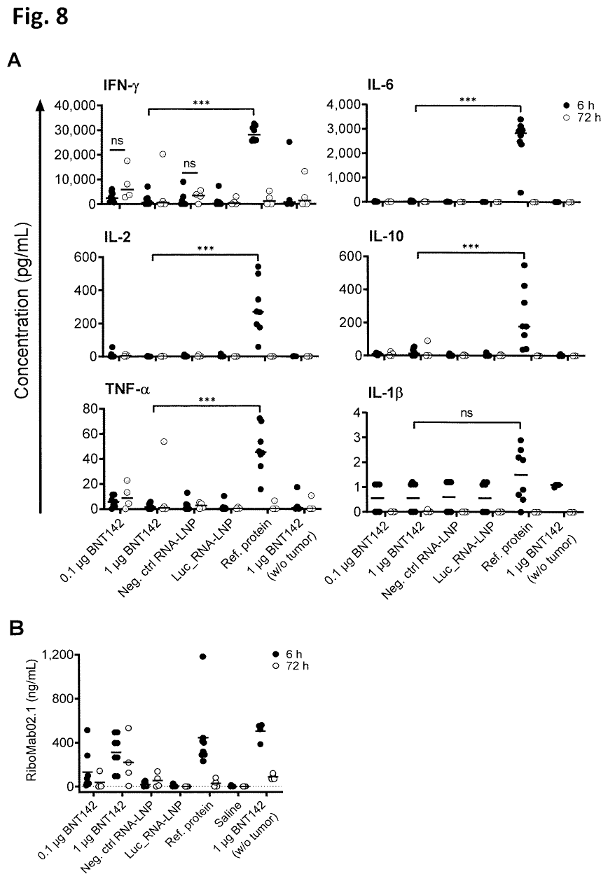

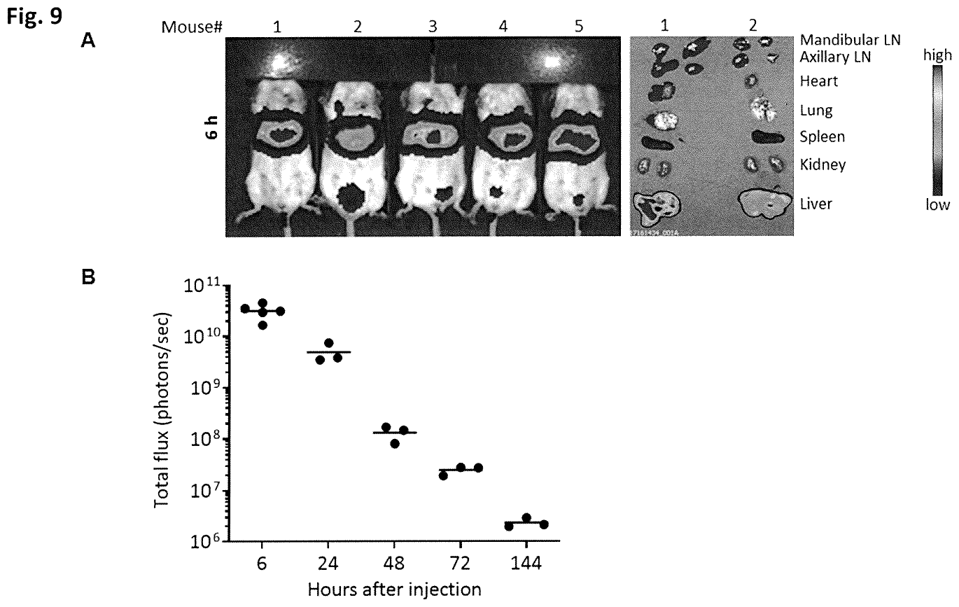

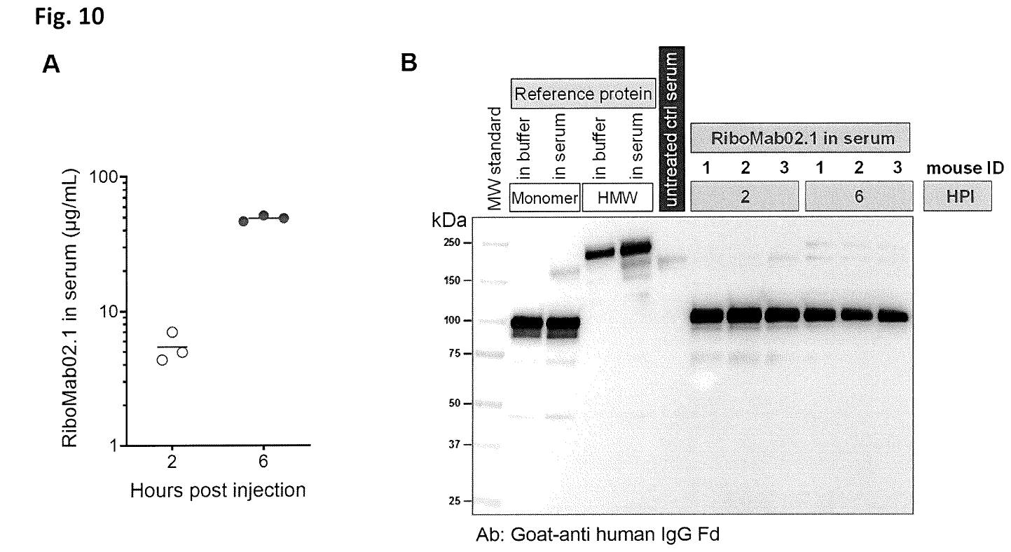

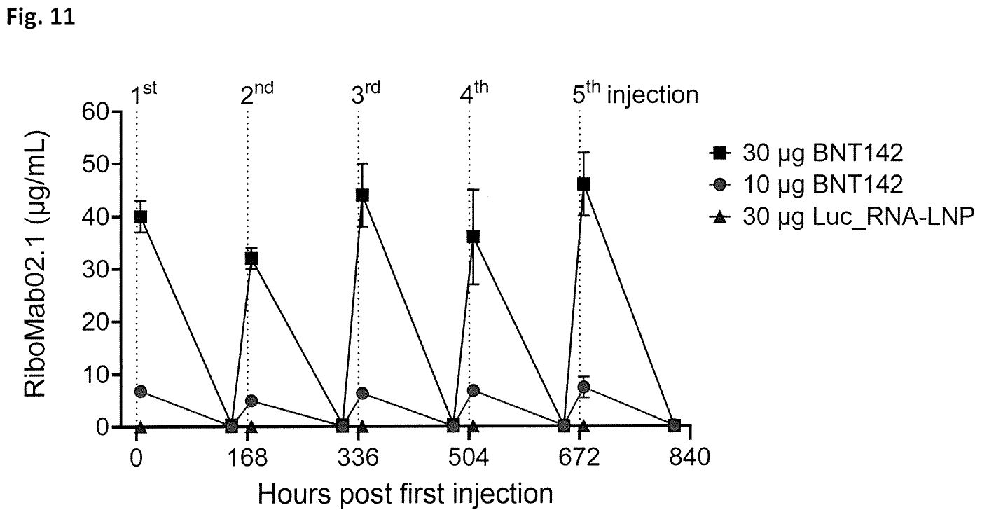

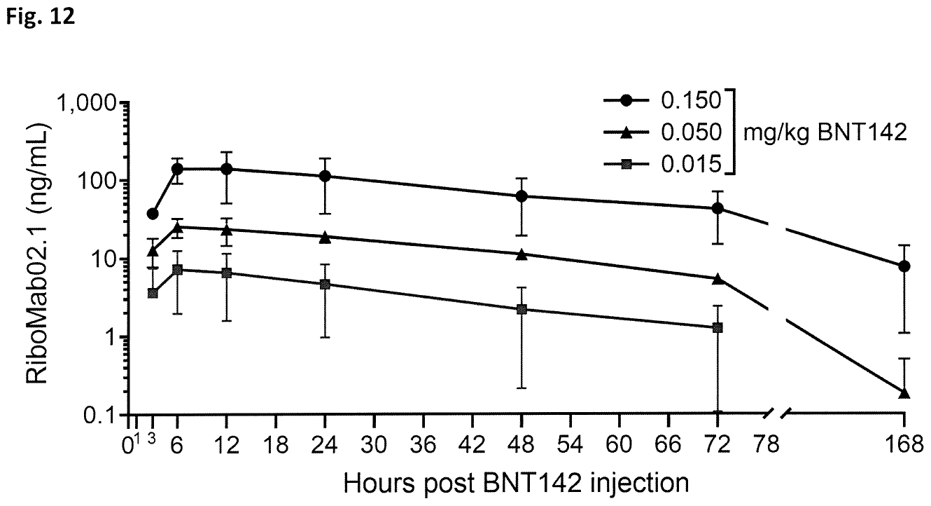

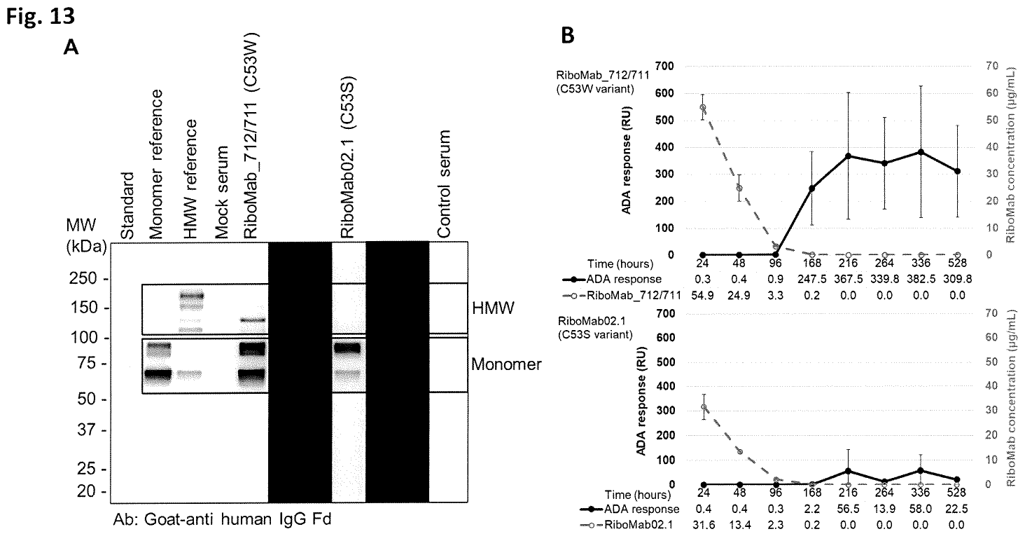

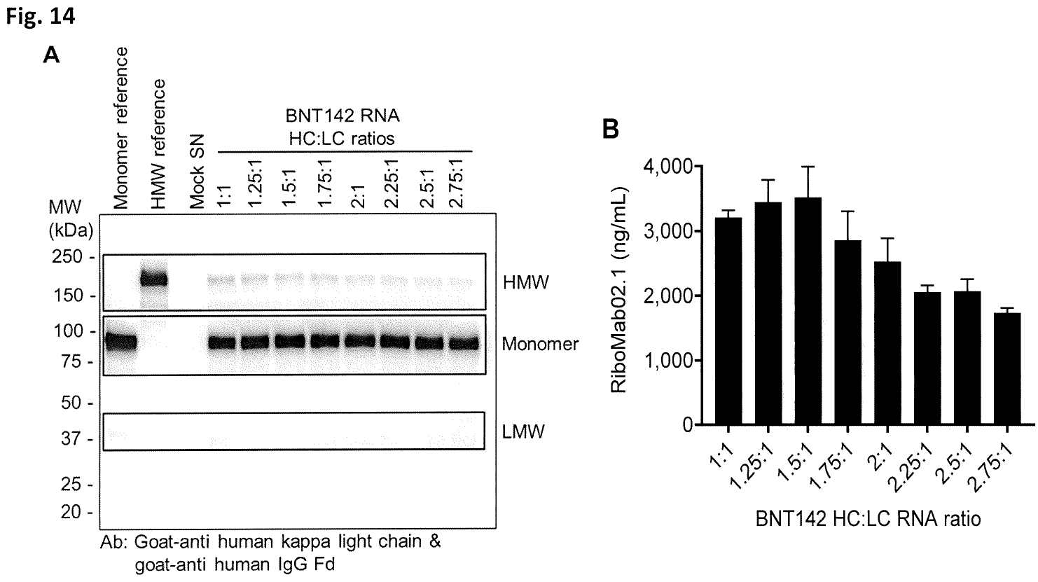

To determine the target specificity of RiboMab02.1 for CLDN6 and human CD3, flow cytometric binding assays were conducted using cell culture supernatant containing RiboMab02.1 expressed in HEK-293T-17 producer cells transfected with BNT142. Human peripheral blood mononuclear cells (PBMCs) served as target cells for the RiboMab02.1 CD3-binding arm while cynomolgus monkey-derived PBMCs served as species control. HEK-293T-17 cells transduced with CLDN6 for stable protein expression were used as target cells for the two RiboMab02.1 CLDN6-binding arms. To assess cross-reactivity of RiboMab02.1 to the closely related claudins CLDN3, CLDN4 and CLDN9, binding of RiboMab02.1 to HEK-293T-17 stable transductants, each heterologously expressing one of the CLDN molecules, was tested. RiboMab02.1 bound specifically to CLDN6 and not to CLDN3, CLDN4 or CLDN9. Similarly, only human but not simian CD3 was recognized by RiboMab02.1 (FIG. 2).

### In Vivo-Produced RiboMab02.1 Mediates Dose-Dependent and Target-Specific Tumor Cell Lysis

The bioactivity of RiboMab02.1, produced in BNT142-administered Balb/cJRj mice, was assessed ex vivo by analyzing the cytotoxic potential in T-cell-mediated cytotoxicity assays. Bioluminescence-based cytotoxicity assays were conducted using luciferase-expressing cell lines.

The CLDN6-positive human ovarian cancer cell lines PA-1 and OV-90 served to determine target-dependent lysis efficiency while the target-negative human breast cancer cell line MDA-MB-231 was used as negative control to confirm target-specific lysis. Human PBMCs from eight different healthy donors were used as effector cells to reflect the donor-dependent heterogeneity of human T-cell responses. In accordance with the doubling times of the target-positive cell lines, PA-1-containing assays were incubated for 24 h and OV-90 for 48 h. Controls were incubated accordingly. RiboMab02.1, produced in Balb/cJRj mice, efficiently mediated target-specific and dose-dependent cellular cytotoxicity with EC₅₀ values in the range of 0.004 to 0.200 ng/mL (0.04 to 2.00 pM) for PA-1 and of 0.07 to 2.07 ng/mL (0.7 to 20.7 pM) for OV-90 cells (FIGS. 3 A and B, respectively), depending on the activity of the donor PBMCs.

RiboMab02.1-containing mouse serum did not induce target-independent (non-specific) lysis with the CLDN6-negative MDA-MB-231 cell line (FIG. 3).

### In Vitro-Expressed RiboMab02.1 Leads to Dose-Dependent and Target-Specific T-Cell Proliferation

HEK-293T-17 producer cells were transfected with BNT142 or a control RiboMab-encoding RNA. Supernatants containing RiboMab02.1 or the control RiboMab (specific for an irrelevant TAA) were used as test items. Two concentrations (100 or 1 ng/mL) of RiboMab-containing supernatant were added to co-cultures of carboxyfluorescein succinimidyl ester (CFSE)-labeled human PBMCs and target-positive or -negative cells. PA-1 or OV-90 served as CLDN6-positive and MDA-MB-231 as target-negative cells. CLDN6-negative NUGC-4 cells (expressing the irrelevant TAA) as well as PBMCs in the absence of any target cells were included as further specificity controls. PBMCs from three healthy donors were compared in this assay. After 72 h of incubation, T cells were analyzed by flow cytometry to determine target-dependent proliferation. In the presence of PA-1 cells, approximately 58±2% and 44±7% T-cell proliferation was induced by 100 ng/mL and 1 ng/mL RiboMab02.1, respectively, while with OV-90 cells, 24±3% and 2±3% T-cell proliferation was induced (FIG. 4). In contrast, no RiboMab02.1-driven T-cell proliferation was seen in PBMCs co-cultured with NUGC-4 or MDA-MB-231 cells, nor in the absence of any target cells (FIG. 4). The human CD3-specific antibody OKT3, used as a positive control for T-cell activation, led to strong activation in the presence as well as absence of target cells.

### RiboMab02.1 Elicits Target-Independent T-Cell Activation Only at Doses Far Above the EC₅₀

To determine the range of target-specific bioactivity for RiboMab02.1, T-cell activation as the primary mode-of-action was used as a readout. RiboMab02.1-containing as well as mock serum samples were obtained from mice administered either with BNT142 or luciferase encoding RNA-LNP (Luc_RNA-LNP; mock). Human PBMCs from three healthy donors were used as effector cells. Serum samples were serially-diluted (10-fold, 10-point), starting at 4,000 ng/mL, and added to PBMCs cultured alone or together with CLDN6-positive PA-1 target cells at an effector-to-target-cell ratio of 10:1. After 48 h of incubation, T cells were analyzed by flow cytometry for surface expression of the early and late activation markers CD69 and CD25, respectively. No off-target T-cell activation was seen among PBMCs from any of the donors up to 40 ng/mL RiboMab02.1. The two highest RiboMab02.1 concentrations of 400 and 4,000 ng/mL led to off-target T-cell activation (above background level) of 2 to 6% and 6 to 19%, respectively (FIG. 5, left). The mean EC₅₀ value of all three donors (FIG. 5, right) was determined as 0.026 ng/mL (0.3 pM) in the presence of target cells. OKT3 positive control led to >50% T-cell activation with and without target cells (data not shown). Upregulation of CD69 on T cells was higher than that of CD25; CD25-positive cells were almost exclusively CD69 double-positive, pointing to an early activation state (data not shown).

In conclusion, RiboMab02.1 mediates a target-dependent activation of T cells up to concentrations >1,500-fold above the EC₅₀ value.

### Intravenously Administered BNT142 Mediates Xenograft Tumor Elimination in Mice by Redirection of T Cells to the Tumor

To determine the anti-tumor efficacy of BNT142 in vivo, a CLDN6-positive human ovarian carcinoma (OV-90) xenograft mouse model was used. Briefly, immunodeficient male and female NOD.Cg-Prkd^{scid} IL2rg^{tm1Wjl}/SzJ (NSG) mice were subcutaneously (SC) inoculated with human OV-90 ovarian carcinoma cells. Mice with established tumors (mean size ≥30 mm³) were engrafted intraperitoneally (IP) with human PBMCs from a healthy donor (resulting mouse model annotated as NSG/PBMC). Seven days later, a treatment regimen comprising five weekly single IV bolus injections of BNT142 or control items was initiated. BNT142 was administered at doses of 0.1 or 1 μg (˜0.004 and 0.04 mg/kg) per mouse. As target specificity control, 1 μg control RNA-LNP encoding an irrelevant TAA×CD3-binding tribody protein was applied. Dulbecco's phosphate-buffered saline (DPBS, saline) alone and 1 μg Luc_RNA-LNP served as negative controls, and a recombinant purified CD3×(CLDN6)₂ tribody reference protein (100 μg per animal, ˜4 mg/kg) as positive control (FIG. 6 A).

Tumor volumes started to shrink with 1 μg BNT142 after the second treatment and with 0.1 μg BNT142 or 100 μg of the reference protein after the third treatment. A steady decline was maintained until the end of the experiment (FIG. 6 B, C). None of the negative controls, in general, exhibited tumor volume reduction.

Four mice per group (five mice only for the 0.1 μg BNT142 group) were euthanized 72 h after the third injection to assess tumor-infiltrating lymphocytes (TIL) in the xenograft tumors. On average, 1,310 and 1,797 TIL per mm² of xenograft tumor tissue were seen in mice treated with 0.1 and 1 μg BNT142, respectively (FIGS. 6 D and F). Reference protein treatment resulted in an average 1,542 TIL per mm² tumor tissue while the negative control groups exhibited between 139 and 410 TIL per mm² tissue. Percentages of CLDN6-positive cells in tumor tissue were analyzed for several mice at different time points after Day 38 post OV-90 inoculation. The lowest percentage of CLDN6-positive cells (indicative of increased tumor cell killing/elimination) was found in tumor xenografts from mice treated with 1 μg BNT142, followed by the reference protein and 0.1 μg BNT142 treatments (FIGS. 6 E and F).

Taken together, mice in the BNT142- and reference protein-treated groups showed significant tumor reduction concomitant with higher T-cell infiltration and lower CLDN6 positivity in xenograft tumors in contrast to the negative control groups.

### Secondary Pharmacodynamics

Secondary PD studies are defined as those in vitro and in vivo studies that investigate the mode-of-action and effects of the encoded bispecific protein RiboMab02.1 that are not related to its therapeutic target CLDN6.

### RiboMab02.1 Induces Target-Dependent Release of Human Pro-Inflammatory Cytokines In Vitro

Cell culture supernatants obtained from the T-cell activation assay were analyzed to investigate the RiboMab02.1-dependent induction of on- and off-target cytokine release. The levels of human pro-inflammatory cytokines IFN-γ, IL-1β, IL-2, IL-6, IL-10 and TNF-α were measured using a customized multiplex ELISA kit. RiboMab02.1 induced the release of all cytokines in the presence of target cells in a dose- and donor-dependent manner. Except for a low induction of IL-6 seen with one donor and at the highest concentration of RiboMab02.1, no cytokine induction was observed in the absence of CLDN6-positive PA-1 target cells (FIG. 7). The positive control OKT3 induced cytokine release in the presence as well as absence of target cells, while the mock serum (from Luc_RNA-LNP-administered mice) did not induce any cytokine production (data not shown).

In Vivo Administration of BNT142 to PBMC-Humanized Mice does not Induce Human Pro-Inflammatory Cytokine Release

The human pro-inflammatory cytokines IFN-γ, IL-1β, IL-2, IL-6, IL-10 and TNF-α were measured in serum samples of OV-90 human xenograft tumor-bearing NSG/PBMC mice obtained after 6 and 72 h of the third BNT142 administration (see above). An additional group of tumor-free NSG/PBMC mice was treated with 1 μg BNT142. No cytokine induction was seen in the BNT142-treated or control groups at either time point evaluated (FIG. 8 A), whereas mice in the reference protein group showed transient but significant elevation of all six cytokines at the 6-hour time point. Data were normalized to the values obtained with the saline control group at the respective time points. Systemic availability of the active drug RiboMab02.1 (encoded by 1 μg BNT142) as well as the reference protein was comparable (FIG. 8 B).

### GLP-Compliant Tissue Cross-Reactivity Study

In order to investigate potential non-specific binding of RiboMab02.1, a full human tissue cross reactivity (TCR) study using the parental chimeric IgG antibody IMAB206-C46S (which shares the identical anti-CLDN6 CDRs) at three concentrations (10, 5, 1 μg/mL) was conducted in compliance with GLP. The analyzed adult normal human tissues (FDA 1997) and the findings are summarized in Table 3Error! Reference source not found.Error! Reference source not found.Error! Reference source not found.

TABLE 3
Results from GLP-compliant TCR study

| | Tissues (no. positive |
|---|---|
| Observation | samples/samples tested) |
| Tissues where three | Adrenal (endocrine cells 1/3), |
| concentrations of parental | placenta (villi 1/3) |
| antibody IMAB206-C46S | |
| (which shares the | |
| identical anti-CLDN6 | |
| CDRs with RiboMab02.1) | |
| identified positive | |
| binding and the control no | |
| binding | |
| Tissues where there was | Blood (3/3), |
| no binding identified | Breast (3/3), |
| in any concentration | Cervix (3/3), |
| of parental antibody | Eye (3/3), |
| | Fallopian tube (3/3), |
| | Heart (3/3), |
| | Kidney (3/3), |
| | Lung (3/3), |
| | Lymph node (3/3), |
| | Ovary (3/3), |
| | Pancreas (3/3), |
| | Parathyroid (3/3), |
| | Prostate (3/3), |
| | Spleen (3/3), |
| | Striated muscle (3/3), |
| | Tonsil (3/3), |
| | Uterus-endometrium (3/3) |
| Tissues where binding | Cerebellum (white matter 3/3), |
| was observed in one or | Cerebral cortex (white matter 3/3), |
| more concentrations of | Liver (hepatocytes 2/3), |
| parental antibody and | Peripheral nerve (fiber 2/3), |
| the control | Posterior pituitary (pars novosa 1/3), |
| | Salivary gland (ducts 1/3), |
| | Spinal cord (neural tissue 3/3), |
| | Testis (seminiferous tubes 2/3), |
| | Thyroid (follicular epithelium 1/3), |
| | Ureter (urothelium 1/3), |
| | Urinary bladder (urothelium 2/3), |
| | Bone marrow (macrophages 2/3), |
| | Colon (macrophages 2/3), |
| | Ileum (macrophages 3/3), |
| | Stomach (macrophages 3/3), |
| | Thymus (macrophages 2/3), |
| | Duodenum (epithelial mucosa 1/3), |
| | Skin (epithelium 1/3) |

In brief, out of all tested tissues only endocrine cells of the adrenal gland and villi in placenta stained positive in ⅓ donors at all three concentrations of the test item applied. No membrane staining could be detected, and all observed binding was of cytoplasmic nature. Cytoplasmic binding is only possible because the cell compartments have been artificially exposed by the sectioning process, allowing access of the test item. This situation would not occur in vivo and therefore the cytoplasmic binding is not considered relevant. No other cross-reactivity or non-specific binding was observed.

### Off-Target Binding Assay (Non-GLP)

Cell microarray screening with the tribody reference protein (sequence-identical to RiboMab02.1) was conducted to investigate the potential non-specific targets of RiboMab02.1. The tribody was screened for binding against fixed human HEK293 cells, individually overexpressing 6,239 different full-length human plasma membrane proteins and cell surface-tethered human secreted proteins, including 371 heterodimers.

The tribody reference protein showed specific interaction with its primary targets CD3E, when expressed as a heterodimer with either CD3δ or CD3γ, and CLDN6. Despite this, additional weak off-target interaction with CLDN9 was observed. No other binding was observed.

### Safety Pharmacology

ICH guideline S7A indicates that a core battery of safety pharmacology studies should be performed on any pharmaceutical product before human exposure. The potential effects of up to ˜5 mg/kg RNA-LNP on the central nervous system (CNS) and respiratory system were evaluated as part of the GLP-compliant repeated-dose toxicity study conducted in mice. In addition, the effect of up to 0.15 mg/kg BNT142 on cardiovascular safety endpoints (blood pressure, heart rate and electrocardiogram parameters) were assessed in a non-GLP study in cynomolgus monkeys. In the mouse studies, a control arm treated with empty LNP was included.

### Respiratory Safety

Respiratory safety of RNA-LNP was assessed on satellite animals included in a GLP-compliant repeated-dose toxicity study conducted in Balb/c mice. Four weekly injections were administered and whole-body plethysmography was performed pre-dose and 4 and 24 h post second and fourth injection.

Mice were allocated to four groups, each containing four males and four females. Groups 1 and 2 were control groups and received saline (Group 1) and empty LNPs without RNA but with an equivalent lipid content as the ˜5 mg/kg group (Group 2). Treatment groups received RNA-LNP at doses of ˜1.5 mg/kg (Group 3) and ˜5 mg/kg (Group 4). Plethysmography revealed no test item-related effects.

### CNS Safety

Neurological, behavioral and autonomic effects induced by RNA-LNP were studied at two dose levels (˜1.5 mg/kg and ˜5 mg/kg) in Balb/c mice in compliance with GLP.

This study was conducted on the same animals (same RNA-LNP dose groups and control groups) used in respiratory safety study. The influence of RNA-LNP on 40 neuropharmacological parameters was examined before and 48 h after first dosing, as well as before and 48 h after the fourth dosing.

A test item-related transient decrease in hind-limb grip strength was noted after the first dosing of ˜5 mg/kg in male animals (˜-37%). This effect was also seen in female animals of the same group (˜-4%). The decrease was reversible as no test item-related differences were observed in hind-limb grip strength before or after the fourth dosing. No change in forelimb grip strength was seen at all tested time points and no macroscopic or microscopic changes in the muscle or the sciatic nerve were detected indicating that the transient decrease in hind-limb grip strength was not caused by muscle or nerve damage. No other test item-related effects were observed.

### Cardiovascular Safety

The investigation of potential cardiovascular effects of BNT142 administration has been included in the non-GLP PK and tolerability study in cynomolgus monkey.

In this study, cynomolgus monkeys were allocated to four groups each containing three female animals. Group 1 received saline whereas BNT142 at doses of 0.015, 0.05 and 0.15 mg/kg was administered once to Groups 2, 3 and 4, respectively. Blood pressure measurements were performed before the start of treatment and 6 h and 24 h after dosing of the animals. The peripheral arterial systolic and diastolic blood pressure as well as the resulting mean blood pressure were within the normal physiological limits in the test item-treated animals. In addition, an electrocardiogram (ECG) was recorded starting pre-dose to at least 20 h following dosing. In this interval, the heart rate and ECG quantitative and qualitative parameters were unaffected by the administration of BNT142.

### Non-Clinical Pharmacology—Conclusions

In primary and secondary in vitro as well as in vivo PD studies, BNT142-encoded RiboMab02.1 demonstrated a dose- and target-dependent mode-of-action. The onset of unspecific T-cell activation in vitro by RiboMab02.1 concentrations >1,500-fold above the EC₅₀ (26 μg/mL) points to a broad therapeutic window.

The safety of RiboMab02.1 was tested first in a GLP-compliant tissue cross reactivity study (using a surrogate IgG with identical anti-CLDN6 CDRs) which resulted in no identification of non-specific binding. In a subsequent cell microarray study using a RiboMab02.1 reference protein, weak off-target binding was observed only to CLDN9, which is closely related to CLDN6. Due to the absence of CLDN9 expression from the vast majority of normal tissues, except for expression at tight junctions in the inner ear, unwanted off-target effects are not expected.

Taken together, binding of RiboMab02.1 to targets other than CLDN6 and CD3 is considered unlikely.

Safety pharmacology evaluation revealed no effects on cardiovascular parameters (cynomolgus monkeys) or respiratory parameters (mice). A temporary decrease in hind-limb grip strength was observed 48 h after the first administration of ˜5 mg/kg RNA-LNP in mice. This effect was transient, and was not detected after the three subsequent RNA-LNP administrations. No neurological or muscle abnormalities were found during pathological examination of tissues suggesting that these effects were distinct from neurotoxicity.

Taken together, these data suggest that BNT142-encoded RiboMab02.1 is target-restricted and biologically active in non-clinical models both in vivo and in vitro. No test item-mediated effects on physiological functions of mice have been observed at doses up to ˜5 mg/kg RNA-LNP, covering the anticipated clinical dose range of doses up to 0.15 mg/kg and beyond.

### 2.2 Non-Clinical Pharmacokinetic Studies

The PK of BNT142 can be divided into two parts. First, after IV injection, the RNA-LNPs are distributed systemically and deliver the RNA cargo to the intended target organ, the liver. Second, liver cells transfected by the LNP translate the RNA and secrete the encoded protein RiboMab02.1 into the systemic circulation.

To assess the first part, the biodistribution of radiolabeled LNP in different organs in mice was analyzed. For the evaluation of the second part, modified RNA encoding firefly luciferase formulated in LNPs was utilized to assess liver targeting and kinetics of in vivo translated RNA. Furthermore, the in vivo PK profile of the secreted RiboMab02.1 has been characterized in mice and cynomolgus monkeys (NHP model) after single IV administration.

PK profiling of the PEG lipid excipient (ALC-0159) in the LNP and evaluation of the metabolism of ALC-0159 was evaluated in vivo and in vitro. In vivo, ALC-0159 distributes rapidly from the plasma to the liver, whereas the metabolism of ALC-0159 appears to occur slowly in vitro and in vivo. ALC-0159 is metabolized by hydrolytic metabolism of the ester and amide functional groups, respectively, and this hydrolytic metabolism is observed across all the test species evaluated (mouse, rat, monkey, human). While there was no detectable excretion of lipid in the urine, the percentage of dose excreted unchanged in feces was ˜50% for ALC-0159. Stability of the aminolipid ALC-0366 was evaluated using in vitro metabolism studies. ALC-0366 was stable over 120 min in liver microsomes, S9 fractions and 240 min in hepatocytes in all species.

### Distribution

Biodistribution of LNP in vivo was studied using an RNA-LNP, which has the same LNP formulation as BNT142 but with different RNA. Radiolabeled RNA-LNP was administered IV to mice. LNP distribution in murine tissues was quantified via liquid scintillation spectrometry. Organ targeting of LNPs and subsequent expression of luciferase encoding mRNA was studied via bioluminescence imaging.

### Distribution of Radiolabeled LNPs

The tissue distribution profile of the LNP was investigated in CD-1 mice (n=4/sex/time point) after a single IV bolus injection of 1 mg/kg (based on RNA content). For this study, an RNA-containing LNP was labeled with a non-exchangeable, non-metabolizable lipid marker, [³H]-cholesteryl hexadecyl ether ([³H]-CHE), to monitor the distribution of the lipid particles. Mice were euthanized and blood, plasma and tissues were collected at 0.083 (5 min), 0.25, 0.5, 1, 2, 4, 8 and 24 h post-dose. Radioactivity in all samples was determined by standard liquid scintillation counting and the resulting values used to calculate total lipid equivalent (lipid_{eq}) concentrations and percentage of injected dose in the various tissues.

The LNP exhibited bi-phasic kinetics in blood and plasma in mice, with a rapid initial decline in blood/plasma concentrations, followed by a slow elimination phase. Similar blood/plasma concentration-time profiles were observed in male and female mice. The distribution of LNP into tissues was generally rapid, with peak levels occurring or plateaus achieved in most tissues earlier than 4 h post injection. The liver was the principal organ of distribution, with mean concentrations of 282 and 355 μg lipid_{eq}/g tissue for male and female mice, respectively, at 4 h post injection, which was approximately 70% to 74% of the injected dose. The LNP also distributed into the spleen by 4 h post-dose, resulting in levels of 61.7 to 77.1 μg lipid_{eq}/g tissue (0.84% to 1.15% of the injected dose) at 4 h post-dose. Minimal or no distribution was observed in other tissues. This study was performed to identify the primary organs of LNPs distribution and did not account for total mass balance of the LNP administered.

In summary, the LNP is rapidly and primarily distributed to the liver.

### Liver Targeting and Kinetics of In Vivo Translated RNA

Biodistribution of protein expression was assessed with modified mRNA encoding firefly luciferase formulated with LNPs to assess liver targeting and kinetics of in vivo translated mRNA. Following IV administration, the luciferase protein showed a time-dependent translation with high bioluminescence signals mainly located in the liver (FIG. 9). As the secondary distribution organ, the spleen displayed bioluminescence signals with a factor of ten lower compared to the liver. Minor luminescence signals were detected in heart, lungs, kidneys and lymph nodes.

### Pharmacokinetics of Lipid Excipient (ALC-0159)

BNT142 contains the PEG lipid excipient ALC-0159 in the nanoparticle. Following a single IV dose of a luciferase encoding RNA formulated in an LNP with similar lipid composition (different amino lipid) at 1.96 mg ALC-0159/kg to Wistar Han rats, plasma concentrations of ALC-0159 decreased rapidly, with initial ty, values of 1.72 h. ALC-0159 was then cleared from plasma with a terminal elimination ty, of 72.7 h. The estimated percent of lipid dose distributed to the liver was ˜20%.

### RiboMab02.1 In Vivo Pharmacokinetics

RiboMab02.1 is Efficiently Translated, Assembled and Secreted after Administration of BNT142 in Mice

The following study confirms the translation, assembly and secretion of RiboMab02.1 after uptake of BNT142 in vivo (Balb/cJRj mice). Serum, containing secreted RiboMab02.1, was harvested 2 and 6 h after a single IV administration of 30 μg BNT142. Serum was analyzed by ELISA (FIG. 10 A) and western blot (FIG. 10 B), confirming the generation of fully-assembled and structurally stable (monomeric) RiboMab02.1 in vivo.

### Repeated-Dose PK Study of BNT142 in Mice

A repeated-dose PK study was performed in Balb/cJRj mice to assess whether systemic levels of RiboMab02.1 are sustainable by weekly administration of BNT142. Treatment groups received five IV bolus injections of 10 or 30 μg BNT142 (approximately 0.4 and 1.2 mg/kg, respectively) or 30 μg Luc_RNA-LNP at a weekly interval. Serum samples were prepared from blood drawn from the animals at 6 h after (tₘₐₓ) and 24 h prior to (last detectable concentration [C_{trough}]) each BNT142 administration. Time points correspond to 6, 174, 342, 510, and 678 h (Cₘₐₓ) or 144, 312, 480 and 648 h (C_{trough}) after the first BNT142 or Luc-RNA-LNP control injection, respectively. The last blood samples were drawn 816 h (34 days) after the first injection. RiboMab02.1 peak levels (Cₘₐₓ) of up to 7.4 μg/mL and 46 μg/mL were reached 6 h after the last injection with 10 μg and 30 μg BNT142, respectively, as determined by ELISA. Antibody concentrations rapidly declined within 7 days of each injection, with C_{trough} values approximately 130-fold lower than the Cₘₐₓ values. Importantly, no loss in RiboMab02.1 translation was observed between subsequent injections of either BNT142 dose (FIG. 11).

### Single-Dose PK Study of BNT142 in Cynomolgus Monkeys

A single-dose PK study was performed in female cynomolgus monkeys with three animals per group. Treatment groups received a single IV bolus dose of BNT142 covering the anticipated clinical dose range (0.150, 0.05, and 0.015 mg/kg) or saline as control. Concentrations of RiboMab02.1 were analyzed by ELISA in serum samples collected at different time points (0, 1, 3, 6, 12, 24, 48, 72, 168, and 336 h post injection) to assess dose exposure. RiboMab02.1 peak concentrations (Cₘₐₓ) were reached 6 h (median tₘₐₓ) post BNT142 administration in all the groups (mean values [median tₘₐₓ]: 0.015 mg/kg group=7.3 ng/mL; 0.050 mg/kg group=27.5 ng/mL; 0.150 mg/kg group=155.0 ng/mL). RiboMab02.1 levels were detectable from 3 h and up to 72 h after BNT142 administration in the 0.015 mg/kg group, and up to 168 h (7 days) in the 0.15 mg/kg as well as 0.05 mg/kg groups (FIG. 12 and Table 4). The effective half-life of RiboMab02.1 was determined to be between 27 and 37 hours. However, RiboMab02.1 half-life determination in the two lower dose groups was limited due to the lower limit of quantification (LLOQ) of 0.1 ng/mL of the ELISA quantification method. No signals were detected in the saline control samples or at 336 h (14 days) post-BNT142 administration (detection potentially limited by LLOQ). PK parameters for individual animals and mean/median values are summarized in Table 4. In summary, all three BNT142 doses yielded titers within the therapeutic range of biologically active RiboMab02.1 in cynomolgus monkeys, with a median Cₘₐₓ at 6 h post injection. Inter-individual variability in RiboMab02.1 concentration was observed, with a difference of 2- to 7-fold in the lowest, 1- to 2-fold in the middle and 2-fold in the highest dose groups, respectively, at Cₘₐₓ.

TABLE 4
PK parameters of RiboMab02.1 in cynomolgus monkey serum
following a single intravenous bolus administration of BNT142

| BNT142 | | | | | | Terminal | Effective |
|---|---|---|---|---|---|---|---|
| dose | Subject | tₘₐₓ | Cₘₐₓ | C_{trough} | tₗₐₛₜ | t_{1/2} | t_{1/2} ^{a} |
| (mg/kg) | ID | (h) | (ng/ml) | (ng/ml) | (h) | (h) | (h) |
| 0.015 | 2501 | 6 | 7.3 | 1.5 | 72 | NR | NR |
| | 2502 | 12 | 2.2 | 1.1 | 24 | NC | NC |
| | 2503 | 6 | 12.4 | 2.2 | 72 | 25.1 | 26.5 |
| | n | 3 | 3 | 3 | 3 | 1 | 1 |
| | Mean ^{b} | 6 | 7.3 | 1.6 | 72 | 25.1 | 26.5 |
| | SD | N/A | 5.1 | 0.6 | N/A | N/A | N/A |
| 0.050 | 3501 | 6 | 32.8 | 0.6 | 168 | 27.7 | 28.1 |
| | 3502 | 6 | 19.1 | 4.8 | 72 | NR | NR |
| | 3503 | 12 | 30.7 | 5.6 | 72 | 26.0 | 28.1 |
| | n | 3 | 3 | 3 | 3 | 2 | 2 |
| | Mean ^{b} | 6 | 27.5 | 3.6 | 72 | 26.8 | 28.1 |
| | SD | N/A | 7.4 | 2.7 | N/A | N/A | N/A |
| 0.150 | 4501 | 12 | 242.0 | 15.1 | 168 | 42.1 | 40.1 |
| | 4502 | 6 | 107.0 | 4.8 | 168 | 39.6 | 37.9 |
| | 4503 | 6 | 117.0 | 3.0 | 168 | 32.7 | 31.8 |
| | n | 3 | 3 | 3 | 3 | 3 | 3 |
| | Mean ^{b} | 6 | 155.0 | 7.6 | 168 | 38.1 | 36.6 |
| | SD | N/A | 75.4 | 6.6 | N/A | 4.8 | 4.3 |

^{a.} MRT_{0-inf} (drug mean residence time zero to infinity) was used to calculate effective t_{1/2}.
^{b.} Median values for tₘₐₓ and tₗₐₛₜ.
Cₘₐₓ = maximum observed concentration;
C_{trough} = last detectable concentration;
h = hours;
n = number;
N/A = not applicable;
NC = not calculable;
NR = not reported (the coefficient of determination was less than 0.8);
SD = standard deviation;
t = time;
t_{1/2} = half-life;
tₗₐₛₜ = time of last measurable concentration;
tₘₐₓ = time to maximum concentration observed.

### Metabolism and Excretion

RNA, including N1-methylpseudouridine modified mRNA, is sensitive to degradation by cellular RNases and subjected to nucleic acid metabolism. Nucleotide metabolism occurs continuously within the cell, with the nucleoside being degraded and excreted or recycled for nucleotide synthesis.

Of the four lipids used as excipients in the LNP formulation, two are naturally occurring (cholesterol and DSPC) and will be metabolized and excreted like their endogenous counterparts. The metabolism and excretion of the non-natural lipids (ALC-0366 and ALC-0159) were studied in vitro and in case of ALC-0159 also in vivo.

The in vitro metabolic stability of ALC-0366 (aminolipid) was evaluated in mouse, rat, monkey, and human liver microsomes, S9 fractions, and hepatocytes. ALC-0366 was stable over 120 min in liver microsomes (remaining ALC-0366 >89%) and S9 fractions (remaining ALC-0366 >88%) and over 240 min in hepatocytes (remaining ALC-0366 >94%) in all species and test systems.

The metabolism of ALC-0159, was examined in vitro using blood, liver S9 fractions and hepatocytes, all from mouse, rat, monkey and human and ex vivo in the rat plasma, urine, feces, and liver from the PK study. These studies determined that ALC-0159 is metabolized relatively slowly by amide bond hydrolysis yielding N,N-ditetradecylamine. This hydrolytic metabolism was observed across the species evaluated.

In the rat PK study, there was no detectable excretion of ALC-0159 in urine after IV administration of RNA-LNP. However, ˜50% of ALC-0159 was eliminated unchanged in feces.

### Non-Clinical Pharmacokinetics and Metabolism—Summary and Conclusions

The PK of BNT142 can be divided into two parts:
PK of BNT142: After IV injection, the RNA-LNPs are distributed systemically within 4 h and deliver the RNA cargo to the intended target organ, the liver.
PK of the BNT142-encoded bispecific antibody RiboMab02.1: After delivery into liver cells, the RNA is translated and the encoded RiboMab02.1 protein is released into the circulation.

To investigate the first parts, a biodistribution study was performed in mice with an LNP containing a radiolabeled lipid. The distribution of the LNP was generally rapid, with peak levels achieved in most tissues earlier than 4 h post injection. The liver was the principal organ of distribution followed by the spleen, where just a fraction of the LNP was distributed to, and minimal or no distribution to other tissues was observed. Targeted RNA translation in the liver was shown by in vivo imaging following administration of LNP-formulated luciferase (Luc)-encoding RNA in mice. Luc translation (bioluminescence) was observed in the liver for up to 144 h post injection, while the spleen displayed ten-fold lower signal intensity.

In a rat IV PK study, concentrations of PEG lipid (ALC-0159) dropped approximately 8,000 fold in plasma during this 2 wk study. The apparent terminal ty, in plasma was 72.7 h for ALC-0159 and it likely represents the re-distribution of the lipid from the tissues which initially took up the LNPs back to plasma for elimination, mostly unchanged in feces. In vitro, ALC-0159 was metabolized slowly by hydrolytic metabolism of the amide functionality, while in vitro metabolism studies of the non-natural amino lipid (ALC-0366) indicated stability in all species and test systems.

PK parameters of the BNT142-encoded bispecific antibody RiboMab02.1 were determined via a repeated-dose study in mice and single-dose study in NHPs through quantitation of translated RiboMab02.1 in serum.

Dose-dependent RiboMab02.1 serum concentrations were detectable in mice dosed weekly with BNT142. A Cₘₐₓ of 46 μg/mL was reached after the 5^{th} dosing in the 30 μg (˜1.2 mg/kg) dose cohort, affirming RiboMab02.1 exposure recovery.

In a single dosing PK study in NHPs, RiboMab02.1 displayed a dose-dependent expression with a median tₘₐₓ at 6 h post-administration (as observed in mice) and a mean Cₘₐₓ of 155 ng/mL after dosing with 0.15 mg/kg BNT142 (highest dose tested). RiboMab02.1 was detectable in NHPs until the end of the study 7 days post dosing.

In summary, PK data in mice and NHPs demonstrated a BNT142 dose-dependent, but not dose-proportional, exposure of RiboMab02.1. Peak RiboMab02.1 concentrations were reached 6 h post dosing, declining thereafter. As the non-clinical effective half-life of RiboMab02.1 is 27 to 37 h, full clearance of the antibody from the human body is anticipated to occur in approximately five half-lives (˜135 to 185 h or 6 to 8 days). Therefore, weekly dosing will support maintenance of a RiboMab02.1 plasma level that confers biological activity throughout the q1w clinical treatment interval.

### 2.3 Toxicology

The non-clinical (immune) toxicology program assessing RNA-LNP-mediated effects used a platform approach, including in vitro studies using human cells and blood components, and in vivo studies in mice and cynomolgus monkeys.

In a non-GLP single-dose toxicity study in mice, a dose of up to 4 mg/kg RNA-LNP was tolerated. Findings included clinical observations (piloerection and dehydration), minimal bodyweight loss and delayed body weight gain, small increases in liver transaminases, increased spleen weight and minimal to mild mixed leukocyte infiltration in liver, consistent with minimal hepatic injury, and a mild inflammatory response. All effects were reversible within 28 days.

In the repeated-dose toxicity study, RNA-LNP doses of up to ˜5 mg/kg were tolerated in mice. Findings in the study included a small decrease in food consumption, a transient decrease in total white blood cells and transient alkaline phosphatase and liver enzyme elevations. Furthermore, an increase of kidney and liver weight were noted in addition to reversible microscopic changes in the liver at doses of 5 mg/kg, as well as extramedullary hematopoiesis in the spleen and lymphoid hyperplasia of the periarteriolar lymphoid sheath, which was also associated with an increase in the spleen weight. These changes ameliorated and in most cases reversed fully after a 2 wk recovery period.

In the non-GLP PK/tolerability study in NHPs testing a single dose of up to 0.15 mg/kg BNT142, no changes were observed in any parameter.

In immunotoxicity assessments, ˜1.5 mg/kg and ˜5 mg/kg RNA-LNPs induced a transient elevation of cytokines (IFN-α, IFN-γ, IL-6, and TNF-α) 6 h post-administration in mice, which was less pronounced after repeated administration. No BNT142-induced cytokine elevation was observed in vivo in cynomolgus monkeys after a single administration of up to 0.15 mg/kg. In in vitro studies using human whole blood, BNT142 at concentrations ≥4 μg/mL (human equivalent dose of 0.32 mg/kg) induced secretion of IFN-γ, IL-1β-, IL-2, IL-6, IL-8 and TNF-α. No RNA-LNP activation of complement system was observed in vitro for concentrations of up to 40 μg/mL (human equivalent dose of 3.2 mg/kg).

### Selection of Relevant Species for Safety Studies for RiboMab02.1

According to ICH S6 (R1) a relevant species is one in which the test material is pharmacologically active. Pharmacological activity of RiboMab02.1 bispecific antibody requires binding of the CD3 receptor on T cells of the respective species and the expression of the tumor target epitope (CLDN6). However, the anti-CD3 part of RiboMab02.1 is not cross-reactive to CD3 of any other species but human primates.

As there is no suitable test species to adequately capture the safety profile of RiboMab02.1, no safety studies in animals were conducted for the antibody. Instead, the in vitro safety studies described above were conducted using either the CLDN6 parental antibody IMAB206-C46S or a recombinant reference protein identical to RiboMab02.1.

### Non-Clinical Safety Assessments of the RNA-LNP Drug Product

In some of the RNA-LNP safety studies, a drug product with the identical LNP lipid composition as BNT142 but with a different RNA was used. As differences in the RNA sequence or length are not anticipated to influence the RNA-LNP-mediated safety profile to a large extent, the results are considered to be representative for RNA-LNP-mediated toxicities of BNT142. This platform toxicity assessment approach has been accepted previously.

### Single-Dose Toxicology

A non-GLP single-dose toxicity study was conducted in male and female CD-1 mice. This study characterized the potential toxicity of RNA-LNPs doses of 1 to 4 mg/kg, identified LNP mediated toxicities by comparing the toxicity of RNA-LNP with the respective control item (i.e., empty LNP), and assessed the reversibility, progression and/or potential delayed effects of RNA-LNP after a 4 wk observation period (termination on Day 29).

Mice received a single IV dose of RNA-LNP or control item (saline or empty LNP) on Day 1. Animals were euthanized on Day 3 (main animals) or Day 29 (recovery animals). Study endpoints included mortality, clinical observations, body weight changes, clinical pathology, necropsy observations, organ weights and histopathology (liver, spleen and stomach).

A single dose of up to 4 mg/kg RNA-LNP was tolerated in male and female CD-1 mice. Findings included clinical observations (piloerection and dehydration), minimal bodyweight loss and delayed body weight gain. On Day 3 small increases in liver transaminases (alanine transaminase [ALT] and/or aspartate transaminase [AST]), an increased spleen weight, a pale liver in a single female receiving 4 mg/kg, and minimal mixed leukocyte infiltration in the liver, consistent with minimal hepatic injury and a mild inflammatory response were detectable. There were no differences in findings between sexes. All effects showed evidence of full or partial recovery on Day 29, demonstrated by the mixed leukocyte infiltration in the liver, indicating ongoing resolution of a mild inflammatory response.

### Repeated-Dose Toxicology

A GLP-compliant platform repeated-dose toxicity study assessing LNP- and RNA-mediated toxicities was conducted in male and female Balb/cJRj mice. This study characterized the potential toxicity of four weekly RNA-LNP doses of ˜1.5 and ˜5 mg/kg, identified LNP mediated toxicities by comparing the toxicity of RNA-LNP with the respective control item (i.e., empty LNP), and assessed the reversibility, progression and/or potential delayed effects of RNA-LNP after a 2 wk observation period. The RNA-LNP doses tested (up to ˜5 mg/kg) exceed the anticipated clinical doses (0.00005 mg/kg to 0.15 mg/kg) by a factor of 30 to 30,000×. In the GLP-compliant mouse toxicity study, the highest dose (100 μg/animal equivalent to ˜5 mg/kg) was considered as the maximum feasible dose based on the maximum feasible volume and concentration that could be delivered IV.

Treatment with four weekly IV injections of RNA-LNP was tolerated at doses of 30 and 100 μg/animal (˜1.5 and ˜5 mg/kg, respectively) in mice. In the high dose group, animals showed transiently reduced food consumption (−8.2% in males or −13.6% in females). However, these alterations in food consumption did not result in a concomitant decrease in body weight. No mortality was observed during the treatment or recovery period.

Hematological parameters were assessed 24 h after the third dosing (test Day 16) for main study animals and one wk after the last treatment (test Day 30) for recovery animals. Treatment with 30 μg (˜1.5 mg/kg) or 100 μg (˜5 mg/kg) RNA-LNP led to a decrease in total white blood cells on test Day 16 (−59% or −62%, respectively, in males and −67% or −74%, respectively, in females). This was mainly due to a significant decrease in lymphocytes (−67% or −72%, respectively, in males and −74% or −79%, respectively, in females).

A decrease in absolute number of white blood cells (−24.1% in males and −37.4% in females and lymphocytes (−37.2% in males and −48.7% in females) was also noted for animals treated with empty LNPs. No test item-related differences in hematological parameters were found for recovery animals indicating all changes were reversible.

Clinical chemistry was assessed 24 h after the last dose (test Day 23) and at the end of the recovery period (test Day 37). Fully reversible elevation in liver enzymes ALT (+51%), AST (+45%) and lactate dehydrogenase (LDH) (+69%) were observed in female mice treated with 30 μg (˜1.5 mg/kg) on test Day 23. No other test item-related difference for the biochemical parameters were noted for this dose group. In the high dose (100 μg, ˜5 mg/kg RNA-LNP), an elevation in liver enzymes (ALT, AST) and LDH was observed in the females treated (+8,170%, +4,992% and +2,787% respectively). An increase in liver enzyme activity (ALT, AST) and LDH was also noted in females treated with empty LNPs (+274%, +44% and +40%). In addition, for alkaline phosphatase (ALP) an increase in both sexes of this group (males +39%, females +93%) was observed. The level decreased throughout the recovery period but was still elevated above laboratory normal at the end of the study (males, +19%, females +21%).

Additionally, globulin concentration was increased for males (+31%) and for females (+36%) on test Day 23. Accordingly, a decreased albumin/globulin ratio was noted for the males (−15%) and the females (−21%). These changes had subsided at the end of the recovery period.

On test Day 23, approximately 24 h after the fourth administration, the main study animals were dissected. Necropsy of all recovery animals was performed on test Day 37. At the end of the main study, the spleen was enlarged in some female animals from both dose groups. This effect was associated with an increase in spleen weight (+68% in males and +99% in females). Additionally, necropsy revealed a pale liver for one female and a partly pale liver with a smooth surface for another female, both dosed with 100 μg. In this high dose group in both genders an increased liver (+17% in males and +34% in females) and kidney weight (+15% in males and +18% in females) was also noted. The effect on spleen and liver weight was not fully compensated during recovery but all other effects had subsided.

Histopathological changes were found only in females in the liver (cytoplasmic alteration, mononuclear inflammatory cell infiltrate, multinucleated giant cells and necrosis) at the end of the main study. These findings were fully reversible in all but one female after the recovery period. Fully reversible histopathology findings in the spleen of females were observed in both low and high dose groups, which included extramedullary hematopoiesis, congestions, pigment disposition and lymphoid hyperplasia of the periarteriolar lymphoid sheath. No histopathological observations were made in male animals at any time point.

### Single-Dose Pharmacokinetic and Tolerability Study of BNT142 in Cynomolgus Monkeys

The aim of the study was to obtain information of the PK and tolerability of BNT142 after single-dose IV administration to cynomolgus monkeys.

A single administration of BNT142 by IV bolus injection was tolerated in monkeys at doses of up to 0.15 mg/kg. There were no premature decedents, clinical observations or changes in body weight, body temperature, blood pressure, ophthalmology, auditory examinations, cardiovascular assessments, clinical pathology parameters (hematology, coagulation, clinical chemistry, and urinalysis) or cytokines associated with the administration of BNT142.

### Immunotoxicity Studies

Immunotoxicological parameters were assessed in two stand-alone studies (described below) and as part of the GLP-compliant repeated-dose toxicity study in mice and a single dose PK study in NHPs. In mice, cytokine analyses were performed at 6, 24, and 48 h after first dosing (test days 1, 2, and 3) and also at 6, 24, and 48 h after the third dosing (test days 15, 16 and 17). IFN-α, IFN-γ, IL-6, and TNF-α were transiently elevated at 6 h post-administration in male and female animals in both dose groups (˜1.5 mg/kg and ˜5 mg/kg) on test Day 1 and test Day 15. The extent of elevation was less after repeated administration (test Day 15). The levels of IL-6, and TNF-α had fully declined by 24 h post dosing while levels of IFN-α and IFN-γ returned to baseline by 48 h post-administration. No elevation of IL-1p, IL-2, IL-10, or IL-12p70 was observed in any of the groups. In NHPs, no test item-related cytokine elevations were observed 6, 24 or 48 h post doses of up to 0.15 mg/kg in any of the cytokines tested. No clinical signs indicating systemic immunotoxicity were observed.

### In Vitro Complement Activation of Human Serum

The potential of the drug product to activate human complement in vitro was evaluated through incubation of normal human serum with RNA-LNP concentrations 0.04 to 40 μg/mL (representative of doses of ˜0.003 to ˜3.2 mg/kg), covering the anticipated clinical exposure. No complement activation was observed after incubation with RNA-LNP.

### Whole Blood Cytokine Release by BNT142

Secretion of pro-inflammatory cytokines (IFN-α, IFN-γ, IL-1β, IL-2, IL-6, IL-8, IL-12p70, IP-10 and TNF-α) was evaluated after incubation of BNT142 with whole blood. The dilution range tested (0.0625 to 64 μg/ml) is representative of and exceeding the anticipated concentrations of drug product (equivalent to doses of 0.005 to 5.12 mg/kg). No test item-related cytokine secretion was detectable up to a concentration of 2 μg/mL (human equivalent dose of 0.16 mg/kg). Beyond that, BNT142 induced IL-1β, IL-6, IL-8 at concentrations of ≥4 μg/mL (human equivalent dose of 0.32 mg/kg) and IFN-γ, IL-2, TNF-α secretion at concentrations of ≥32 μg/mL (human equivalent dose of 2.56 mg/kg).

### Toxicology—Conclusions

The toxicology assessment of BNT142 was split into two parts—one part assessing the safety of the encoded antibody RiboMab02.1 in vitro, attributed to the lack of a pharmacologically relevant species where this compound would be active. The RNA-LNP instead was tested in several in vivo and in vitro studies, some of which used a surrogate with the identical type of modified mRNA but with a different sequence (e.g., encoding for luciferase). This approach has proven reliable on other occasions.

The safety of the RNA-LNP has been assessed in three in vivo toxicity/tolerability studies in mice and monkeys. Whereas in the anticipated upper clinical dose range of BNT142 (0.015 to 0.15 mg/kg) no RNA-LNP related effects were observed in an NHP tolerability study, higher single or multiple doses of RNA-LNP (1 to ˜5 mg/kg) in mice led to effects on clinical pathology parameters, organ weights and histopathological changes, mainly in a dose-dependent manner. These effects were often only present or more pronounced in female animals.

In both sexes, lymphocyte counts (and therefore leukocyte counts) were decreased and globulin concentrations increased after repeated administration of ˜1.5 or ˜5 mg/kg RNA-LNP. A single dose of 2 mg/kg (females) and 4 mg/kg (both sexes) induced minimal ALT and AST elevations, which were more pronounced and observed together with LDH elevations after repeated administration in females receiving multiple doses of either ˜1.5 mg/kg or ˜5 mg/kg RNA-LNP. Accordingly, these effects were associated with minimal-mild (single-dose) or minimal-marked (repeated-dose) changes in the liver histopathology (cytoplasmic alteration, mononuclear inflammatory cell infiltrate, multinucleated giant cell(s) and necrosis). Microscopic alterations, such as congestions, extramedullary hematopoesis, lymphoid hyperplasia of the periarteriolar sheath or pigment disposition were also seen in the spleen of females dosed repeatedly with ˜1.5 mg/kg or ˜5 mg/kg RNA-LNP. Although weight increase in the liver, spleen and kidneys were observed in male and female animals, no other histopathological observation was made. The majority of the described effects were fully reversible within 2 to 3 wks, apart from ALP levels which were still slightly elevated in both dose groups and some pathological changes in females treated with higher doses (˜4 mg/kg as a single dose or ˜5 mg/kg as four consecutive doses), which showed amelioration by the end of recovery.

Immunotoxicological assessments of RNA-LNP in vitro indicated no activation of human complement up to a human equivalent dose of ˜3.2 mg/kg but drug product concentration dependent cytokine release at human equivalent doses of 0.3 mg/kg or higher. Supporting these findings, reversible cytokine elevations were seen after administration of ˜1.5 or ˜5 mg/kg RNA-LNP to mice in the GLP toxicity study, though the extent of cytokine elevation was lower after repeated administration. In the NHP tolerability study, no cytokine elevation was seen after a single administration of the upper clinical dose range, 0.015 to 0.15 mg/kg BNT142.

In summary, based on the above discussed non-clinical toxicology results, a first-in-human-dose of 0.00005 mg/kg (0.05 ag/kg) is considered reasonably safe while expected to yield pharmacologically relevant exposure of RiboMab02.1 in humans.

### Example 3: In Vivo Expressed RiboMab02.1 is Highly Monomeric and Induces Lower ADA Response in Mice than the Alternative Lead Structure Candidate RiboMab_712/711 C53W

A free cysteine in the parental anti-CLDN6 VL (VL(CLDN6) at position 53 (C53) according to IMGT numbering) has been substituted to serine (C53S) in RiboMab02.1 (SEQ ID NOs: 4 and 6, encoded by mRNA sequences SEQ ID NOs: 5 and 7, respectively) or to tryptophane (C53W) in RiboMab_712/711 (SEQ ID NOs: 31 and 33, encoded by mRNA sequences SEQ ID NOs: 32 and 34, respectively). Position 53 of the parental anti-CLDN6 VL corresponds to position 449 in sequences SEQ ID NO: 4 and 31 and to position 428 in sequences SEQ ID NO: 6 and 33. It also corresponds to position +15 relative to CDR1 of VL(CLDN6) and/or position −3 relative to CDR2 of VL(CLDN6). Both CD3×(CLDN6)₂ encoding lead structure variants were compared in mice with respect to protein aggregation and induction of RiboMab specific anti-drug antibodies (ADA).

Four female Balb/cJRj mice received an IV single dose of 30 μg RNA-LNP per mouse (˜1.4 mg/kg). Serum was sampled pre- and 6, 24, 48, 96, 168, 216, 265 and 528 hours (0.25 to 22 days) post-administration.

Serum samples obtained 6 hours post-injection were analyzed by western blot analysis under non-reducing conditions (FIG. 13A; representative western blot image of mouse #4 from each group). Equal volumes of Melon G-purified sera were used. RiboMab_712/711 carrying a C₅₃W substitution led to the production of higher amounts of HMW species than the C₅₃S variant (RiboMab02.1) relative to their expression levels. HMW for RiboMab02.1 were hardly detectable in vivo. This is an important difference as drug HMW formation often correlates with increased immunogenicity.

Anti-RiboMab02.1 and anti-RiboMab_712/711 drug antibodies (ADAs) in mouse sera were determined using a Gyros xPand™ XPA1025 ELISA device from Gyros Protein Technologies AB. Sandwich immunoassay was processed on a Gyrolab Bioaffy 200 CD.

Anti-RiboMab ADAs were captured using 100 μg/mL biotinylated drug antibody. Serum ADA levels were detected with 25 nM Alexa Fluor 647-conjugated AffiniPure goat anti-mouse IgG, Fc fragment specific antibody. Data were generated using the 200-3W-002-A method and the results were analyzed using Gyrolab Evaluator software.

The positive ADA response was determined according to the floating cut-point. Therefore, the cut-point was calculated using the response of the negative sera (pre-bleeds) and the formula, normalization factor (NF)+mean negative controls (NC).

NF=X*SD of the negative samples; X=1.645; SD=standard deviation of the negative sera (pre-bleeds); NC=3 negative controls (pooled negative sera).

The data (FIG. 13B) indicate a lower immunogenic potential of RiboMab02.1 in mice compared to RiboMab_712/711, in line with the higher monomeric content of RiboMab02.1 (FIG. 13A). The lower immunogenicity cannot be explained by differences in expression levels as immunogenicity does not tighty correlate with expression levels.

In conclusion, a substitution of the free cysteine C₅₃ by a serine as in both RiboMab02.1 sequences led to a lower level of HMW formation and ADA induction compared to a substitution to a tryptophane as in both sequences of RiboMab_712/711.

### Example 4: An HC:LC Weight Ratio of 1.5:1 of the RiboMab02.1-Encoding Drug Substance Intermediates Results in Efficient Expression of Highly Monomeric RiboMab02.1

HEK-293T-17 producer cells were transfected by electroporation with HC- and LC-encoding drug substance intermediates (RNAs) at HC:LC weight ratios of 1:1, 1.25:1, 1.5:1, 1.75:1, 2:1, 2.25:1, 2.5:1 and 2.75:1 (total RNA concentration: 100 μg/mL RNA; total volume: 250 μL). The HC- and LC-encoding drug substance intermediates (RNAs) used in this example (respectively SEQ ID NO: 5 and 7) encode the two polypeptides of RiboMab02.1 SEQ ID NOs: 4 and 6. The two polypeptides are referred to here as HC and LC because they respectively comprise variable regions derived from a heavy and light chain of a parental antibody that targets CD3. Neither of the polypeptides of RiboMab02.1 comprises a full heavy or light chain of an antibody.

Cell culture supernatant (SN) samples were subjected to Western blot (WB) and ELISA analyses 48 h after transfection (FIG. 14).

For WB analysis (FIG. 14A), SN samples were heat-treated for protein denaturation followed by SDS-PAGE under non-reducing conditions. Two preparations of a reference tribody protein were used as positive control: one containing 0.23% high molecular weight (HMW; monomer reference) species and the other, 96.5% HMW (HMW reference) species. The protein bands separated by SDS-PAGE were then blotted onto a nitrocellulose membrane and incubated with a combination of two HRP-conjugated goat anti-human detection antibodies targeting the kappa light chain or the IgG Fd region, respectively. Following this, the blotted membrane was developed with a chemiluminescent reagent and exposed for 4 seconds. The image was subsequently analyzed by software to quantify the signal intensity and percentage proportion of the individual bands (Table 5). Free LC forms characterized by low molecular weight (LMW) species were consistently low in all SN samples tested. Monomer content was high overall (>90%) and HMW species—primarily HC dimers of 200 kDa—varied between 2.4% to 9.1% across the HC:LC RNA ratios tested.

For RiboMab02.1 quantitation by sandwich immunoassay, SN samples of technical duplicates from two independent experiments were analyzed using a Gyros xPand™ XPA1025 ELISA device using biotinylated or AlexaFlour647-conjugated goat anti-mouse IgG antibodies for capture and detection, respectively. The assay was performed in a Gyrolab Bioaffy 20 HC CD. Data were generated with the Gyrolab huIgG—High Titer method v2 and the results evaluated using Gyrolab Evaluator software.,

The results (FIG. 14B) show that the HC-encoding RNA content was inversely correlated with the amount of monomeric RiboMab detected by ELISA. Accordingly, the HC:LC RNA ratios of 1.25:1 and 1.5:1 yielded the highest RiboMab concentrations in the SN (˜3.5 μg/mL) and, in a dose-dependent manner, the HC:LC ratio of 2.75:1 yielded the lowest amount of RiboMab02.1 (˜2.0 μg/mL). The HC:LC RNA ratio of 1.5:1 was selected for downstream development of RiboMab02.1-encoding drug substance and drug product (Table 5) as it exhibited the most favorable RiboMab02.1 yield-to-HMW content.

TABLE 5
RiboMab2.1 concentration and monomer content after transfection
of various RiboMab02.1-encoding drug substance intermediate ratios

| RiboMab02.1- | | | | |
|---|---|---|---|---|
| encoding | RiboMab02.1 | | | |
| HC:LC | Concentration, | | | |
| RNA weight | mean ± SD | Monomer | HMW | LMW |
| ratio | (μg/μL) | (%) | (%) | (%) |
| 1:1 | 3.21 ± 0.11 | 90.7 | 9.1 | 0.3 |
| 1.25:1 | 3.45 ± 0.34 | 93.4 | 6.3 | 0.3 |
| 1.5:1 | 3.52 ± 0.48 | 94.8 | 4.9 | 0.3 |
| 1.75:1 | 2.86 ± 0.44 | 94.8 | 4.8 | 0.4 |
| 2:1 | 2.53 ± 0.36 | 96.0 | 3.7 | 0.3 |
| 2.25:1 | 2.06 ± 0.11 | 96.6 | 3.1 | 0.3 |
| 2.5:1 | 2.07 ± 0.19 | 96.6 | 3.1 | 0.3 |
| 2.75:1 | 1.74 ± 0.07 | 97.3 | 2.4 | 0.3 |

The HC: LC RNA ratio selected for further development is shown in bold text.
LC = light chain;
LMW = low molecular weight;
HC = heavy chain;
HMW = high molecular weight;
SD = standard deviation

1. A composition or medical preparation comprising:
(i) a first RNA encoding a first polypeptide chain comprising a variable region of a heavy chain (VH) derived from an immunoglobulin with specificity for CD3 (VH(CD3)), a variable region of a heavy chain (VH) derived from an immunoglobulin with specificity for CLDN6 (VH(CLDN6)) and a variable region of a light chain (VL) derived from an immunoglobulin with specificity for CLDN6 (VL(CLDN6)); and
(ii) a second RNA encoding a second polypeptide chain comprising a variable region of a light chain (VL) derived from an immunoglobulin with specificity for CD3 (VL(CD3)), a variable region of a heavy chain (VH) derived from an immunoglobulin with specificity for CLDN6 (VH(CLDN6)) and a variable region of a light chain (VL) derived from an immunoglobulin with specificity for CLDN6 (VL(CLDN6)).

2. The composition or medical preparation of claim 1, wherein
(i) the first polypeptide chain interacts with the second polypeptide chain to form a binding domain with specificity for CD3 and two binding domains with specificity for CLDN6; and/or
(ii) the VH(CD3) of the first polypeptide chain and the VL(CD3) of the second polypeptide chain interact to form a binding domain with specificity for CD3,
the VH(CLDN6) and the VL(CLDN6) of the first polypeptide chain interact to form a binding domain with specificity for CLDN6, and
the VH(CLDN6) and the VL(CLDN6) of the second polypeptide chain interact to form a binding domain with specificity for CLDN6; and/or
(iii) the first and the second polypeptide chains comprise a constant region 1 of a heavy chain (CH1) from an immunoglobulin or a functional variant thereof and a constant region of a light chain (CL) from an immunoglobulin or a functional variant thereof, wherein the CH1 on the first polypeptide chain optionally interacts with the CL on the second polypeptide chain; and/or
(iv) the immunoglobulin is IgG1, optionally human IgG1.

3-6. (canceled)

7. The composition or medical preparation of claim 1, wherein
(i) the VH, the VL, and the CH1 on the first polypeptide chain are arranged, from N-terminus to C-terminus, in the order
VH(CD3)-CH1-VH(CLDN6)-VL(CLDN6), or
VH(CD3)-CH1-VL(CLDN6)-VH(CLDN6); and/or
(ii) the CH1 is connected to the VH(CLDN6) or VL(CLDN6) by a peptide linker, wherein the peptide linker optionally comprises the amino acid sequence SGPGGGRS(G₄S)₂ or a functional variant thereof; and/or
(iii) the VH, the VL, and the CL on the second polypeptide chain are arranged, from N-terminus to C-terminus, in the order
VL(CD3)-CL-VH(CLDN6)-VL(CLDN6), or
VL(CD3)-CL-VL(CLDN6)-VH(CLDN6); and/or
(iv) the CL is connected to the VH(CLDN6) or VL(CLDN6) by a peptide linker, wherein the peptide linker optionally comprises the amino acid sequence DVPGGS or a functional variant thereof; and/or
(v) the VH(CLDN6) and the VL(CLDN6) are connected to one another by a peptide linker, wherein the peptide linker optionally comprises the amino acid sequence (G₄S)ₓ or a functional variant thereof, wherein x is 2, 3, 4, 5 or 6, wherein the peptide linker further optionally comprises the amino acid sequence (G₄S)₄ or a functional variant thereof.

8-16. (canceled)

17. The composition or medical preparation of claim 1, wherein;
(i) the VH(CD3) comprises CDR1, CDR2 and CDR3 of the amino acid sequence of amino acids 27 to 145 of SEQ ID NO: 4; and/or
(ii) the VL(CD3) comprises CDR1, CDR2 and CDR3 of the amino acid sequence of amino acids 27 to 132 of SEQ ID NO: 6; and/or
(iii) the VH(CLDN6) comprises CDR1, CDR2 and CDR3 of the amino acid sequence of amino acids 267 to 383 of SEQ ID NO: 4; and/or
(iv) the VL(CLDN6) comprises CDR1, CDR2 and CDR3 of the amino acid sequence of amino acids 404 to 510 of SEQ ID NO: 4 and preferably a serine residue in position +15 relative to CDR1 and/or a serine residue in position −3 relative to CDR2; and/or
(v) the VH(CD3) comprises CDR1, CDR2 and CDR3 of the amino acid sequence of amino acids 27 to 145 of SEQ ID NO: 4, the VL(CD3) comprises CDR1, CDR2 and CDR3 of the amino acid sequence of amino acids 27 to 132 of SEQ ID NO: 6, the VH(CLDN6) comprises CDR1, CDR2 and CDR3 of the amino acid sequence of amino acids 267 to 383 of SEQ ID NO: 4, and the VL(CLDN6) comprises CDR1, CDR2 and CDR3 of the amino acid sequence of amino acids 404 to 510 of SEQ ID NO: 4 and preferably the VL(CLDN6) comprises a serine residue in position +15 relative to CDR1 and/or a serine residue in position −3 relative to CDR2.

18-21. (canceled)

22. The composition or medical preparation of claim 1, wherein
(i) the VH(CD3) comprises the amino acid sequence of amino acids 27 to 145 of SEQ ID NO: 4 or a functional variant thereof,
the VL(CD3) comprises the amino acid sequence of amino acids 27 to 132 of SEQ ID NO:
6 or a functional variant thereof,
the VH(CLDN6) comprises the amino acid sequence of amino acids 267 to 383 of SEQ ID NO: 4 or a functional variant thereof, and/or
the VL(CLDN6) comprises the amino acid sequence of amino acids 404 to 510 of SEQ ID NO: 4 or a functional variant thereof; and/or
(ii) the first polypeptide chain comprises the amino acid sequence of SEQ ID NO: 4 or a functional variant thereof; and/or
(iii) the second polypeptide chain comprises the amino acid sequence of SEQ ID NO: 6 or a functional variant thereof; and/or
(iv) the first polypeptide chain comprises the amino acid sequence of SEQ ID NO: 4 or a functional variant thereof and the second polypeptide chain comprises the amino acid sequence of SEQ ID NO: 6 or a functional variant thereof; and/or
(v) at least one, optionally each, of the first polypeptide and the second polypeptide is encoded by a coding sequence which is codon-optimized and/or the G/C content of which is increased compared to wild type coding sequence, wherein the codon-optimization and/or the increase in the G/C content preferably does not change the sequence of the encoded amino acid sequence; and/or
(vi) the RNA comprises a modified nucleoside in place of uridine, optionally in place of each uridine, wherein the modified nucleoside is further optionally selected from pseudouridine (ψ), N1-methyl-pseudouridine (m1ψ), and 5-methyl-uridine (m5U).

23-29. (canceled)

30. The composition or medical preparation of claim 1, wherein
(i) at least one, optionally each, RNA comprises the 5′ cap m₂^{7,3′—O}Gppp(m₁^{2′-O})ApG; and/or
ii) at least one, optionally each, RNA comprises a 5′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8; and/or
(iii) at least one, optionally each, RNA comprises a 3′ UTR comprising the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9; and/or
(iv) at least one, optionally each, RNA comprises a poly-A sequence, wherein the poly-A sequence optionally comprises at least 100 nucleotides or comprises or consists of the nucleotide sequence of SEQ ID NO: 10.

31-39. (canceled)

40. The composition or medical preparation of claim 1, wherein
(i) the first RNA and the second RNA are in a (w/w) ratio of about 1.75:1 to about 1.25:1, or about 1.5:1 to about 1.25:1, or preferably about 1.5:1; and/or
(ii) the first RNA and the second RNA comprise a modified nucleoside in place of each uridine; and/or
(iii) the first RNA and the second RNA comprise a modified nucleoside in place of each uridine, wherein the modified nucleoside is independently selected from pseudouridine (ψ), N1-methyl-pseudouridine (m1ψ), and 5-methyl-uridine (m5U); and/or
(iv) the first RNA and the second RNA comprise the 5′ cap m₂^{7,3′O}Gppp(m₁^{2′-O})ApG; and/or
(v) the first RNA and the second RNA comprise a 5′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8; and/or
(vi) the first RNA and the second RNA comprise a 3′ UTR comprising the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9; and/or
(vii) the first RNA and the second RNA comprise a poly-A tail comprising the nucleotide sequence of SEQ ID NO: 10.

41. The composition or medical preparation of claim 1, wherein
(i) the first polypeptide chain comprises the amino acid sequence of SEQ ID NO: 4, or an amino acid sequence having at least 99%, 98%, 97%, 96%, 95%, 90%, 85%, or 80% identity to the amino acid sequence of SEQ ID NO: 4; and/or
(ii) the first RNA comprises the nucleotide sequence of SEQ ID NO: 5, or a nucleotide sequence having at least 99%, 98%, 97%, 96%, 95%, 90%, 85%, or 80% identity to the nucleotide sequence of SEQ ID NO: 5; and/or
(iii) the second polypeptide chain comprises the amino acid sequence of SEQ ID NO:
6, or an amino acid sequence having at least 99%, 98%, 97%, 96%, 95%, 90%, 85%, or 80% identity to the amino acid sequence of SEQ ID NO: 6; and/or
(iv) the second RNA comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7; and/or
(v) the first RNA comprises the nucleotide sequence of SEQ ID NO: 5 and the second RNA comprises the nucleotide sequence of SEQ ID NO: 7.

42-45. (canceled)

46. The composition or medical preparation of claim 1, wherein
(i) the RNA is mRNA; and/or
(ii) the RNA is formulated as a liquid, formulated as a solid, or a combination thereof; and/or
(iii) the RNA is formulated or is to be formulated for injection; and/or
(iv) the RNA is formulated or is to be formulated for intravenous administration.

47-49. (canceled)

50. The composition or medical preparation of claim 1, wherein the RNA is formulated or is to be formulated as particles, wherein the particles optionally are lipid nanoparticles (LNP).

51. (canceled)

52. (canceled)

53. The composition or medical preparation of claim 1, which is a pharmaceutical composition, wherein the pharmaceutical composition preferably comprises a dose of 0.05 μg/kg or more, or 0.05 μg/kg to 5 mg/kg, or 0.05 μg/kg to 500 μg/kg, or 0.5 μg/kg to 500 μg/kg, or 1 μg/kg to 50 μg/kg, or 5 μg/kg to 150 μg/kg, or 15 μg/kg to 150 μg/kg RNA encoding the first and second polypeptide, wherein kg refers to kg body weight of a subject to be treated, wherein the pharmaceutical composition optionally further comprises one or more pharmaceutically acceptable carriers, diluents and/or excipients.

54. (canceled)

55. The composition or medical preparation of claim 1, wherein the medical preparation is a kit, wherein optionally
(i) the RNA and optionally the particle forming components are in separate vials; and/or
(ii) the kit further comprises instructions for use of the composition or medical preparation for treating or preventing cancer.

56-65. (canceled)

66. A method of treating cancer in a subject, the method comprising administering to the subject the composition or medical preparation of claim 1.

67-112. (canceled)

113. The method of claim 66, wherein
(i) the RNA is administered by injection, preferably once weekly; and/or
(ii) the RNA is administered by intravenous administration.

114-119. (canceled)

120. The method of claim 66, which further comprises administering a further therapy, optionally selected from the group consisting of: (i) surgery to excise, resect, or debulk a tumor, (ii) radiotherapy, (iii) chemotherapy and (iv) administering a further therapeutic agent.

121. (canceled)

122. (canceled)

123. The method of claim 120, wherein the further therapeutic agent comprises an anti-cancer therapeutic agent.

124. The method of claim 66, wherein the subject is a human.

125. The method of claim 66, wherein the cancer is CLDN6-positive cancer.

126. (canceled)

127. A method for therapeutic or prophylactic treatment of a disease or disorder in a subject, the method comprising administering to the subject the composition or medical preparation of claim 1.


SEQUENCE LISTING (36 sequences)

SEQ ID NO: 1 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 2 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAVIRDFYNPLVAEAQKRELGASLYLGWAASGLLLLGGGLLCCTCPSGGSQGPSHYMARYSTSAPAISRGPSEYPTKNYV

SEQ ID NO: 3 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 4 - protein, 510 aa, synthetic construct
  REGION 1..510: note = Fd sequence
MRVMAPRTLILLLSGALALTETWAGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSGPGGGRSGGGGSGGGGSEVQLQQSGPELVKPGASMKISCKASGYSFTGYTMNWVKQSHGKCLEWIGLINPYNGGTIYNQKFKGKATLTVDKSSSTAYMELLSLTSEDSAVYYCARDYGFVLDYWGQGTTLTVSSGGGGSGGGGSGGGGSGGGGSQIVLTQSPSIMSVSPGEKVTITCSASSSVSYMHWFQQKPGTSPKLSIYSTSNLASGVPARFSGRGSGTSYSLTISRVAAEDAATYYCQQRSNYPPWTFGCGTKLEIK

SEQ ID NO: 5 - RNA, 1997 nt, synthetic construct
  misc_feature 1..1997: note = Fd sequence
  source 1..1997: mol_type = other RNA
agaataaactagtattcttctggtccccacagactcagagagaacccgccaccatgagagtgatggcccctagaacactgatcctgctgctgtctggtgccctggctctgacagaaacatgggccggatctcaggtgcagctccagcaatctggtgccgaacttgctagacctggcgcctccgtgaagatgagctgtaaaaccagcggctacaccttcacacggtacaccatgcactgggtcaagcagaggcctggacagggccttgagtggatcggctacatcaaccccagccggggctacaccaactacaaccagaagttcaaggacaaggccacactgaccaccgacaagagcagcagcacagcctacatgcagctgagcagcctgaccagcgaagatagcgccgtgtactactgcgcccggtactacgacgatcactacagcctggattactggggccagggaacaaccctgacagtgtctagcgccagcaccaagggacctagcgttttcccactggctcccagcagcaagagcacatctggtggaacagccgctctgggctgcctggtcaaggattactttcccgagcctgtgaccgtgtcctggaattctggcgctctgacaagcggcgtgcacacctttccagccgtgctgcaaagcagcggcctgtactctctgagcagcgtggtcacagtgcctagctctagcctgggcacccagacctacatctgcaatgtgaaccacaagcctagcaacaccaaggtggacaagagagtggaacccaagagctgttctggacccggcggaggaagatctggcggaggcggttctggtggcggaggatctgaagttcagctgcaacagtctggccccgagctggttaagcctggggcctctatgaagatctcctgcaaggcctccggctacagctttaccggctacacaatgaattgggttaagcagtcccacggcaagtgcctggaatggatcggcctgatcaacccttacaacggcggcaccatctataatcagaagtttaaaggcaaggctaccctcaccgtggacaagtctagctccaccgcctacatggaactgctgagcctgacctctgaggactccgccgtgtattattgtgccagagactacggcttcgtgctggactattggggacagggcactacactgactgtgtccagtggcggtggtggcagtggcggcggaggtagcggaggtggtggaagcggaggcggaggctctcaaattgtgctgacacagagccccagcatcatgagcgttagccctggcgagaaagtgaccatcacatgcagcgccagctcctccgtgtcctatatgcactggtttcagcagaagcccggcacaagccccaagctgtcgatctacagcaccagcaacctggccagcggagtgcctgccagattttctggtagaggcagcggcaccagctactccctgacaatctctagagtggccgccgaagatgccgccacctactactgtcagcagcggagcaattaccctccttggacctttggctgcggcaccaagctggaaatcaagtgatgaggatccgatctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 6 - protein, 489 aa, synthetic construct
  REGION 1..489: note = L sequence
MRVMAPRTLILLLSGALALTETWAGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSEVQLQQSGPELVKPGASMKISCKASGYSFTGYTMNWVKQSHGKCLEWIGLINPYNGGTIYNQKFKGKATLTVDKSSSTAYMELLSLTSEDSAVYYCARDYGFVLDYWGQGTTLTVSSGGGGSGGGGSGGGGSGGGGSQIVLTQSPSIMSVSPGEKVTITCSASSSVSYMHWFQQKPGTSPKLSIYSTSNLASGVPARFSGRGSGTSYSLTISRVAAEDAATYYCQQRSNYPPWTFGCGTKLEIK

SEQ ID NO: 7 - RNA, 1934 nt, synthetic construct
  misc_feature 1..1934: note = L sequence
  source 1..1934: mol_type = other RNA
agaataaactagtattcttctggtccccacagactcagagagaacccgccaccatgagagtgatggcccctagaacactgatcctgctgctgtctggtgccctggctctgacagaaacatgggccggatctcagatcgtgctgacacagagccctgccatcatgagtgcctctccaggcgagaaagtgaccatgacctgtagagccagcagcagcgtgtcctacatgaactggtatcagcagaagtccggcacaagccccaagcggtggatctacgatacaagcaaggtggccagcggcgtgccctacagattttctggctctggcagcggcaccagctacagcctgacaatcagcagcatggaagccgaggatgccgccacctactactgccagcagtggtccagcaatcccctgacatttggagccggcaccaagctggaactgaagcggacagttgccgctcctagcgtgttcatcttcccaccttccgacgagcagctgaagtctggaacagccagcgtcgtgtgcctgctgaacaacttctaccctcgggaagccaaggtgcagtggaaggtggacaatgccctccagtccggcaacagccaagagagcgtgaccgagcaggacagcaaggactccacctatagcctgagcagcaccctgacactgagcaaggccgactacgagaaacacaaggtgtacgcctgcgaagtgacccaccagggactgtctagccctgtgaccaagagcttcaacagaggcgagtgtgatgtgcctggcggctctgaagttcagctccagcagtctggacccgagctggttaagcctggcgcctccatgaagatctcttgcaaggcctccggctacagcttcaccggctacaccatgaattgggtcaagcagagccacggcaagtgcctggaatggatcggcctgatcaacccctacaacggcggcacaatctacaaccagaagttcaagggcaaagccacactgaccgtggacaagagcagcagcaccgcctatatggaactgctgagcctgaccagcgaggactccgccgtgtactactgcgccagagattacggcttcgtgctggactattggggccagggaacaaccctgacagtgtctagcggaggcggaggatctggtggcggaggaagtggcggaggcggttctggcggtggtggatctcaaattgtcctgactcagtcccctagcatcatgagcgtgtcacccggggagaaagtgacaatcacctgttccgccagctcctccgtgtcctacatgcactggttccagcagaagcccggcacctcccccaagctgtccatctactccacctccaacctggcctccggcgtgcccgccagattctctggcagaggctccggcaccagctactccctgaccatctctagagtggccgccgaggacgctgccacatattattgtcagcagcggagcaactaccctccttggacctttggctgcggaacaaagctggaaatcaagtgatgaggatccgatctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 8 - RNA, 47 nt, synthetic construct
  misc_feature 1..47: note = 5'-UTR
  source 1..47: mol_type = other RNA
aactagtattcttctggtccccacagactcagagagaacccgccacc

SEQ ID NO: 9 - RNA, 278 nt, synthetic construct
  misc_feature 1..278: note = 3'-UTR
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 10 - RNA, 110 nt, synthetic construct
  misc_feature 1..110: note = A30L70
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 11 - protein, 10 aa, synthetic construct
  REGION 1..10: note = Linker
GGGGSGGGGS

SEQ ID NO: 12 - protein, 15 aa, synthetic construct
  REGION 1..15: note = Linker
GGGGSGGGGSGGGGS

SEQ ID NO: 13 - protein, 20 aa, synthetic construct
  REGION 1..20: note = Linker
GGGGSGGGGSGGGGSGGGGS

SEQ ID NO: 14 - protein, 25 aa, synthetic construct
  REGION 1..25: note = Linker
GGGGSGGGGSGGGGSGGGGSGGGGS

SEQ ID NO: 15 - protein, 30 aa, synthetic construct
  REGION 1..30: note = Linker
GGGGSGGGGSGGGGSGGGGSGGGGSGGGGS

SEQ ID NO: 16 - protein, 18 aa, synthetic construct
  REGION 1..18: note = Linker
SGPGGGRSGGGGSGGGGS

SEQ ID NO: 17 - protein, 6 aa, synthetic construct
  REGION 1..6: note = Linker
DVPGGS

SEQ ID NO: 18 - protein, 8 aa, synthetic construct
  REGION 1..8: note = CDR
GYTFTRYT

SEQ ID NO: 19 - protein, 8 aa, synthetic construct
  REGION 1..8: note = CDR
INPSRGYT

SEQ ID NO: 20 - protein, 12 aa, synthetic construct
  REGION 1..12: note = CDR
ARYYDDHYSLDY

SEQ ID NO: 21 - protein, 12 aa, synthetic construct
  REGION 1..12: note = CDR
ARYYDDHYCLDY

SEQ ID NO: 22 - protein, 5 aa, synthetic construct
  REGION 1..5: note = CDR
SSVSY

SEQ ID NO: 23 - skipped

SEQ ID NO: 24 - protein, 9 aa, synthetic construct
  REGION 1..9: note = CDR
QQWSSNPLT

SEQ ID NO: 25 - protein, 8 aa, synthetic construct
  REGION 1..8: note = CDR
GYSFTGYT

SEQ ID NO: 26 - protein, 8 aa, synthetic construct
  REGION 1..8: note = CDR
INPYNGGT

SEQ ID NO: 27 - protein, 10 aa, synthetic construct
  REGION 1..10: note = CDR
ARDYGFVLDY

SEQ ID NO: 28 - protein, 5 aa, synthetic construct
  REGION 1..5: note = CDR
SSVSY

SEQ ID NO: 29 - skipped

SEQ ID NO: 30 - protein, 10 aa, synthetic construct
  REGION 1..10: note = CDR
QQRSNYPPWT

SEQ ID NO: 31 - protein, 510 aa, synthetic construct
  REGION 1..510: note = Fd sequence
MRVMAPRTLILLLSGALALTETWAGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSGPGGGRSGGGGSGGGGSEVQLQQSGPELVKPGASMKISCKASGYSFTGYTMNWVKQSHGKCLEWIGLINPYNGGTIYNQKFKGKATLTVDKSSSTAYMELLSLTSEDSAVYYCARDYGFVLDYWGQGTTLTVSSGGGGSGGGGSGGGGSGGGGSQIVLTQSPSIMSVSPGEKVTITCSASSSVSYMHWFQQKPGTSPKLWIYSTSNLASGVPARFSGRGSGTSYSLTISRVAAEDAATYYCQQRSNYPPWTFGCGTKLEIK

SEQ ID NO: 32 - RNA, 1997 nt, synthetic construct
  misc_feature 1..1997: note = Fd sequence
  source 1..1997: mol_type = other RNA
agaataaactagtattcttctggtccccacagactcagagagaacccgccaccatgagagtgatggcccctagaacactgatcctgctgctgtctggtgccctggctctgacagaaacatgggccggatctcaggtgcagctccagcaatctggtgccgaacttgctagacctggcgcctccgtgaagatgagctgtaaaaccagcggctacaccttcacacggtacaccatgcactgggtcaagcagaggcctggacagggccttgagtggatcggctacatcaaccccagccggggctacaccaactacaaccagaagttcaaggacaaggccacactgaccaccgacaagagcagcagcacagcctacatgcagctgagcagcctgaccagcgaagatagcgccgtgtactactgcgcccggtactacgacgatcactacagcctggattactggggccagggaacaaccctgacagtgtctagcgccagcaccaagggacctagcgttttcccactggctcccagcagcaagagcacatctggtggaacagccgctctgggctgcctggtcaaggattactttcccgagcctgtgaccgtgtcctggaattctggcgctctgacaagcggcgtgcacacctttccagccgtgctgcaaagcagcggcctgtactctctgagcagcgtggtcacagtgcctagctctagcctgggcacccagacctacatctgcaatgtgaaccacaagcctagcaacaccaaggtggacaagagagtggaacccaagagctgttctggacccggcggaggaagatctggcggaggcggttctggtggcggaggatctgaagttcagctgcaacagtctggccccgagctggttaagcctggggcctctatgaagatctcctgcaaggcctccggctacagctttaccggctacacaatgaattgggttaagcagtcccacggcaagtgcctggaatggatcggcctgatcaacccttacaacggcggcaccatctataatcagaagtttaaaggcaaggctaccctcaccgtggacaagtctagctccaccgcctacatggaactgctgagcctgacctctgaggactccgccgtgtattattgtgccagagactacggcttcgtgctggactattggggacagggcactacactgactgtgtccagtggcggtggtggcagtggcggcggaggtagcggaggtggtggaagcggaggcggaggctctcaaattgtgctgacacagagccccagcatcatgagcgttagccctggcgagaaagtgaccatcacatgcagcgccagctcctccgtgtcctatatgcactggtttcagcagaagcccggcacaagccccaagctgtggatctacagcaccagcaacctggccagcggagtgcctgccagattttctggtagaggcagcggcaccagctactccctgacaatctctagagtggccgccgaagatgccgccacctactactgtcagcagcggagcaattaccctccttggacctttggctgcggcaccaagctggaaatcaagtgatgaggatccgatctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 33 - protein, 489 aa, synthetic construct
  REGION 1..489: note = L sequence
MRVMAPRTLILLLSGALALTETWAGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSEVQLQQSGPELVKPGASMKISCKASGYSFTGYTMNWVKQSHGKCLEWIGLINPYNGGTIYNQKFKGKATLTVDKSSSTAYMELLSLTSEDSAVYYCARDYGFVLDYWGQGTTLTVSSGGGGSGGGGSGGGGSGGGGSQIVLTQSPSIMSVSPGEKVTITCSASSSVSYMHWFQQKPGTSPKLWIYSTSNLASGVPARFSGRGSGTSYSLTISRVAAEDAATYYCQQRSNYPPWTFGCGTKLEIK

SEQ ID NO: 34 - RNA, 1934 nt, synthetic construct
  misc_feature 1..1934: note = L sequence
  source 1..1934: mol_type = other RNA
agaataaactagtattcttctggtccccacagactcagagagaacccgccaccatgagagtgatggcccctagaacactgatcctgctgctgtctggtgccctggctctgacagaaacatgggccggatctcagatcgtgctgacacagagccctgccatcatgagtgcctctccaggcgagaaagtgaccatgacctgtagagccagcagcagcgtgtcctacatgaactggtatcagcagaagtccggcacaagccccaagcggtggatctacgatacaagcaaggtggccagcggcgtgccctacagattttctggctctggcagcggcaccagctacagcctgacaatcagcagcatggaagccgaggatgccgccacctactactgccagcagtggtccagcaatcccctgacatttggagccggcaccaagctggaactgaagcggacagttgccgctcctagcgtgttcatcttcccaccttccgacgagcagctgaagtctggaacagccagcgtcgtgtgcctgctgaacaacttctaccctcgggaagccaaggtgcagtggaaggtggacaatgccctccagtccggcaacagccaagagagcgtgaccgagcaggacagcaaggactccacctatagcctgagcagcaccctgacactgagcaaggccgactacgagaaacacaaggtgtacgcctgcgaagtgacccaccagggactgtctagccctgtgaccaagagcttcaacagaggcgagtgtgatgtgcctggcggctctgaagttcagctccagcagtctggacccgagctggttaagcctggcgcctccatgaagatctcttgcaaggcctccggctacagcttcaccggctacaccatgaattgggtcaagcagagccacggcaagtgcctggaatggatcggcctgatcaacccctacaacggcggcacaatctacaaccagaagttcaagggcaaagccacactgaccgtggacaagagcagcagcaccgcctatatggaactgctgagcctgaccagcgaggactccgccgtgtactactgcgccagagattacggcttcgtgctggactattggggccagggaacaaccctgacagtgtctagcggaggcggaggatctggtggcggaggaagtggcggaggcggttctggcggtggtggatctcaaattgtcctgactcagtcccctagcatcatgagcgtgtcacccggggaaaaagtgacaatcacatgcagcgccagctcctccgtgtcttatatgcactggttccagcaaaagccagggacctctcctaagctctggatctacagcaccagcaacctggcctctggcgtgccagctagattttccggtagaggctccggcacctcttactccctgaccatctctagagtggccgccgaggacgctgccacatattattgtcagcagcggagcaactaccctccttggacctttggctgcggaacaaagctggaaatcaagtgatgaggatccgatctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 35 - RNA, 1536 nt, synthetic construct
  misc_feature 1..1536: note = Coding sequence
  source 1..1536: mol_type = other RNA
atgagagtgatggcccctagaacactgatcctgctgctgtctggtgccctggctctgacagaaacatgggccggatctcaggtgcagctccagcaatctggtgccgaacttgctagacctggcgcctccgtgaagatgagctgtaaaaccagcggctacaccttcacacggtacaccatgcactgggtcaagcagaggcctggacagggccttgagtggatcggctacatcaaccccagccggggctacaccaactacaaccagaagttcaaggacaaggccacactgaccaccgacaagagcagcagcacagcctacatgcagctgagcagcctgaccagcgaagatagcgccgtgtactactgcgcccggtactacgacgatcactacagcctggattactggggccagggaacaaccctgacagtgtctagcgccagcaccaagggacctagcgttttcccactggctcccagcagcaagagcacatctggtggaacagccgctctgggctgcctggtcaaggattactttcccgagcctgtgaccgtgtcctggaattctggcgctctgacaagcggcgtgcacacctttccagccgtgctgcaaagcagcggcctgtactctctgagcagcgtggtcacagtgcctagctctagcctgggcacccagacctacatctgcaatgtgaaccacaagcctagcaacaccaaggtggacaagagagtggaacccaagagctgttctggacccggcggaggaagatctggcggaggcggttctggtggcggaggatctgaagttcagctgcaacagtctggccccgagctggttaagcctggggcctctatgaagatctcctgcaaggcctccggctacagctttaccggctacacaatgaattgggttaagcagtcccacggcaagtgcctggaatggatcggcctgatcaacccttacaacggcggcaccatctataatcagaagtttaaaggcaaggctaccctcaccgtggacaagtctagctccaccgcctacatggaactgctgagcctgacctctgaggactccgccgtgtattattgtgccagagactacggcttcgtgctggactattggggacagggcactacactgactgtgtccagtggcggtggtggcagtggcggcggaggtagcggaggtggtggaagcggaggcggaggctctcaaattgtgctgacacagagccccagcatcatgagcgttagccctggcgagaaagtgaccatcacatgcagcgccagctcctccgtgtcctatatgcactggtttcagcagaagcccggcacaagccccaagctgtcgatctacagcaccagcaacctggccagcggagtgcctgccagattttctggtagaggcagcggcaccagctactccctgacaatctctagagtggccgccgaagatgccgccacctactactgtcagcagcggagcaattaccctccttggacctttggctgcggcaccaagctggaaatcaagtgatga

SEQ ID NO: 36 - RNA, 1473 nt, synthetic construct
  misc_feature 1..1473: note = Coding sequence
  source 1..1473: mol_type = other RNA
atgagagtgatggcccctagaacactgatcctgctgctgtctggtgccctggctctgacagaaacatgggccggatctcagatcgtgctgacacagagccctgccatcatgagtgcctctccaggcgagaaagtgaccatgacctgtagagccagcagcagcgtgtcctacatgaactggtatcagcagaagtccggcacaagccccaagcggtggatctacgatacaagcaaggtggccagcggcgtgccctacagattttctggctctggcagcggcaccagctacagcctgacaatcagcagcatggaagccgaggatgccgccacctactactgccagcagtggtccagcaatcccctgacatttggagccggcaccaagctggaactgaagcggacagttgccgctcctagcgtgttcatcttcccaccttccgacgagcagctgaagtctggaacagccagcgtcgtgtgcctgctgaacaacttctaccctcgggaagccaaggtgcagtggaaggtggacaatgccctccagtccggcaacagccaagagagcgtgaccgagcaggacagcaaggactccacctatagcctgagcagcaccctgacactgagcaaggccgactacgagaaacacaaggtgtacgcctgcgaagtgacccaccagggactgtctagccctgtgaccaagagcttcaacagaggcgagtgtgatgtgcctggcggctctgaagttcagctccagcagtctggacccgagctggttaagcctggcgcctccatgaagatctcttgcaaggcctccggctacagcttcaccggctacaccatgaattgggtcaagcagagccacggcaagtgcctggaatggatcggcctgatcaacccctacaacggcggcacaatctacaaccagaagttcaagggcaaagccacactgaccgtggacaagagcagcagcaccgcctatatggaactgctgagcctgaccagcgaggactccgccgtgtactactgcgccagagattacggcttcgtgctggactattggggccagggaacaaccctgacagtgtctagcggaggcggaggatctggtggcggaggaagtggcggaggcggttctggcggtggtggatctcaaattgtcctgactcagtcccctagcatcatgagcgtgtcacccggggagaaagtgacaatcacctgttccgccagctcctccgtgtcctacatgcactggttccagcagaagcccggcacctcccccaagctgtccatctactccacctccaacctggcctccggcgtgcccgccagattctctggcagaggctccggcaccagctactccctgaccatctctagagtggccgccgaggacgctgccacatattattgtcagcagcggagcaactaccctccttggacctttggctgcggaacaaagctggaaatcaagtgatga